(19) United States
(12) Patent Application Publication
(10) Pub. No.: US 2011/0207745 A1
(43) Pub. Date: Aug. 25, 2011

(54) SOLID FORMS OF 4--N,N-DIETHYLBENZAMIDE, COMPOSITIONS THEREOF, AND USES THEREWITH

(75) Inventors: Louis Diorazio, Macclesfield (GB); Andrew Hornby Dobson, Macclesfield (GB); Alison Norton, Macclesfield (GB); Anne O'Kearney-McMullan, Macclesfield (GB); Dedong Wu, Wilmington, DE (US)

(73) Assignee: ASTRAZENECA AB, Sodertalje (SE)

(21) Appl. No.: 12/952,286

(22) Filed: Nov. 23, 2010

Related U.S. Application Data
(60) Provisional application No. 61/264,647, filed on Nov. 25, 2009.

(51) Int. Cl. A61K 31/4965; C07D 241/04; A61P 25/22; A61P 25/24; A61P 25/00

(52) U.S. Cl. 514/255.04; 544/400

(57) ABSTRACT

Solid forms comprising salts of 4-{(R)-(3-aminophenyl)[4-(4-fluorobenzyl)piperazin-1-yl]methyl}-N,N-diethylbenzamide, compositions comprising the solid forms, methods of making the solid forms, and methods of their use for the treatment of various diseases and/or disorders are provided herein.

Provided herein are solid forms including salts of 4-{(R)-(3-aminophenyl)[4-(4-fluorobenzyl)piperazin-1-yl]methyl}-N,N-diethylbenzamide, compositions comprising the solid forms, methods of making the solid forms, and methods of their use for the treatment of various diseases and/or disorders.

The delta (“δ”) receptor has been identified as having a role in many bodily functions such as nociceptive, motor, and cardiovascular systems, as well as in emotional regulation. Ligands for the δ receptor may therefore find potential use as analgesics, anxiolytics, and/or as antidepressant agents. Ligands for the δ receptor have also been shown to possess immunomodulatory activities.

The mu (“μ”), delta (“δ”), and kappa (“κ”) receptors are well-established opioid receptors apparent in both the central and peripheral nervous systems of many species, including humans. Receptor localization studies have shown that δ-opioid receptors reside in areas of the brain implicated in mood regulation. The δ-opioid receptor was first identified as a possible target for treating depression and anxiety when heightened anxiety states and depressive-like behaviors were consistently observed in the δ-opioid receptor knockout mouse. A decrease in pain and anxiety have been observed in various animal models when one or more δ-opioid receptors was activated. Additionally, a number of investigators have found selective δ-opioid receptor agonists have antidepressant-like properties in models such as the forced swim test.

Efforts have been undertaken to develop δ-opioid receptor ligands that are therapeutically effective in treating depression, anxiety, and/or pain. More specifically, efforts have focused on developing selective δ-opioid receptor ligands. Selective δ-opioid receptor ligands advantageously cause less side effects than non-selective δ-opioid receptor ligands.

The chemical structure of 4-{(R)-(3-aminophenyl)[4-(4-fluorobenzyl)piperazin-1-yl]methyl}-N,N-diethylbenzamide, was disclosed as a δ-agonist compound in U.S. Patent Application Publication No. 2006/0030569 A1, which published on Feb. 9, 2006.

The identification and selection of a solid form of a pharmaceutical compound is complex, given that a change in solid form may substantially yet unpredictably affect a variety of physical and chemical properties potentially relevant for processing, formulation, bioavailability, physical stability and/or chemical stability, among other important pharmaceutical characteristics. In general, potential pharmaceutical solids include crystalline solids and amorphous solids. Amorphous solids may be characterized by a lack of long-range structural order; crystalline solids may be characterized by structural periodicity. See, e.g., Vippagunta et al., Adv. Drug. Deliv. Rev., (2001) 48:3-26; Yu, Adv. Drug. Deliv. Rev., (2001) 48:27-42.

Single-component solids consist essentially of the pharmaceutical compound in the absence of other compounds. Variety among single-component crystalline materials may potentially arise from the phenomenon of polymorphism, wherein multiple three-dimensional arrangements exist for a particular pharmaceutical compound. See, e.g., Byrn et al., Solid State Chemistry of Drugs, (1999) SSCI, West Lafayette. The importance of discovering polymorphs was underscored by the case of ritonavir, an HIV protease inhibitor that was formulated as soft gelatin capsules. About two years after the product was launched, the unanticipated precipitation of a new, less soluble polymorph in the formulation necessitated the withdrawal of the product from the market until a more consistent formulation could be developed. See Chemburkar et al., Org. Process Res. Dev., (2000) 4:413-417.

Additional diversity among the potential solid forms of a pharmaceutical compound may arise from the possibility of multiple-component solids. Crystalline solids comprising two or more ionic species are termed salts. See, e.g., Handbook of Pharmaceutical Salts: Properties, Selection and Use, P. H. Stahl and C. G. Wermuth, Eds., (2002), Wiley, Weinheim. Additional types of multiple-component solids that may potentially offer other property improvements for a pharmaceutical compound or salt thereof include, e.g., solvates (e.g., hydrates). See, e.g., Byrn et al., Solid State Chemistry of Drugs, supra. Multiple-component crystal forms may be potentially susceptible to polymorphism, wherein a given multiple-component composition may exist in more than one three-dimensional crystalline arrangement. The discovery of solid forms is of great importance in the development of a safe, effective, stable and marketable pharmaceutical compound.

Accordingly, a need exists for new δ-opioid receptor ligands, new salts of δ-opioid receptor ligands, and new solid forms of δ-opioid receptor ligands, which have advantageous physical, chemical and/or biological properties for treating, preventing, or managing diseases and disorders including, but not limited to, anxiety, depression, pain, and anxious major depressive disorder (AMDD).

Provided herein are salts and solid forms of 4-{(R)-(3-aminophenyl)[4-(4-fluorobenzyl)piperazin-1-yl]methyl}-N,N-diethylbenzamide (hereinafter “Compound A1”), the chemical structure and preparation of which were disclosed in U.S. Patent Application Publication No. 2006/0030569 A1. Compound A1 is useful as a pharmaceutical compound for the treatment, prevention, or management of diseases or disorders related to, e.g., the central nervous system.

In certain embodiments, solid forms provided herein are crystal forms, including, but not limited to, crystal forms of salts of Compound A1. In certain embodiments, the crystal forms are solvated (e.g., hydrated). In certain embodiments, the solid forms are amorphous forms, including, but not limited to, amorphous forms of salts of Compound A1. Without intending to be limited by any particular theory, particular properties (e.g., storage stability, compressibility, bulk density or dissolution properties) of certain solid forms described herein are believed to be beneficial for manufacturing, formulation, storage and/or bioavailability of Compound A1.

In particular embodiments, solid forms provided herein include solid forms comprising Compound A1, including, but not limited to, particular solid forms comprising salts of Compound A1, such as, e.g., salts with hydrochloric acid (hydrochloride salts of Compound A1), salts with fumaric acid (fumarate salts of Compound A1), salts with sulfuric acid (sulfate salts of Compound A1), salts with phosphoric acid (phosphate salts of Compound A1), and salts with hydrobromic acid (hydrobromide salts of Compound A1). In particular embodiments, HCl salts comprising Compound A1 include mono-HCl salts, di-HCl salts, and tri-HCl salts of Compound A1. In certain embodiments, solid forms provided herein include polymorphs or solvates (including hydrates) comprising salts of Compound A1. Certain embodiments herein provide methods of making, isolating, and/or characterizing the solid forms provided herein.

Certain solid forms provided herein are the active pharmaceutical ingredient in a pharmaceutical composition useful in treating pain, depression, anxiety, and AMDD in a warm-blooded animal in need of such treatment. Thus, embodiments herein encompass the use of the solid forms described herein in a final drug product. Certain embodiments provide solid forms useful in making final dosage forms with improved properties that are beneficial for such final dosage form to possess. Certain embodiments herein provide pharmaceutical compositions comprising a multiple-component crystal form and/or a multiple-component amorphous form comprising a salt of Compound A1 and at least one pharmaceutically acceptable diluent, excipient or carrier. Certain solid forms and the final drug products provided herein are useful, for example, in treating, preventing, or managing diseases and disorders discussed herein.

Certain embodiments provide methods of using the solid forms provided herein or pharmaceutical compositions comprising the solid forms to treat, prevent or manage diseases and disorders including, but not limited to, for example, diseases or disorders of the central nervous system. Other embodiments are directed to methods for using the solid forms provided herein or pharmaceutical compositions comprising the solid forms to treat, prevent or manage diseases or disorders including, but not limited to, for example, diseases or disorders in which modulating the δ-opioid receptor ligand is beneficial. Certain embodiments provide methods for treating, preventing or managing diseases or disorders including, but not limited to, for example, depression, anxiety, pain, and AMDD, wherein such method comprises administering to a warm-blooded animal, e.g., a human, in need of such treatment, prevention or management a therapeutically effective amount of a solid form provided herein. Such diseases or disorders are further described herein.

### BRIEF DESCRIPTION OF THE DRAWINGS

FIG. 1 is a representative ¹H nuclear magnetic resonance spectroscopy (NMR) spectrum of the mono-HCl salt of Compound A1.

FIG. 2 is a representative X-ray powder diffraction (XRPD) pattern of Form I of the mono-HCl salt of Compound A1.

FIG. 3 is a representative XRPD pattern of Form I of the mono-HCl salt of Compound A1.

FIG. 4 is a comparison of three representative XRPD patterns of Form I of the mono-HCl salt of Compound A1.

FIG. 5 is a comparison of a representative experimental XRPD pattern (top) and a representative simulated XRPD pattern (bottom) of Form I of the mono-HCl salt of Compound A1.

FIG. 6 is a representative differential scanning calorimetry (DSC) thermogram (top) and representative thermogravimetric analysis (TGA) thermogram (bottom) of Form I of the mono-HCl salt of Compound A1.

FIG. 7 is a comparison of two representative DSC thermograms of Form I of the mono-HCl salt of Compound A1.

FIG. 8 is a comparison of two representative TGA thermograms of Form I of the mono-HCl salt of Compound A1.

FIG. 9 is a diagram of an asymmetric unit of Form I of the mono-HCl salt of Compound A1.

FIG. 10 is a representative polarized light microscopy (PLM) photomicrograph (at 100× magnification) of Form I of the mono-HCl salt of Compound A1.

FIG. 11 is a representative dynamic vapor sorption (DVS) isotherm plot of Form I of the mono-HCl salt of Compound A1.

FIG. 12 is a comparison of three representative XRPD patterns of the amorphous form of the mono-HCl salt of Compound A1.

FIG. 13 is a representative XRPD pattern of the amorphous form of the mono-HCl salt of Compound A1.

FIG. 14 is a representative DSC thermogram (top) and a representative TGA thermogram (bottom) of the amorphous form of the mono-HCl salt of Compound A1.

FIG. 15 is a representative DSC thermogram (top) and a representative TGA thermogram (bottom) of the amorphous form of the mono-HCl salt of Compound A1.

FIG. 16 is a representative temperature-cycling DSC thermogram of the amorphous form of the mono-HCl salt of Compound A1.

FIG. 17 is a representative temperature-cycling DSC thermogram of the amorphous form of the mono-HCl salt of Compound A1.

FIG. 18 is a representative modulated-DSC thermogram of the amorphous form of the mono-HCl salt of Compound A1.

FIG. 19 is a representative modulated-DSC thermogram of the amorphous form of the mono-HCl salt of Compound A1.

FIG. 20 is a representative DVS isotherm plot of the amorphous form of the mono-HCl salt of Compound A1.

FIG. 21 is a representative XRPD pattern of the amorphous form of the tri-HCl salt of Compound A1.

FIG. 22 is a representative DSC thermogram (bottom left-top right) and a representative TGA thermogram (top left-bottom right) of the amorphous form of the tri-HCl salt of Compound A1.

FIG. 23 is a representative DVS isotherm plot of the amorphous form of the tri-HCl salt of Compound A1.

FIG. 24 is a representative XRPD pattern of the amorphous form of the sulfate salt of Compound A1.

FIG. 25 is a representative DSC thermogram of the amorphous form of the sulfate salt of Compound A1.

FIG. 26 is a representative TGA thermogram of the amorphous form of the sulfate salt of Compound A1.

FIG. 27 is a representative DVS isotherm plot of the amorphous form of the sulfate salt of Compound A1.

FIG. 28 is a representative XRPD pattern of Form I of the mesylate salt of Compound A1.

FIG. 29 is a representative DVS isotherm of Form I of the mesylate salt of Compound A1.

FIG. 30 is a representative XRPD pattern of the amorphous form of the phosphate salt of Compound A1.

FIG. 31 is a representative DSC thermogram of the amorphous form of the phosphate salt of Compound A1.

FIG. 32 is a representative TGA thermogram of the amorphous form of the phosphate salt of Compound A1.

FIG. 33 provides a representative DVS isotherm plot of the amorphous form of the phosphate salt of Compound A1.

FIG. 34 is a representative XRPD pattern of Form I of the HBr salt of Compound A1.

FIG. 35 is a representative DSC thermogram of Form I of the HBr salt of Compound A1.

FIG. 36 is a representative TGA thermogram of Form I of the HBr salt of Compound A1.

FIG. 37 is a representative DVS isotherm plot of Form I of the HBr salt of Compound A1.

FIG. 38 is a representative XRPD pattern of Form I of the sesquifumarate salt of Compound A1.

FIG. 39 is a representative DSC thermogram and representative TGA thermogram of Form I of the sesquifumarate salt of Compound A1.

FIG. 40 is a representative DVS isotherm plot of Form I of the sesquifumarate salt of Compound A1.

FIG. 41 is a representative infrared (IR) spectrum of Form I of the sesquifumarate salt of Compound A1.

The definitions set forth herein take precedence over definitions set forth in any patent, patent application, and/or patent application publication incorporated herein by reference.

Definitions of terms used in describing the invention are set forth hereinbelow. Unless otherwise specified, the initial definition provided for a term applies each time such term is used.

The term “pharmaceutically acceptable salts” refers to salts prepared from a pharmaceutically acceptable acid, as known in the art. Examples herein, suitable acids and methods for preparing and analyzing salts are provided, e.g., in Handbook of Pharmaceutical Salts: Properties, Selection and Use, P. H. Stahl and C. G. Wermuth, Eds., (2002), Wiley, Weinheim; Gould, Int. J. Pharm. (1986) 33:201-17; and Serajuddin, Adv. Drug Deliv. Rev. (2007) 59:603-16.

The term “solid form” and related terms refer to a physical form which is not predominantly in a liquid or a gaseous state.

The term “solid form” and related terms, when used herein to refer to Compound A1, refer to a physical form comprising Compound A1 which is not predominantly in a liquid or a gaseous state. Solid forms may be crystalline, amorphous or a mixture thereof A “single-component” solid form comprising Compound A1 consists essentially of Compound A1. A “multiple-component” solid form comprising Compound A1 comprises a significant quantity of one or more additional species, such as ions and/or solvent molecules, within the solid form. For example, in particular embodiments, a crystalline multiple-component solid form comprising Compound A1 further comprises one or more species non-covalently bonded at regular positions in the crystal lattice.

The term “crystalline” and related terms when used to describe a substance, modification, material, component or product mean the substance, modification, material, component or product is substantially crystalline as determined, e.g., by X-ray diffraction, polarized light microscopy (PLM), and/or moisture sorption analysis, as known in the art. See, e.g., Remington: The Science and Practice of Pharmacy, 21^{st} edition, Lippincott, Williams and Wilkins, Baltimore, Md. (2005); Byrn et al., Solid State Chemistry of Drugs, supra; The United States Pharmacopeia, (1995) 23rd ed.

The term “crystal forms” and related terms refer to solid forms that are crystalline. Crystal forms may include single-component crystal forms and multiple-component crystal forms, and include, but are not limited to, polymorphs and solvates (including hydrates), as well as salts, solvates of salts (including hydrates of salts), and polymorphs thereof. In certain embodiments, a crystal form is “substantially crystalline,” as determined, e.g., by XRPD, polarized light microscopy (PLM), and/or moisture sorption analysis. In specific embodiments, samples of “substantially crystalline” crystal forms are about 50%, about 55%, about 60%, about 65%, about 70%, about 75%, about 80%, about 85%, about 90%, about 91%, about 92%, about 93%, about 94%, about 95%, about 96%, about 97%, about 98%, about 99%, or about 100% crystalline. In certain embodiments, a crystal form of a substance may be substantially free of one or more amorphous forms and/or other crystal forms. In certain embodiments, a crystal form of a substance may be “physically pure,” i.e., contains less than about 1%, 2%, 3%, 4%, 5%, 6%, 7%, 8%, 9%, 10%, 15%, 20%, 25%, 30%, 35%, or 40% of other crystal forms or amorphous forms on a weight basis. In certain embodiments, a crystal form of a substance may be “chemically pure,” i.e. contains less than about 1%, 2%, 3%, 4%, 5%, 6%, 7%, 8%, 9%, 10%, 15%, 20%, 25%, 30%, 35%, or 40% of other chemical substances on a weight basis.

The terms “polymorphs,” “polymorphic forms” and related terms refer to two or more crystal forms that consist essentially of the same molecule, molecules and/or ions.

The terms “solvate” and “solvated” refer to a solid form of a substance which contains solvent. The terms “hydrate” and “hydrated” refer to a solvate wherein the solvent comprises water. “Polymorphs of solvates” refers to the existence of more than one crystal form for a particular solvate composition. Similarly, “polymorphs of hydrates” refers to the existence of more than one crystal form for a particular hydrate composition. The term “desolvated solvate” refers to a crystal form of a substance which may be prepared by removing the solvent from a solvate.

The term “amorphous,” “amorphous form,” and related terms mean the substance, component or product in question is not substantially crystalline as determined by X-ray diffraction. In particular, the term “amorphous form” describes a disordered solid form, i.e. a solid form lacking long range crystalline order. In certain embodiments, an amorphous form is “substantially amorphous,” as determined, e.g. by XRPD, PLM, and/or moisture sorption analysis. In specific embodiments, samples of “substantially amorphous” amorphous forms are about 60%, about 65%, about 70%, about 75%, about 80%, about 85%, about 90%, about 91%, about 92%, about 93%, about 94%, about 95%, about 96%, about 97%, about 98%, about 99%, or about 100% amorphous. In certain embodiments, an amorphous form of a substance may be substantially free of one or more other amorphous forms and/or crystal forms. In certain embodiments, an amorphous form of a substance may be “physically pure,” i.e. contains less than about 1%, 2%, 3%, 4%, 5%, 6%, 7%, 8%, 9%, 10%, 15%, 20%, 25%, 30%, 35%, or 40% of other amorphous forms or crystal forms on a weight basis. In certain embodiments, an amorphous form of a substance may be “chemically pure,” i.e. contains less than about 1%, 2%, 3%, 4%, 5%, 6%, 7%, 8%, 9%, 10%, 15%, 20%, 25%, 30%, 35%, or 40% of other chemical substances on a weight basis.

A sample or composition that is “substantially free” of one or more other solid forms and/or other chemical compounds means that the composition contains, in particular embodiments, less than about 25, 20, 15, 10, 9, 8, 7, 6, 5, 4, 3, 2, 1, 0.75, 0.5, 0.25 or 0.1 percent by weight of one or more other solid forms and/or other chemical compounds.

The terms “about” and “approximately” when used in connection with a numeric value or range of values used to characterize a particular solid form mean the value or range of values may deviate to an extent deemed reasonable to one of ordinary skill in the art while still describing the particular solid form. A numeric value or range of values that may be used to characterize a particular solid form include, for example, a specific temperature or temperature range that describes, for example, a melting, dehydration, desolvation or glass transition temperature; a mass change, such as, for example, a mass change as a function of temperature or humidity; a solvent or water content, in terms of, for example, mass or a percentage; or a peak position, such as, for example, in analysis by IR or Raman spectroscopy or XRPD

The term “match,” “matches,” “matching,” and related terms, when used to describe the relationship between particular analytical data items (such as, e.g., XRPD patterns, DSC thermograms, TGA thermograms, or DVS isotherm plots), mean that the particular analytical data items are equivalent, to an extent deemed reasonable to one of ordinary skill in the art. Factors that one of ordinary skill would consider in determining whether particular analytical data items match include, e.g., routine sample-to-sample variation, analytical errors, limits of detection, and background noise.

The terms “ambient temperature,” “room temperature,” and related terms refer, in specific embodiments, to a temperature between about 15° C. and about 30° C., a temperature between about 20° C. and about 25° C., or a temperature of about 17° C., about 18° C., about 19° C., about 20° C., about 21° C., about 22° C., about 23° C., about 24° C., or about 25° C.

The terms “treat,” “treating,” and “treatment” refer to the eradication or amelioration of a disease or disorder, or of one or more symptoms associated with the disease or disorder. In certain embodiments, the terms refer to minimizing the spread or worsening of the disease or disorder resulting from the administration of one or more prophylactic or therapeutic agents to a subject with such a disease or disorder. In some embodiments, the terms refer to the administration of a compound provided herein, with or without other additional active agent, after the onset of symptoms of the particular disease.

The terms “prevent,” “preventing,” and “prevention” refer to the prevention of the onset, recurrence or spread of a disease or disorder, or of one or more symptoms thereof. In certain embodiments, the terms refer to the treatment with or administration of a compound provided herein, with or without other additional active compound, prior to the onset of symptoms, particularly to patients at risk of disease or disorders provided herein. The terms encompass the inhibition or reduction of a symptom of the particular disease. Patients with familial history of a disease in particular are candidates for preventive regimens in certain embodiments. In addition, patients who have a history of recurring symptoms are also potential candidates for the prevention. In this regard, the term “prevention” may be interchangeably used with the term “prophylactic treatment.”

The terms “manage,” “managing,” and “management” refer to preventing or slowing the progression, spread after occurrence, or worsening of a disease or disorder, or of one or more symptoms thereof. Often, the beneficial effects that a subject derives from a prophylactic and/or therapeutic agent do not result in a cure of the disease or disorder. In this regard, the term “managing” encompasses treating a subject who had suffered from the particular disease in an attempt to prevent or minimize the recurrence of the disease.

The term “therapeutically-effective amount” refers to that amount of a compound sufficient to modulate one or more of the symptoms of the condition or disease being treated. A “therapeutically effective amount” and/or dosage range for compound used in the method of treatment of the invention may be determined by one of ordinary skill in the art via known criteria including age, weight, and response of the individual patient, and interpreted within the context of the disease being treated and/or prevented. Exemplary single or divided dosage amounts for a mammal may be from about 0.01 to about 300 mg/kg/day.

The phrase a “prophylactically effective amount” when used in connection with compound is an amount sufficient to prevent a disease or disorder, or prevent its recurrence. A prophylactically effective amount of a compound means an amount of therapeutic agent, alone or in combination with other agents, which provides a prophylactic benefit in the prevention of the disease. The term “prophylactically effective amount” can encompass an amount that improves overall prophylaxis or enhances the prophylactic efficacy of another prophylactic agent.

The term “composition” as used herein and unless otherwise specified is intended to encompass a product comprising the specified ingredients (and in the specified amounts, if indicated), as well as any product which results, directly or indirectly, from combination of the specified ingredients in the specified amounts. By “pharmaceutically acceptable” it is meant the diluent, excipient or carrier must be compatible with the other ingredients of the formulation and not deleterious to the recipient thereof.

The term “enantiomerically pure” refers to a compound containing at least 75% of the named enantiomer out of the total amount of the two possible enantiomers contained therein. In a particular embodiment, “enantiomerically pure” refers to a compound containing at least 90% of the named enantiomer out of the total amount of the two possible enantiomers contained therein. In a more particular embodiment, “enantiomerically pure” refers to a compound containing at least 95% of the named enantiomer out the total amount of the two possible enantiomers contained therein. In a yet further embodiment, “enantiomerically pure” refers to a compound containing at least 97% of the named enantiomer out the total amount of the two possible enantiomers contained therein. In still yet a further embodiment, “enantiomerically pure” refers to a compound containing at least 98% of the named enantiomer out the total amount of the two possible enantiomers contained therein. In a further embodiment, “enantiomerically pure” refers to a compound containing at least 99% of the named enantiomer out the total amount of the two possible enantiomers contained therein.

The term isolated means that a particular solid form, e.g., crystal form, has been substantially physically separated from the medium from which it was created.

To the extent there is a discrepancy between the chemical name of a compound and a depicted chemical structure of the compound provided herein, the chemical structure is preferred.

Solid forms provided herein may also comprise unnatural proportions of atomic isotopes at one or more of the atomic positions in Compound A1. For example, the compound may be substituted one or more positions with isotopes, such as, for example, deuterium (²H), tritium (³H), iodine-125 (¹²⁵I), sulfur-35 (³⁵S), or carbon-14 (¹⁴C). All isotopic variations of Compound A1, whether radioactive or not, are intended to be encompassed within the scope of the embodiments provided herein.

Embodiments that are, for clarity reasons, described herein in the context of separate embodiments, may also be combined to form a single embodiment. Conversely, various embodiments that are, for brevity reasons, described in the context of a single embodiment, may also be combined so as to form sub-combinations thereof. Unless specifically stated otherwise herein, references made in the singular may also include the plural. For example, “a” and “an” may refer to either one, or one or more. Embodiments identified herein as exemplary are intended to be illustrative and not limiting.

Certain embodiments herein provide multiple-component solid forms comprising Compound A1. Solid forms comprising salts of Compound A1 include crystal forms and amorphous forms, and include, but are not limited to, solvates (e.g., hydrates) and/or polymorphs. Particular embodiments herein provide amorphous forms comprising a pharmaceutically acceptable salt of Compound A1. Particular embodiments herein provide crystal forms comprising a pharmaceutically acceptable salt of Compound A1.

The term “Compound A1” means compound 4-{(R)-(3-aminophenyl)[4-(4-fluorobenzyl)piperazin-1-yl]methyl}-N,N-diethylbenzamide. In its free base form, Compound A1 has the following structure (I):

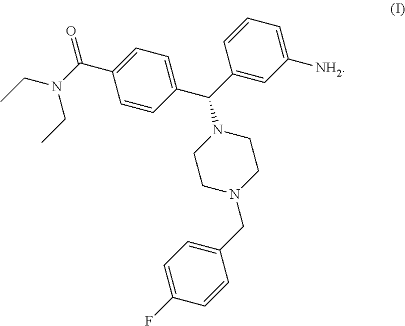

In specific embodiments, “Compound A1” includes ionized forms of the compound that have undergone salt formation such that the molecule is protonated at one or more atomic positions.

Compound A1 can be synthesized or obtained according to any method apparent to one of ordinary skill in the art, e.g. based upon the teachings herein. Compound A1 can also be prepared according to methods described in the following patent applications and publications, the entireties of each of which are incorporated by reference herein: Swedish Patent App. No. 0401968-3, filed Aug. 2, 2004; U.S. Provisional Patent App. No. 60/602,363, filed Aug. 18, 2004; and U.S. patent application Ser. No. 11/243,623, filed Oct. 5, 2005, published as U.S. Patent App. Publication No. 2006/0030569 A1 on Feb. 9, 2006.

Solid forms comprising Compound A1 can be prepared by the methods described herein, including the methods described in the Examples below, or by techniques including, but not limited to, heating, cooling, freeze drying, lyophilization, spray drying, quench cooling the melt, rapid solvent evaporation, slow solvent evaporation, solvent recrystallization, antisolvent addition, slurry recrystallization, crystallization from the melt, desolvation, recrystallization in confined spaces such as, e.g., in nanopores or capillaries, recrystallization on surfaces or templates such as, e.g., on polymers, recrystallization in the presence of additives, desolvation, dehydration, rapid cooling, slow cooling, exposure to solvent and/or water, drying, including, e.g., vacuum drying, vapor diffusion, sublimation, grinding (including, e.g., cryo-grinding and solvent-drop grinding), microwave-induced precipitation, sonication-induced precipitation, laser-induced precipitation and precipitation from a supercritical fluid. Unless otherwise specified, methods involving solvents described herein contemplate the use of any suitable common laboratory solvent, as known in the art (non-limiting examples of common laboratory solvents are provided, e.g., in Gottlieb et al., J. Org. Chem. (1997) 62:7515-15). The particle size of resulting solid forms, which can vary, (e.g., from nanometer dimensions to millimeter dimensions), can be controlled, e.g.: by varying crystallization conditions (such as, e.g., the rate of crystallization and/or the crystallization solvent system); by altering spray drying operating parameters (including, e.g., feed solution concentration); and/or equipment design or by particle-size reduction techniques (e.g., grinding, milling, micronizing or sonication).

While not intending to be bound by any particular theory, certain solid forms are characterized by properties, such as, for example, stability, solubility, dissolution rate, bioavailability and biological activity, appropriate for use as clinical and therapeutically active ingredients. Moreover, while not wishing to be bound by any particular theory, certain solid forms are characterized by properties, such as, for example, density, compressibility, hardness, morphology, powder flow, cleavage, stickiness, compaction, water uptake, electrical properties, thermal behavior, solubility, dissolution, solid-state reactivity, physical stability, chemical stability, and excipient compatibility that affect processes, such as, for example, yield, filtration, washing, drying, milling, mixing, tableting, formulation, storage, lyophilization, and other processing that make certain solid forms suitable for use in a solid dosage form. Such properties can be assessed using the particular analytical chemical techniques described herein or by methods known in the art.

In particular embodiments, certain solid forms described herein showed advantageous properties including properties relating to, e.g., water uptake, thermal behavior, solubility, dissolution, solid-state reactivity, physical stability, and/or chemical stability.

In particular embodiments, techniques suitable for characterizing certain solid forms provided herein include, but are not limited to, thermal gravimetric analysis (TGA), differential scanning calorimetry (DSC), X-ray powder diffractometry (XRPD), single-crystal X-ray diffractometry, vibrational spectroscopy, e.g., infrared (IR) and Raman spectroscopy, solid-state and solution nuclear magnetic resonance (NMR) spectroscopy, optical microscopy (e.g., polarized light microscopy (PLM)), hot stage optical microscopy, scanning electron microscopy (SEM), electron crystallography and quantitative analysis, particle size analysis (PSA), surface area analysis, solubility measurements, dissolution measurements, elemental analysis and Karl Fischer analysis. Characteristic unit cell parameters may be determined using one or more techniques such as, but not limited to, X-ray diffraction and neutron diffraction, including single-crystal diffraction and powder diffraction. Techniques useful for analyzing powder diffraction data include profile refinement, such as Rietveld refinement, which may be used, e.g., to analyze diffraction peaks associated with a single phase in a sample comprising more than one solid phase. Other methods useful for analyzing powder diffraction data include unit cell indexing, which allows one of skill in the art to determine unit cell parameters from a sample comprising crystalline powder.

In particular embodiments, solid forms provided herein have different physical properties such as, for example, melting temperatures, heats of fusion, solubilities, dissolution rates and/or vibrational spectra, as a result of, e.g., the arrangement or conformation of the molecules and/or ions in the solid forms. In certain embodiments, differences in physical properties may affect pharmaceutical parameters such as storage stability, compressibility and density (important in formulation and product manufacturing), and dissolution rate (an important factor in bioavailability). In certain embodiments, differences in stability result from changes in chemical reactivity (e.g., differential oxidation, such that a dosage form discolors more rapidly when comprised of one solid form than when comprised of another solid form) or mechanical changes (e.g., tablets crumble on storage as a kinetically favored solid form converts to a thermodynamically more stable solid form) or both (e.g., tablets of one solid form are more susceptible to breakdown at high humidity). In particular embodiments, such solubility and/or dissolution differences may affect potency and/or toxicity parameters. In certain embodiments, physical properties of solid forms provided herein may be important in processing (for example, one solid form might be more likely to form solvates or might be difficult to filter and wash free of impurities, and particle shape and size distribution might be different among solid forms).

Certain embodiments herein provide compositions comprising one or more of the solid forms provided herein. Certain embodiments provide compositions of one or more of the solid forms in combination with one or more other active ingredients. Certain embodiments provide methods of using these compositions in the treatment, prevention or management of diseases and disorders including, but not limited to, the diseases and disorders provided herein.

### Mono-HCl Salt of Compound A1

One embodiment is a monohydrochloride (“mono-HCl”) salt of Compound A1, which may be formed, e.g., by reacting Compound A1 with hydrochloric acid. In certain embodiments, a sample of the mono-HCl salt of Compound A1 comprises an amount of chloride ion per mole of Compound A1 equal to about 0.75, about 0.80, about 0.85, about 0.90, about 0.95, about 1.00, about 1.05, about 1.10, about 1.15, about 1.20, or about 1.25 molar equivalents of chloride ion per mole of Compound A1. In certain embodiments, the amount of hydrochloric acid per mole of Compound A1 is between about 0.75 and about 1.25, between about 0.80 and about 1.20, between about 0.85 and about 1.15, between about 0.90 and about 1.10, or between about 0.95 and about 1.05 molar equivalents of hydrochloric acid per mole of Compound A1.

As described herein, the mono-HCl salt of Compound A1 may be obtained, e.g., by reacting Compound A1 with hydrochloric acid under conditions suitable for obtaining the mono-HCl salt of Compound A1. For example, in certain embodiments, the mono-HCl may be formed by contacting a solution comprising the free base of Compound A1 with a solution comprising hydrochloric acid. In certain embodiments, the solution comprising Compound A1 can be formed from any suitable solvent system, such as a solvent system comprising, e.g., water, methanol, ethanol, isopropanol, ethyl acetate, isopropyl acetate, dichloromethane, petroleum ether, or mixture of two or more thereof. In certain embodiments, the solution comprising hydrochloric acid can be formed from any suitable solvent system, such as, e.g., a solvent system comprising water, methanol, ethanol, isopropanol, ethyl acetate, isopropyl acetate, dichloromethane, petroleum ether, or mixture of two or more thereof. In certain embodiments, the mono-HCl salt is obtained by contacting Compound A1 with approximately 1 molar equivalent of hydrochloric acid per mole of Compound A1. In a further embodiment, the reaction of Compound A1 with approximately 1 molar equivalent of hydrochloric acid per mole of Compound A1 is performed in a solvent.

In certain embodiments, a sample of the mono-HCl salt of Compound A1 is substantially free of one or more HCl salts of Compound A1 with a stoichiometry other than about 1:1. For example, in specific embodiments, a sample of the mono-HCl salt of Compound A1 is substantially free of a di-HCl salt of Compound A1. In specific embodiments, a sample of the mono-HCl salt of Compound A1 is substantially free of a tri-HCl salt of Compound A1. In specific embodiments, a sample of the mono-HCl salt of Compound A1 is substantially free of a di-HCl salt of Compound A1 and a tri-HCl salt of Compound A1.

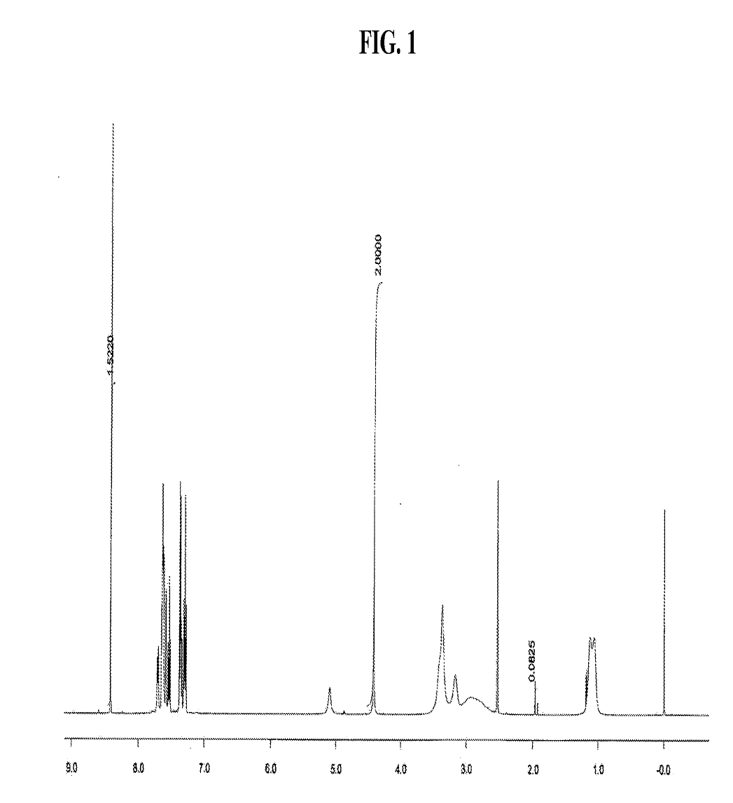

A representative solution ¹H NMR spectrum of the mono-HCl salt of Compound A1 is provided in FIG. 1.

### Form I of the Mono-HCl Salt of Compound A1

Certain embodiments herein provide Form I of the mono-HCl salt of Compound A1. In some embodiments, the Form I of the mono-HCl salt of Compound A1 is isolated.

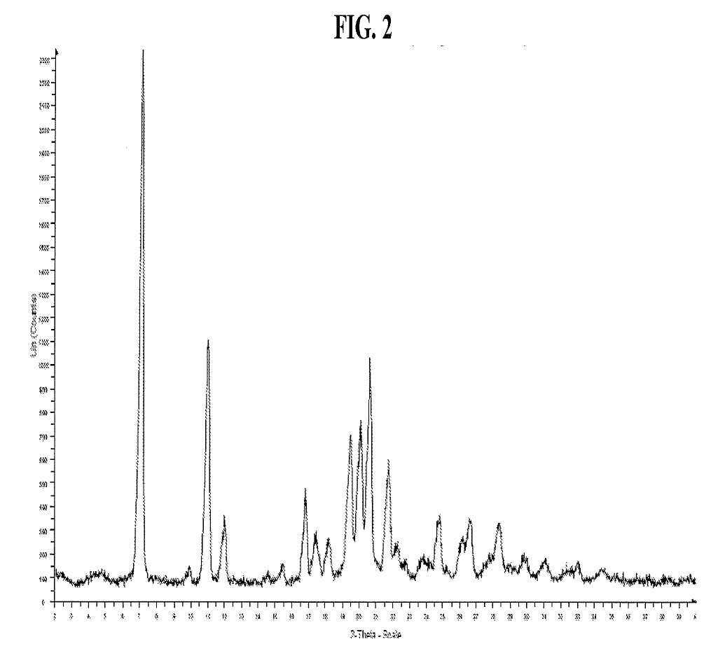

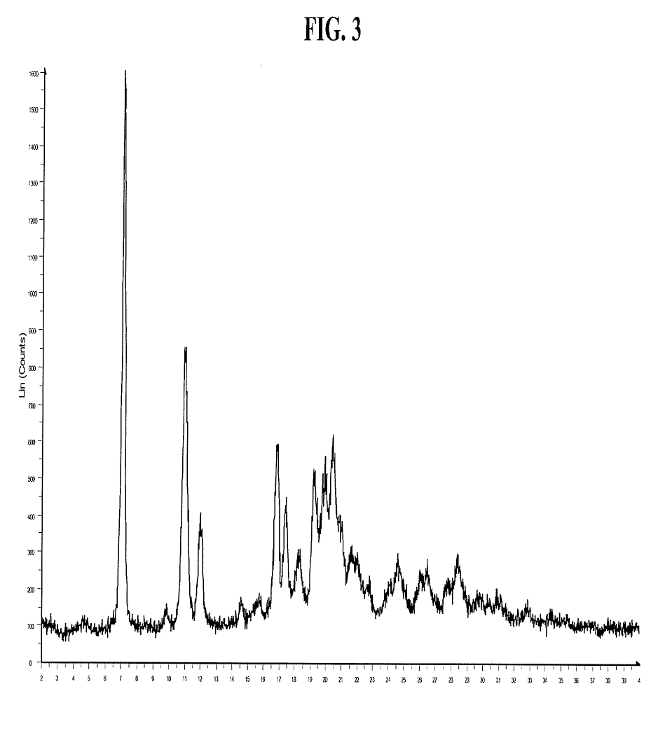

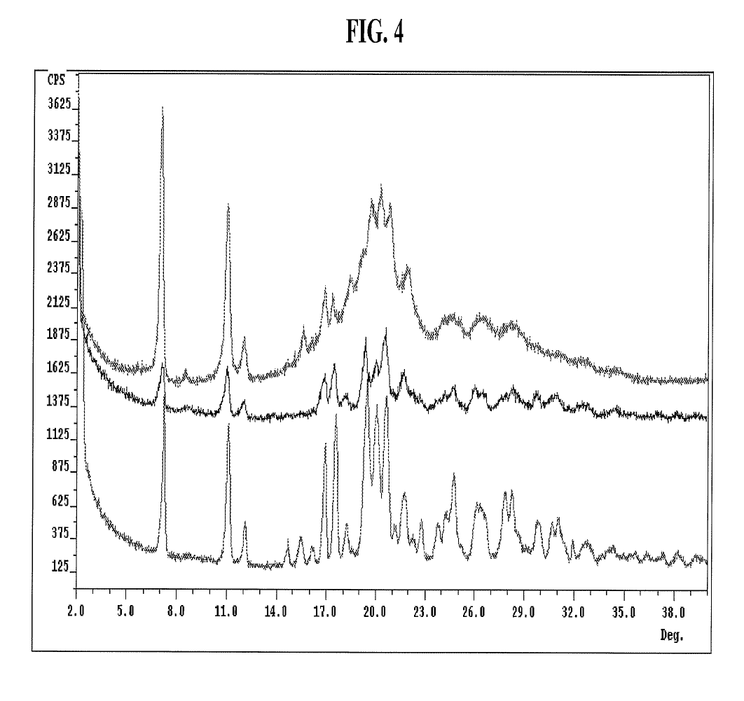

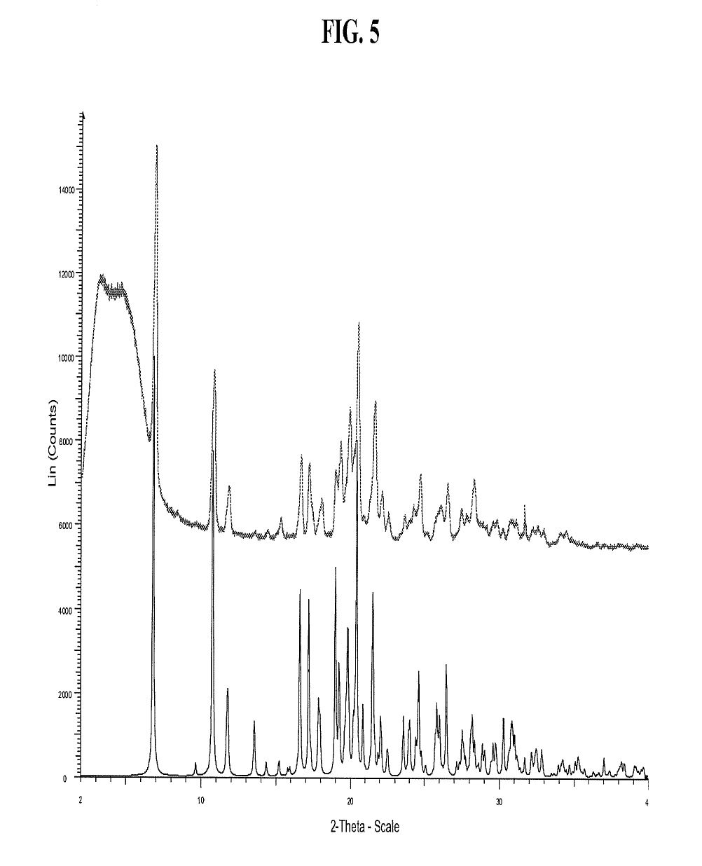

In certain embodiments, Form I of the mono-HCl salt of Compound A1 exhibits particular structural characteristics, as determined, e.g., by diffraction analysis. Representative XRPD patterns of Form I of the mono-HCl salt of Compound A1 are provided in FIG. 2, FIG. 3, and FIG. 4. In certain embodiments, Form I of the mono-HCl salt of Compound A1 is characterized by XRPD peaks located at any one, two, three, four, five, six, seven, eight, nine, ten, eleven, twelve, thirteen, fourteen or fifteen of the following approximate positions: 7.0, 11.0, 12.0, 14.5, 15.6, 16.8, 17.4, 18.2, 19.1, 19.3, 19.8, 20.3, 21.5, 24.6, and 26.5 degrees 2θ. In some embodiments, Form I of the mono-HCl salt of Compound A1 is characterized by at least 8, at least 9, or at least 10 of said approximate positions. In certain embodiments, Form I of the mono-HCl salt of Compound A1 is characterized by XRPD peaks at about 7.0, 11.0 and 16.8 degrees 2θ. In certain embodiments, Form I of the mono-HCl salt of Compound A1 is characterized by XRPD peaks at about 19.1, 19.8, and 20.3 degrees 2θ. In certain embodiments, Form I of the mono-HCl salt of Compound A1 is characterized by XRPD peaks at about 12.0, 17.4, and 19.3 degrees 2θ. In some embodiments, Form I of the mono-HCl salt of Compound A1 is characterized by XRPD peaks at about 7.0, 11.0, 12.0, 16.8, 17.4, 19.1, 19.3, 19.8, and 20.3 degrees 2θ. In some embodiments, Form I of the mono-HCl salt of Compound A1 is characterized by XRPD peaks at about 7.0, 11.0, 12.0, 16.8, 17.4, 18.2, 19.1, 19.3, 19.8, and 20.3 degrees 2θ. In certain embodiments, Form I of the mono-HCl salt of Compound A1 is characterized by an XRPD pattern which matches the pattern exhibited in FIG. 2. In certain embodiments, Form I of the mono-HCl salt of Compound A1 is characterized by an XRPD pattern which matches the pattern exhibited in FIG. 3 In certain embodiments, Form I of the mono-HCl salt of Compound A1 is characterized by an XRPD pattern which matches one, two or three of the XRPD patterns exhibited in FIG. 4. In certain embodiments, Form I of the mono-HCl salt of Compound A1 is characterized by an XRPD pattern which matches the experimental XRPD pattern or the simulated XRPD pattern exhibited in FIG. 5, or both. In particular embodiments, a sample of Form I of the mono-HCl salt of Compound A1 is substantially crystalline. In some embodiments, provided herein is an isolated Form I mono-HCl salt of Compound A1, which has an XRPD pattern which matches any of the patterns of FIG. 2, FIG. 3, FIG. 4 or FIG. 5. In some embodiments, provided herein is an isolated Form I mono-HCl salt of Compound A1, which has an XRPD pattern comprising peaks at about 7.0, 11.0 and 16.8 degrees 2θ.

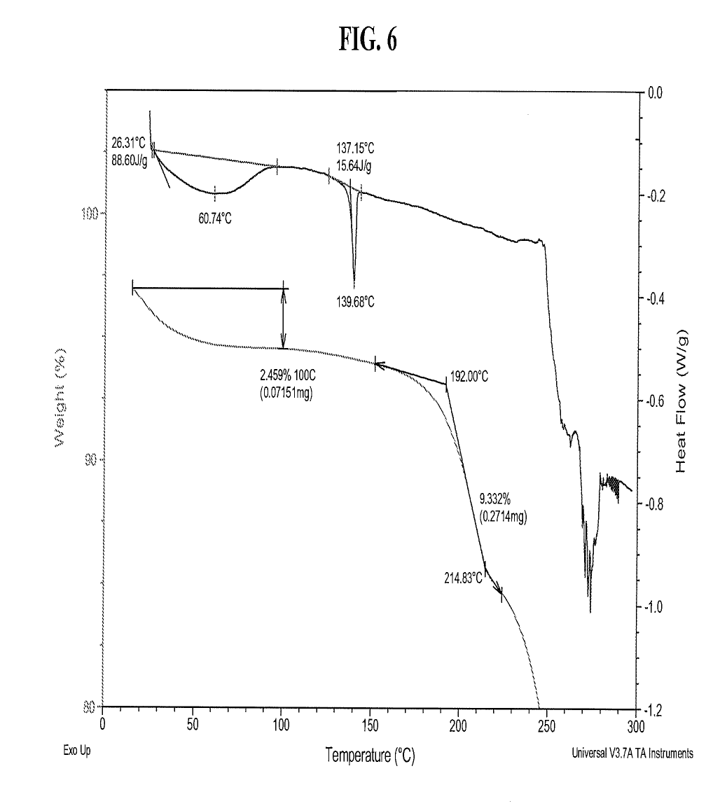

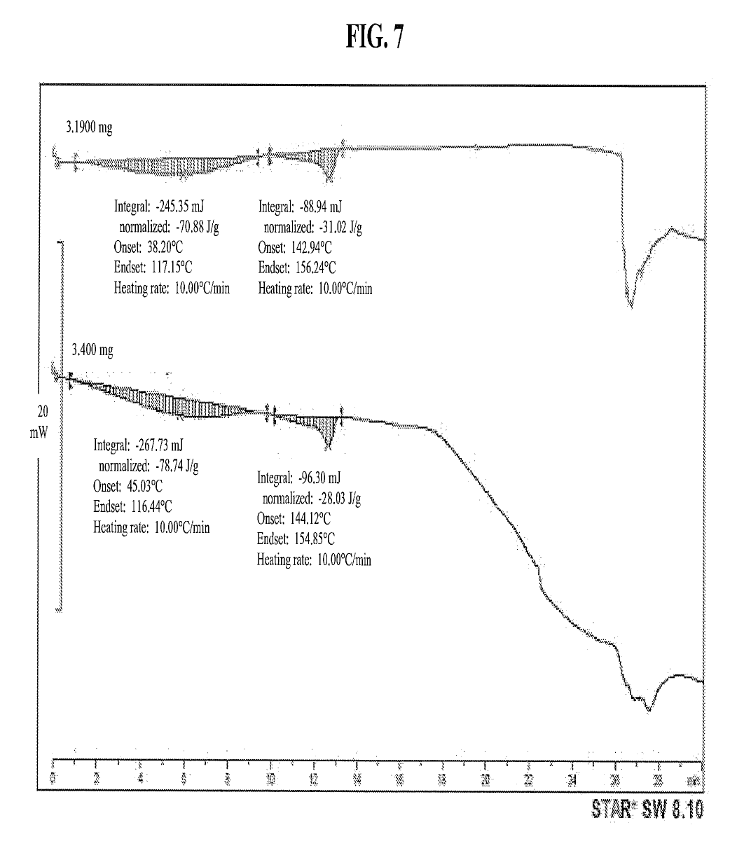

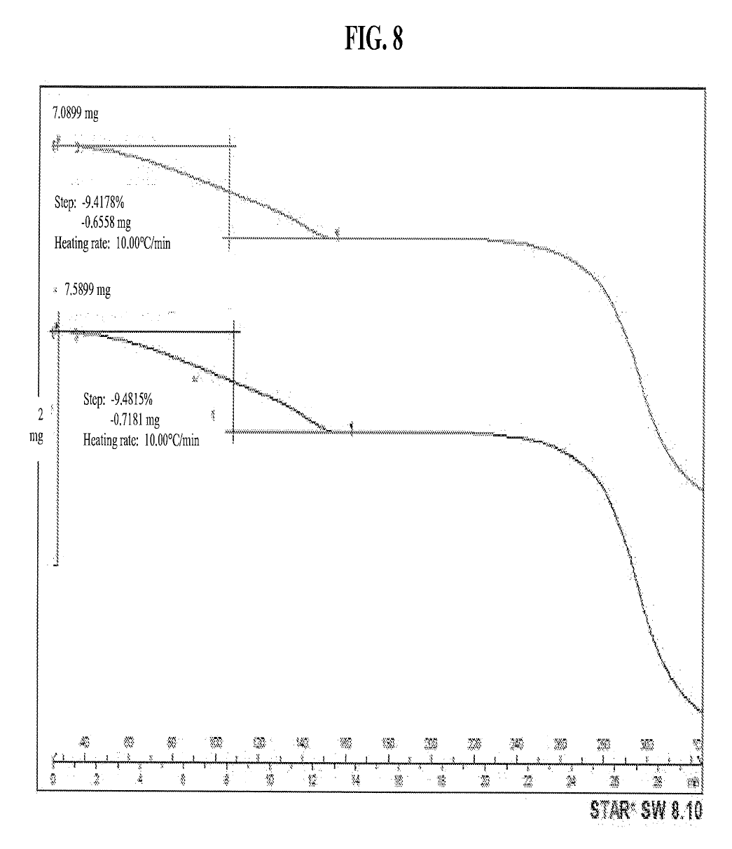

In certain embodiments, Form I of the mono-HCl salt of Compound A1 exhibits particular thermal characteristics. Representative thermal characteristics of Form I of the mono-HCl salt of Compound A1 are shown in FIG. 6, FIG. 7, and FIG. 8. The representative DSC thermogram presented in FIG. 6 comprises (1) an endothermic event with an onset temperature of about ambient temperature and a peak temperature of about 61° C.; and (2) an endothermic event with an onset temperature of about 137° C. and a peak temperature of about 140° C. Another representative DSC thermogram, presented in FIG. 7, comprises (1) an endothermic event with an onset temperature of about 38° C. and (2) an endothermic event with an onset temperature of about 143° C. Yet another representative DSC thermogram, presented in FIG. 7, comprises (1) an endothermic event with an onset temperature of about 45° C. and (2) an endothermic with an onset temperature of about 144° C. In certain embodiments, Form I of the mono-HCl salt of Compound A1 exhibits a DSC thermogram comprising an endothermic event between about ambient temperature and about 160° C. In certain embodiments, Form I of the mono-HCl salt of Compound A1 exhibits a DSC thermogram comprising one or more endothermic events with an onset temperature and/or peak temperature between about ambient temperature and about 120° C. In certain embodiments, Form I of the mono-HCl salt of Compound A1 exhibits a DSC thermogram comprising an endothermic event with an onset temperature and/or peak temperature between about 130° C. and about 160° C., between about 135° C. and about 155° C.; or between about 140° C. and about 150° C. In certain embodiments, Form I of the mono-HCl salt of Compound A1 exhibits a DSC thermogram comprising an endothermic event with an onset temperature and/or peak temperature at about 135° C., 136° C., 137° C., 138° C., 139° C., 140° C., 141° C., 142° C., 143° C., 144° C., 145° C., 146° C., 147° C., 148° C., 149° C., or 150° C.

The representative TGA thermogram presented in FIG. 6 comprises a mass loss of about 2.5% of the total mass of the sample upon heating from about ambient temperature to about 100° C. Another representative TGA thermogram, presented in FIG. 8, comprises a mass loss of about 9.4% of the total mass of the sample upon heating from about ambient temperature to about 150° C. Yet another representative TGA thermogram, presented in FIG. 8, comprises a mass loss of about 9.5% of the total mass of the sample upon heating from about ambient temperature to about 150° C. In certain embodiments, Form I of the mono-HCl salt of Compound A1 exhibits a TGA thermogram comprising a mass loss of between about 0% and about 15% of the total mass of the sample. In certain embodiments, Form I of the mono-HCl salt of Compound A1 exhibits a TGA thermogram comprising a mass loss of about 0%, about 1%, about 2%, about 3%, about 4%, about 5%, about 6%, about 7%, about 8%, about 9%, about 10%, about 11%, about 12%, about 13%, about 14%, or about 15% of the total mass of the sample when heated from about ambient temperature to about 150° C. In certain embodiments, the mass loss corresponds to a loss of solvent (such as, e.g., water and/or alcohol).

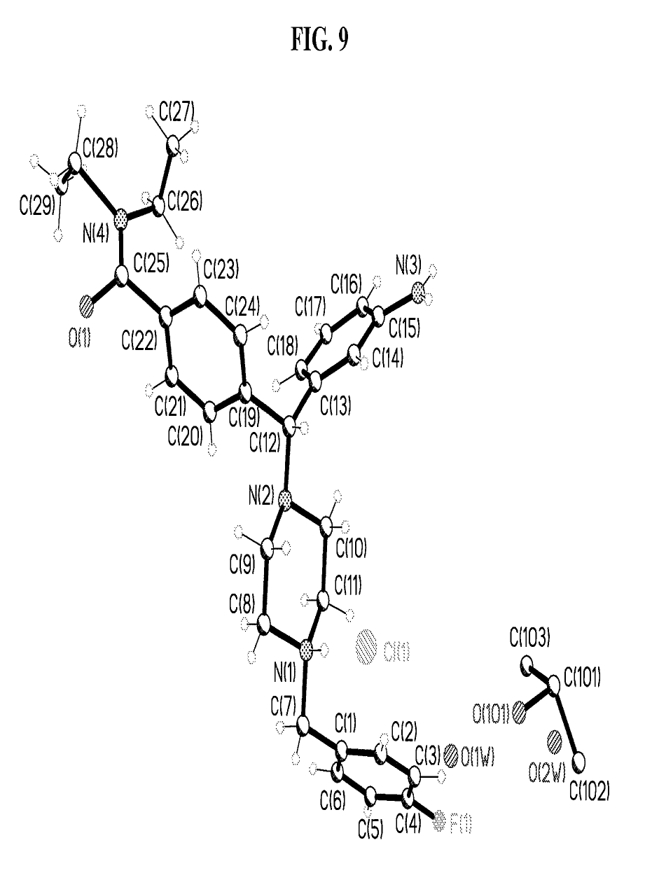

In certain embodiments, Form I of the mono-HCl salt of Compound A1 exhibits particular structural characteristics, as determined, e.g., by crystal structure analysis. A representative diagram corresponding to the asymmetric unit of a single crystal of Form I of the mono-HCl salt of Compound A1 isolated from a solution comprising isopropanol and water is shown in FIG. 9. In certain embodiments, Form I of the mono-HCl salt of Compound A1 is protonated at nitrogen N1 as indicated in the diagram. In certain embodiments, Form I of the mono-HCl of Compound A1 is unprotonated at nitrogens N2 and/or N4, as indicated in the diagram. In certain embodiments, Form I of the mono-HCl salt of Compound A1 crystallizes in an asymmetric unit comprising one cation of Compound A1, one chloride anion, two water molecules and one isopropyl alcohol molecule. In particular embodiments, Compound A1 crystallizes in an asymmetric unit as depicted in FIG. 9. In certain embodiments, Form I of the mono-HCl salt of Compound A1 has the following approximate unit cell parameters when measured at about 173 K: a=18.43 Å; b=18.43 Å; c=18.67 Å; α=90°; β=90°; γ=90°. In certain embodiments, Form I of the mono-HCl salt of Compound A1 has a unit cell volume of about 6344.2 cubic angstroms when measured at about 173 K. In certain embodiments, Form I of the mono-HCl salt of Compound A1 crystallizes in a tetragonal crystal system. In certain embodiments, Form I of the mono-HCl salt of Compound A1 crystallizes in the space group P4(3)2(1)2 with Z=8, where Z represents the number of asymmetric units per unit cell. In certain embodiments, Form I of the mono-HCl salt of Compound A1 has a density of about 1.198 Mg/m³ when measured at about 173 K. In certain embodiments, the crystal lattice of Form I of the mono-HCl salt comprises approximately 1 molar equivalent of isopropyl alcohol (IPA) per mole of Compound A1. In certain embodiments, the crystal lattice of Form I of the mono-HCl salt comprises approximately 1.5 molar equivalents of water per mole of Compound A1. In certain embodiments, Form I of the mono-HCl salt of Compound A1 is a sesquihydrate and/or a mono-IPA solvate.

In certain embodiments, Form I of the mono-HCl salt of Compound A1 comprises a stoichiometric amount (e.g., about 0.5 molar eq., about 1.0 molar eq., about 1.5 molar eq., about 2.0 molar eq., about 2.5 molar eq., or about 3.0 molar eq.) of one or more of the solvents (e.g., a solvent from which it is crystallized and/or a exposed via humidity exposure). In certain embodiments, Form I of the mono-HCl salt of Compound A1 comprises a stoichiometric amount (e.g., about 0.5 molar eq., about 1.0 molar eq., about 1.5 molar eq., about 2.0 molar eq., about 2.5 molar eq., or about 3.0 molar eq.) of water. In certain embodiments, Form I of the mono-HCl salt of Compound A1 comprises a non-stoichiometric amount of water.

In certain embodiments, Form I of the mono-HCl salt of Compound A1 is substantially chemically stable. For example, in certain embodiments, a sample of Form I of the mono-HCl salt is chemically stable (e.g., exhibits total organic impurities of less than about 10%, less than about 5%, less than about 4%, less than about 3%, less than about 2%, less than about 1%, less than about 0.5%, less than about 0.25%, or less than about 0.1%) upon storage at about 5° C. for 2 weeks and/or 4 weeks, with desiccant in a closed vial. In certain embodiments, a sample of Form I of the mono-HCl salt is chemically stable (e.g., exhibits total organic impurities of less than about 10%, less than about 5%, less than about 4%, less than about 3%, less than about 2%, less than about 1%, less than about 0.5%, less than about 0.25%, or less than about 0.1%) upon storage at about 40° C. and about 75% relative humidity (RH) for 2 weeks and/or 4 weeks, with or without desiccant. In certain embodiments, a sample of Form I of the mono-HCl salt is chemically stable (e.g., exhibits total organic impurities of less than about 10%, less than about 5%, less than about 4%, less than about 3%, less than about 2%, less than about 1%, less than about 0.5%, less than about 0.25%, or less than about 0.1%) upon storage at about 60° C. and about 80% RH after 2 weeks and/or after 4 weeks, with or without desiccant. In certain embodiments, a sample of Form I of the mono-HCl salt is chemically stable (e.g., exhibits total organic impurities of less than about 10%, less than about 5%, less than about 4%, less than about 3%, less than about 2%, less than about 1%, less than about 0.5%, less than about 0.25%, or less than about 0.1%) upon storage at about 80° C. for 2 weeks and/or 4 weeks, with or without desiccant. In certain embodiments, a sample stored “with desiccant” is stored in an open primary container (e.g., an uncapped vial), which is stored together with a desiccant (e.g., a 1 g SORB-IT® can) in a closed secondary container (e.g., a capped bottle), which is stored within the humidity chamber. In certain embodiments, a sample stored “without desiccant” is stored in an open primary container (e.g., an uncapped vial), which is stored in a covered secondary container (e.g., a bottle covered with TYVEK®), which is stored within the humidity chamber.

In certain embodiments, Form I of the mono-HCl salt of Compound A1 is substantially physically stable. For example, in certain embodiments, a sample of Form I of the mono-HCl salt is physically stable (e.g., does not undergo crystal form change as observed by XRPD analysis) upon storage for 1 week at about 25° C. and about 60% RH. In certain embodiments, a sample of Form I of the mono-HCl salt is physically stable (e.g., does not undergo crystal form change as observed by XRPD analysis) upon storage for 1 week at about 40° C. and about 75% RH.

In certain embodiments, Form I of the mono-HCl salt of Compound A1 is chemically pure. In certain embodiments, Form I of the mono-HCl salt of Compound A1 is physically pure. In certain embodiments, Form I of the mono-HCl salt of Compound A1 is substantially free of non-aqueous solvents. In certain embodiments, Form I of the mono-HCl salt of Compound A1 is a hydrate.

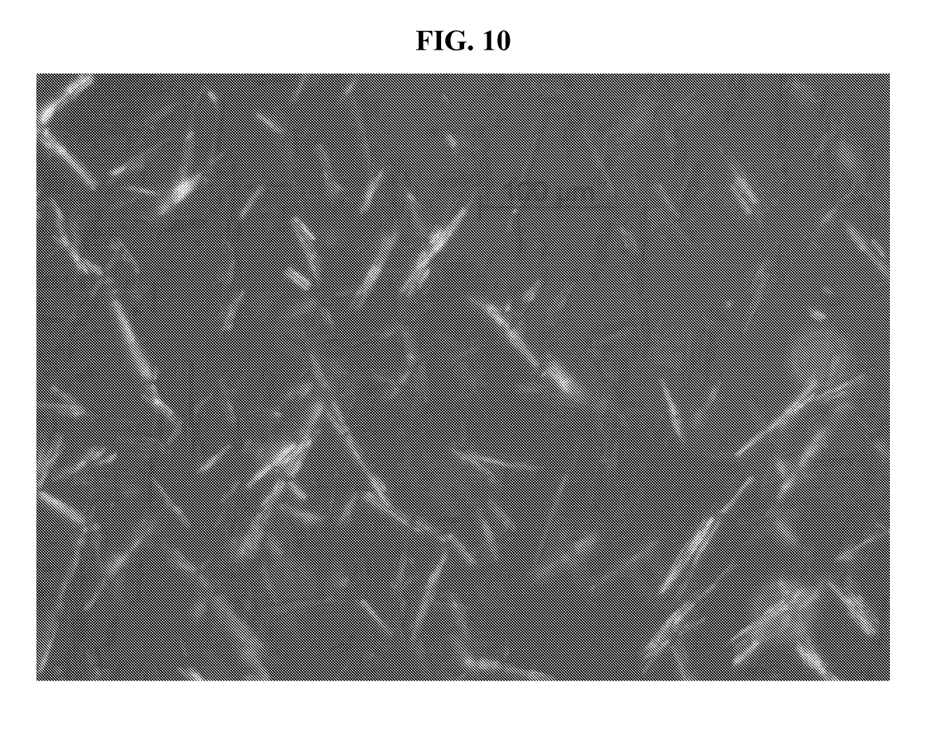

A representative polarized light micrograph corresponding to a sample of Form I of the mono-HCl salt of Compound A1 is shown in FIG. 10. In certain embodiments, a sample of Form I of the mono-HCl salt of Compound A1 contains birefringent particles comprising about 100%, about 90%, about 80%, about 70%, about 60%, or about 50% of the total number of particles in the sample. In certain embodiments, particles of Form I of the mono-HCl salt of Compound A1 are rod-shaped. In certain embodiments, particles of Form I of the mono-HCl salt of Compound A1 are needle-shaped. In certain embodiments, particles of the Form I of the mono-HCl salt of Compound A1 have an average particle size of about 200 μm, about 150 μm, about 100 μm, about 75 μm, about 50 μm, about 25 μm, about 20 μm, about 15 μm, about 10 μm, about 5 μm, about 1 μm, or less than about 1 μm.

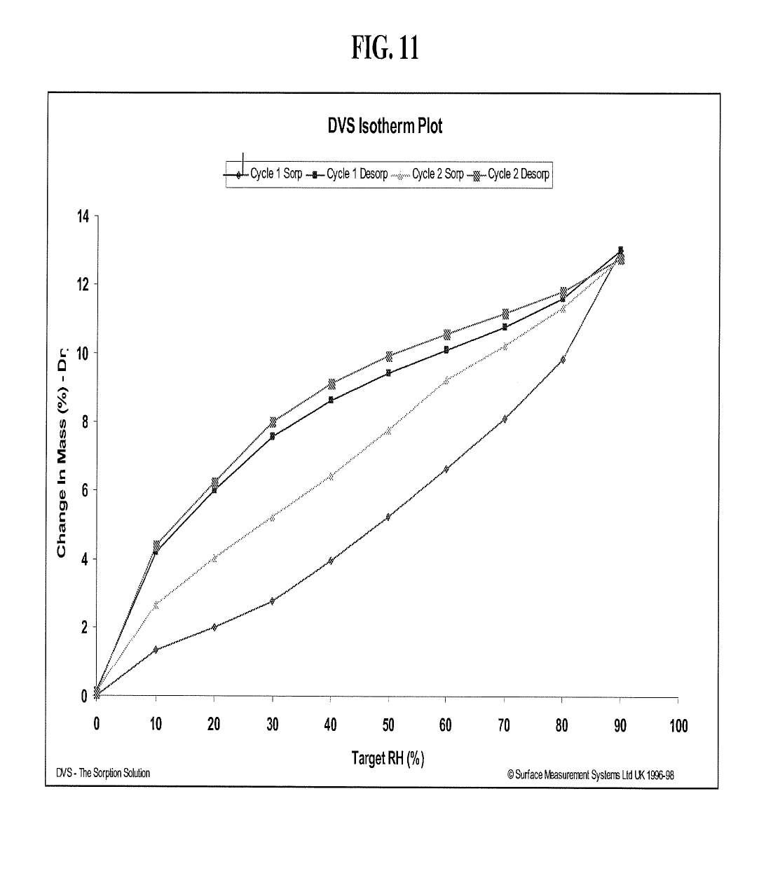

In certain embodiments, Form I of the mono-HCl salt of Compound A1 exhibits particular characteristics with respect to moisture sorption. For example, in certain embodiments, Form I of the mono-HCl salt of Compound A1 exhibits a moisture sorption profile matching the profile provided in FIG. 11. In certain embodiments, a sample of Form I of the mono-HCl salt of Compound A1 gains less than about 20%, less than about 19%, less than about 18%, less than about 17%, less than about 16%, less than about 15%, less than about 14%, less than about 13%, less than about 12%, less than about 11%, less than about 10%, less than about 9%, less than about 8%, less than about 7%, less than about 6%, or less than about 5% weight when increased from about 0% RH to about 90% RH at about ambient temperature. In certain embodiments, the weight gain is reversible upon decreasing from about 90% RH to about 0% RH at about ambient temperature.

In certain embodiments, Form I of the mono-HCl salt of Compound A1 exhibits particular characteristics with respect to solubility and dissolution. For example, in certain embodiments, Form I of the mono-HCl salt has a solubility of greater than about 41 mg/ml at ambient temperature in water with a pH of about 4.2. In certain embodiments, Form I of the mono-HCl salt has a solubility of greater than about 40 mg/ml at ambient temperature in water with a pH of about 3.3 (0.1 M phosphoric acid). In certain embodiments, Form I of the mono-HCl salt has a solubility of between about 15 mg/ml and 25 mg/ml (e.g., 23 mg/ml at 3 hr, 22 mg/ml at 24 hr) at ambient temperature in simulated gastric fluid with an initial pH of about 1.3. In certain embodiments, Form I of the mono-HCl salt has a solubility of between about 0.5 mg/ml and 2.5 mg/ml (e.g., 1.72 mg/ml at 3 hr, 0.83 mg/ml at 24 hr) at ambient temperature in fasted-state simulated intestinal fluid with an initial pH of about 6.51. In certain embodiments, Form I of the mono-HCl salt has a solubility of between about 2 mg/ml and 5 mg/ml (e.g., 3.40 mg/ml at 3 hr, 3.06 mg/ml at 24 hr) at ambient temperature in fed-state simulated intestinal fluid with an initial pH of about 5.03. In certain embodiments, Form I of the mono-HCl salt has a solubility of between about 0.05 mg/ml and 0.5 mg/ml (e.g., 0.29 mg/ml at 3 hr, 0.10 mg/ml at 24 hr) at ambient temperature in 0.1M phosphate buffer with an initial pH of about 7.5. In certain embodiments, Form I of the mono-HCl salt has an intrinsic dissolution rate (IDR) of about between 25 μg/min/cm² and 75 μg/min/cm² (e.g., 48.4 μg/min/cm²) at ambient temperature in fasted-state simulated intestinal fluid with an initial pH of about 6.51. In certain embodiments, Form I of the mono-HCl salt has an IDR of between about 75 μg/min/cm² 125 μg/min/cm² (e.g., 91.6 μg/min/cm²) at ambient temperature in fed-state simulated intestinal fluid with an initial pH of about 5.03. In certain embodiments, Form I of the mono-HCl salt at ambient temperature in simulated gastric fluid is too soluble to permit determination of its IDR in this medium.

In certain embodiments, Form I of the mono-HCl salt can be obtained from any suitable laboratory solvent, including, but not limited to, solvent systems comprising ethanol, isopropyl alcohol, isopropyl acetate, tert-butylmethylether, tetrahydrofuran, ethyl acetate, acetonitrile, water, dichloromethane, petroleum ether, toluene, acetone, or a mixture of two or more thereof. In certain embodiments, the solvent system comprises a common laboratory solvent, as known in the art. In certain embodiments, Form I of the mono-HCl salt of Compound A1 may be obtained by performing any three, four, five, six, seven, or eight of the following steps: (a) obtain a first solution comprising the free base of Compound A1; (b) obtain a second solution comprising hydrochloric acid; (c) heat the first solution to a temperature above ambient temperature; (d) admix the first solution and the second solution such that the resulting mixture comprises approximately one molar equivalent of hydrochloric acid per mole of Compound A1; (e) stir the mixture at a temperature above ambient temperature; (f) cool the mixture to a temperature approximately equal to or below ambient temperature; (g) isolate Form I of the mono-HCl salt of Compound A1; and (h) dry Form I of the mono-HCl salt.

In certain embodiments, the first solution in step (a) comprises ethanol, isopropyl alcohol, isopropyl acetate, tert-butylmethylether, tetrahydrofuran, water or a mixture of two or more thereof. In a particular embodiment, the first solution in step (a) comprises ethanol. In certain embodiments, the second solution in step (b) comprises ethanol, isopropyl alcohol, isopropyl acetate, tert-butylmethylether, tetrahydrofuran, water, or a mixture of two or more thereof. In a particular embodiment, the second solution in step (b) comprises isopropyl alcohol. In certain embodiments, the temperature in step (c) is above about 30° C., above about 40° C., above about 50° C., above about 60° C., or above about 70° C. In certain embodiments, the temperature in step (c) is about 35° C., about 40° C., about 45° C., about 50° C., about 55° C., about 60° C., about 65° C., about 70° C., about 75° C., or about 80° C. In certain embodiments, the resulting mixture in step (d) comprises about 0.75, about 0.80, about 0.85, about 0.90, about 0.95, about 1.00, about 1.05, about 1.10, about 1.15, about 1.20, or about 1.25 molar equivalents of hydrochloric acid per mole of Compound A1. In certain embodiments, the temperature in step (e) is above about 30° C., above about 40° C., above about 50° C., above about 60° C., or above about 70° C. In certain embodiments, the temperature in step (e) is about 35° C., about 40° C., about 45° C., about 50° C., about 55° C., about 60° C., about 65° C., about 70° C., about 75° C., or about 80° C. In certain embodiments, the temperature in step (f) is about 25° C., about 20° C., about 15° C., about 10° C., about 5° C., about 0° C., or less than 0° C. In certain embodiments, the isolation in step (g) comprises suction filtration. In certain embodiments, the drying in step (h) comprises vacuum drying. In certain embodiments, the drying in step (h) comprises drying at or below about 70° C., at or below about 60° C., at or below about 50° C., at or about 40° C., at below about 30° C., or at about ambient temperature.

In certain embodiments, Form I of the mono-HCl salt can be obtained by contacting the amorphous form of the mono-HCl salt with water and/or water vapor. In certain embodiments, Form I of the mono-HCl salt can be obtained by exposing the amorphous form of the mono-HCl salt to a high relative humidity (e.g., greater than about 50%, RH greater than about 60% RH, greater than about 70% RH, greater than about 80% RH, or greater than about 90% RH). Optionally, the humidity exposure can be performed at a temperature above ambient temperature. For example, in specific embodiments, Form I of the mono-HCl salt can be obtained by exposing the amorphous form of the mono-HCl salt to about 75% RH at about 40° C.

In particular embodiments, Form I of the mono-HCl salt of Compound A1 showed advantageous properties including properties relating to, e.g., crystallinity, water uptake (e.g., hygroscopicity), low levels of residual non-aqueous solvent, and chemical stability.

### Amorphous Form of the Mono-HCl Salt of Compound A1

Certain embodiments herein provide an amorphous form of the mono-HCl salt of Compound A1.

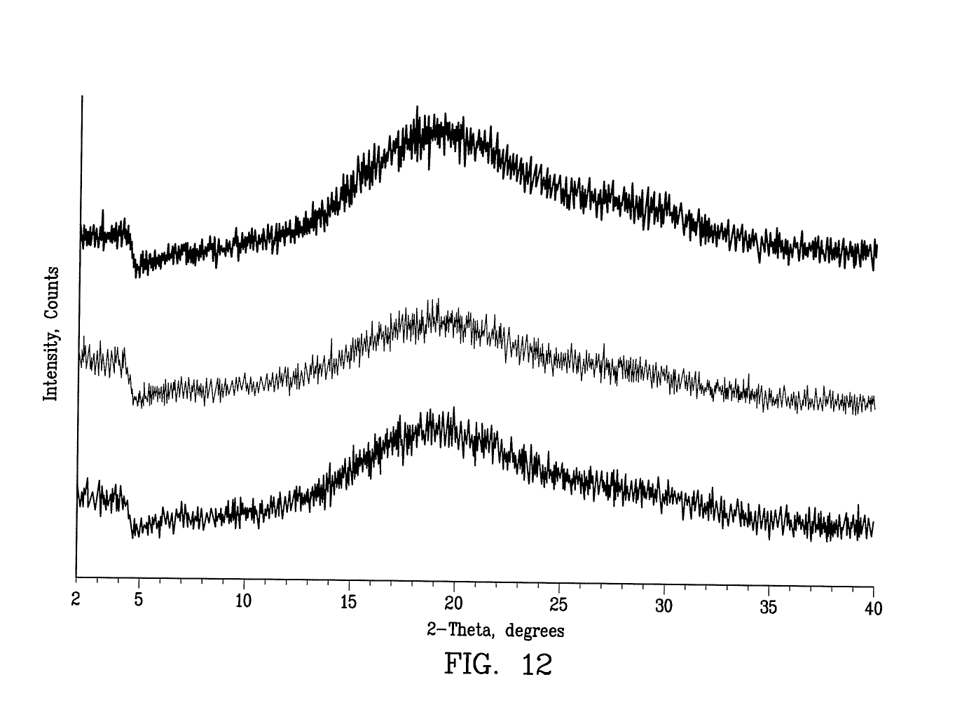

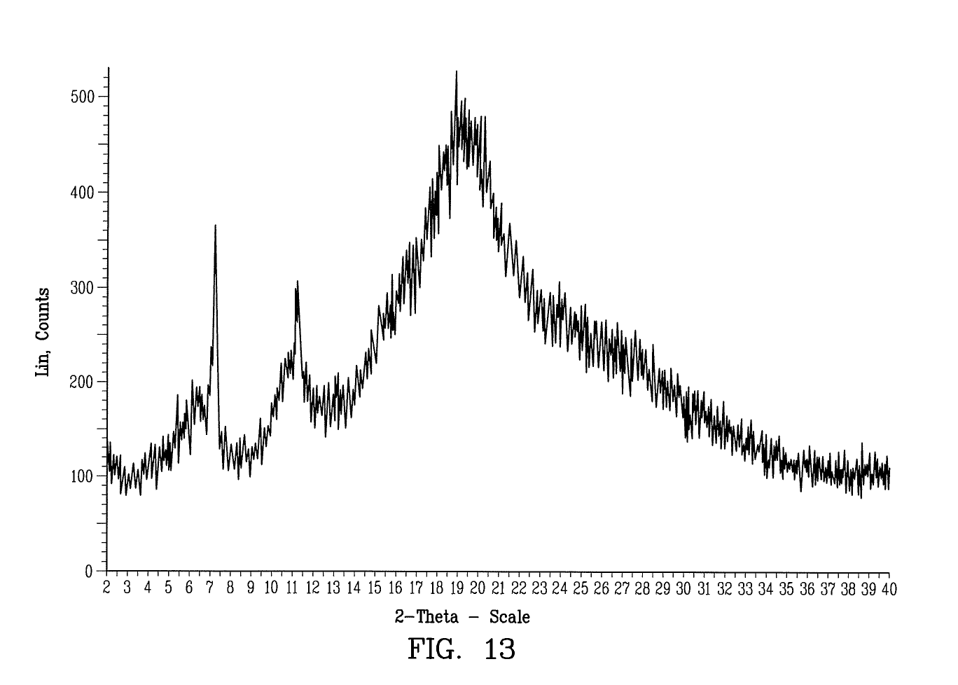

In certain embodiments, the amorphous form of the mono-HCl salt of Compound A1 exhibits particular structural characteristics, as determined, e.g., by diffraction analysis. Representative XRPD patterns of the amorphous form of the mono-HCl salt of Compound A1 are provided in FIG. 12 and FIG. 13. In certain embodiments, the amorphous form of the mono-HCl salt is characterized by an XRPD pattern with no peaks (reflections) indicative of crystal lattice planes and/or long range order. In certain embodiments, the amorphous form of the mono-HCl salt is characterized by an XRPD pattern with fewer than 10, fewer than 9, fewer than 8, fewer than 7, fewer than 6, fewer than 5, fewer than 4, fewer than 3, or fewer than 2 peaks (reflections) indicative of crystal lattice planes and/or long range order. In certain embodiments, the amorphous form of the mono-HCl salt has an XRPD pattern comprising a halo indicative of amorphous material. In particular embodiments, the halo has a maximum between about 12 and 25 degrees 2θ, between about 14 and 23 degrees 2θ, between about 16 and 21 degrees 2θ, or between about 17 and 20 degrees 2θ. In certain embodiments, the amorphous form of the mono-HCl salt of Compound A1 is characterized by an XRPD pattern which matches the patterns exhibited in FIG. 12. In certain embodiments, the amorphous form of the mono-HCl salt of Compound A1 is characterized by an XRPD pattern which matches the pattern exhibited in FIG. 13.

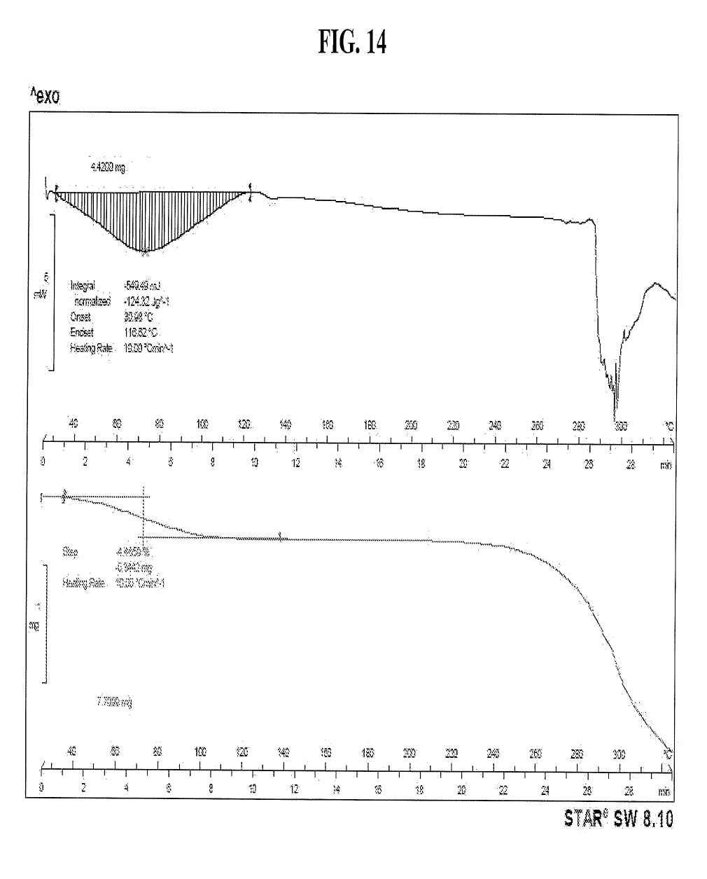

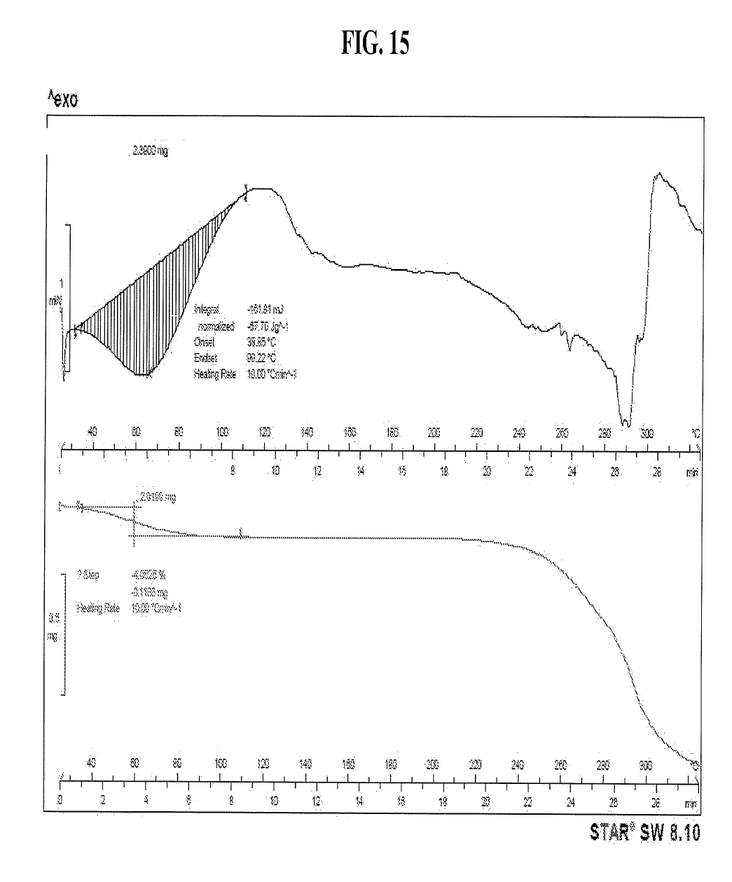

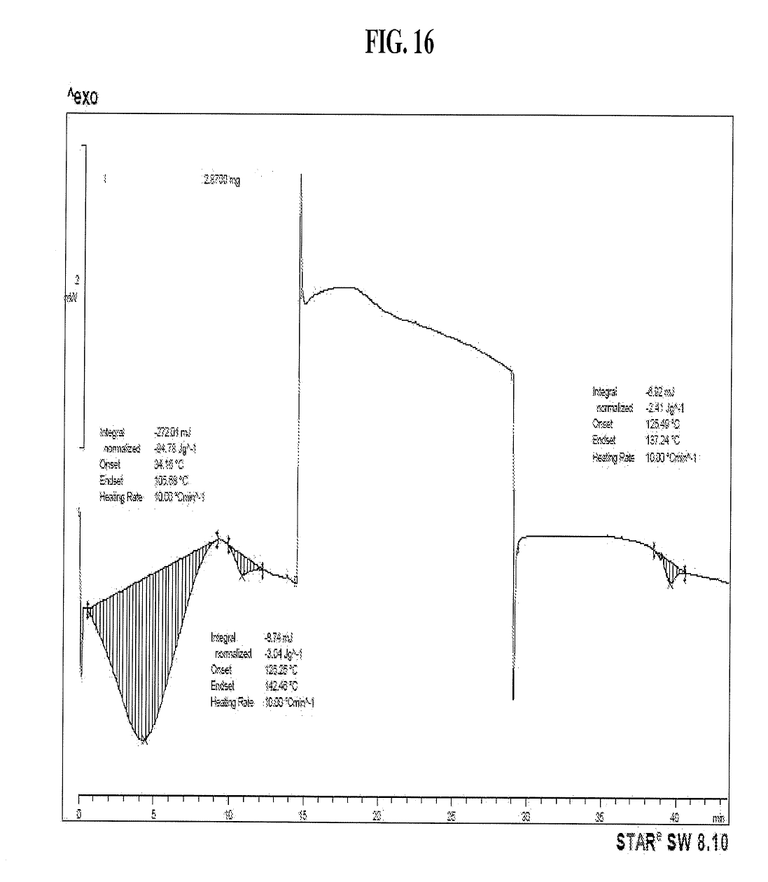

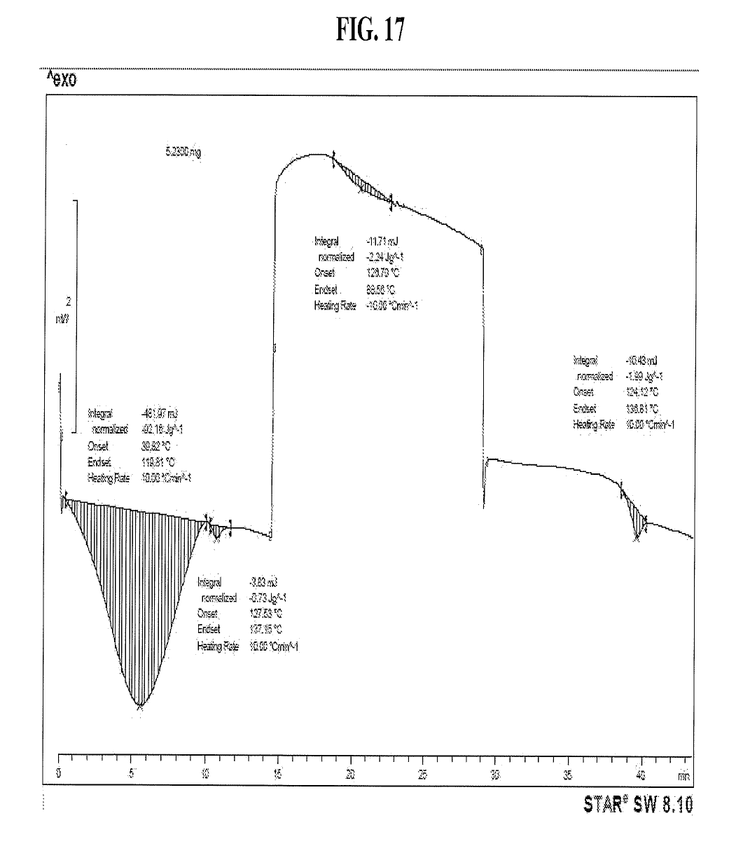

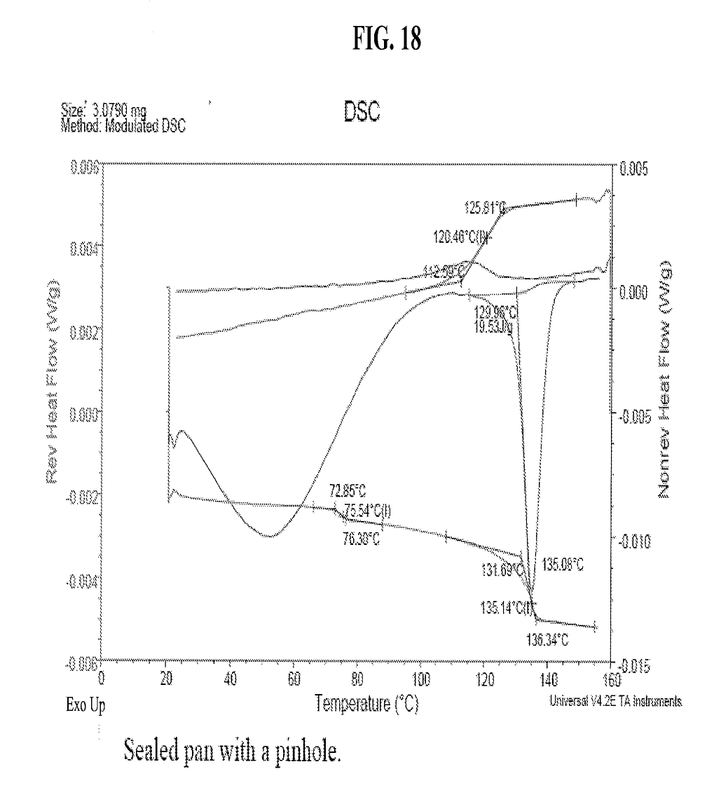

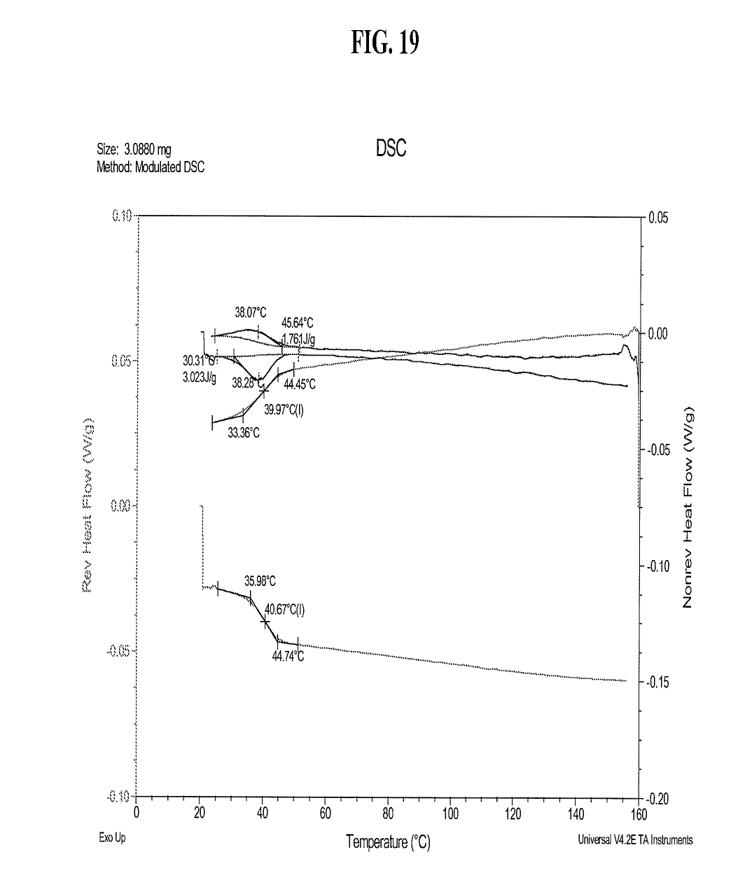

In certain embodiments, the amorphous form of the mono-HCl salt of Compound A1 exhibits particular thermal characteristics. Representative thermal characteristics of the amorphous form of the mono-HCl salt of Compound A1 are shown in FIG. 14, FIG. 15, FIG. 16, FIG. 17, FIG. 18, and FIG. 19. The representative DSC thermograms presented in FIG. 16 and FIG. 17 comprise an endothermic event between about ambient temperature and about 130° C. The representative TGA thermograms presented in FIG. 16 and FIG. 17 comprise a mass loss between about 0% and about 10%, or between about 3% and about 5% (e.g., 4.1% and 4.5%), of the total mass of the sample upon heating from ambient temperature to about 130° C. The representative Temperature-Cycling DSC thermograms, presented in FIG. 16 and FIG. 17, comprise, during an initial heating stage (from about 25° C. to about 156° C. at about 10° C./min), (1) an endothermic event between about ambient temperature and about 125° C.; and, optionally, (2) an endothermic event between about 125° C. and about 150° C.; and further comprise, during a second heating stage (from about 25° C. to about 156° C. at about 10° C./min), (3) an endothermic event between about 110° C. and about 140° C. (e.g., with onset temperatures of about 125° C. and 124° C.). In particular embodiments, the two heating stages are separated by a cooling stage with a cooling rate of about −10° C./min. In certain embodiments, the amorphous form of the mono-HCl salt exhibits a DSC thermogram which matches the representative DSC thermogram in FIG. 14 and/or FIG. 15. In certain embodiments, the amorphous form of the mono-HCl salt exhibits a TGA thermogram which matches the representative TGA thermogram in FIG. 14 and/or FIG. 15. In certain embodiments, the amorphous form of the mono-HCl salt exhibits a Temperature-Cycling DSC thermogram which matches the representative Temperature-Cycling DSC thermogram in FIG. 16 and/or FIG. 17. In certain embodiments, the amorphous form of the mono-HCl salt exhibits a Modulated DSC thermogram which matches the representative Modulated DSC thermogram in FIG. 16 and/or FIG. 17.

Representative Modulated DSC thermograms are presented in FIG. 18 and FIG. 19. In certain embodiments, the amorphous form of the mono-HCl salt exhibits a glass transition temperature (Tg) between about 30° C. and about 130° C. In particular embodiments, the glass transition temperature is about 30° C., about 35° C., about 40° C., about 45° C., about 50° C., about 55° C., about 60° C., about 65° C., about 70° C., about 75° C., about 80° C., about 85° C., about 90° C., about 95° C., about 100° C., about 105° C., about 110° C., about 115° C., about 120° C., about 125° C., or about 130° C. In certain embodiments the glass transition temperature of a sample of the amorphous form of the mono-HCl salt is affected by the water content of the sample (e.g., increasing water content corresponds to decreasing Tg). For example, in certain embodiments, a sample with a water content of about 3.2% exhibits a Tg of about 72.1° C.; a sample with a water content of about 5.0% exhibits a Tg of about 63.2° C.; a sample with a water content of about 6.2% exhibits a Tg of about 56.5° C.; a sample with a water content of about 8.2% exhibits a Tg of about 44.2° C.; and/or a sample with a water content of about 9.5% exhibits a Tg of about 33.0° C. In certain embodiments, the glass transition temperature is measured using modulated DSC.

In certain embodiments, the amorphous form of the mono-HCl salt of Compound A1 is substantially chemically stable. For example, in certain embodiments, a sample of the amorphous form of the mono-HCl salt is chemically stable (e.g., exhibits total organic impurities of less than about 10%, less than about 5%, less than about 4%, less than about 3%, less than about 2%, less than about 1%, less than about 0.5%, less than about 0.25%, or less than about 0.1%) upon storage at about 40° C., about 50° C., and/or about 60° C. at ambient relative humidity (RH) for 2 weeks. In certain embodiments, a sample of the amorphous form of the mono-HCl salt is chemically stable (e.g., exhibits total organic impurities of less than about 10%, less than about 5%, less than about 4%, less than about 3%, less than about 2%, less than about 1%, less than about 0.5%, less than about 0.25%, or less than about 0.1%) upon storage after 2 weeks and after 4 weeks at about 25° C./60% RH with desiccant, about 25° C./60% RH without desiccant, about 40° C./75% RH with desiccant, and/or about 40° C./75% RH without desiccant. In certain embodiments, a sample stored “with desiccant” is stored in an open primary container (e.g., an uncapped vial), which is stored together with a desiccant (e.g., a 1 g SORB-IT® can) in a closed secondary container (e.g., a capped bottle), which is stored within the humidity chamber. In certain embodiments, a sample stored “without desiccant” is stored in an open primary container (e.g., an uncapped vial), which is stored in a covered secondary container (e.g., a bottle covered with TYVEK®), which is stored within the humidity chamber.

In certain embodiments, the amorphous form of the mono-HCl salt of Compound A1 is substantially physically stable (e.g., does not exhibit deliquescence, does not undergo crystallization as observed by XRPD analysis, and/or does not exhibit morphological change as observed by polarized light microscopy). For example, in certain embodiments, a sample of the amorphous form of the mono-HCl salt is physically stable upon storage for about 17 days at about 25° C. and about 60% RH. In certain embodiments, a sample of the amorphous form of the mono-HCl salt is physically stable upon storage for 1 week at about 40° C. and about 75% RH. In certain embodiments, a sample of the amorphous form of the mono-HCl salt is physically stable upon storage at ambient temperature for about 3 days at about 23% RH, about 43% RH, about 54% RH, and/or about 76% RH.

In certain embodiments, the amorphous form of the mono-HCl salt of Compound A1 is chemically pure. In certain embodiments, the amorphous form of the mono-HCl salt of Compound A1 is physically pure.

In particular embodiments, a sample of the amorphous form of the mono-HCl salt of Compound A1 is substantially amorphous. In certain embodiments, a sample of the amorphous form of the mono-HCl salt of Compound A1 contains birefringent particles comprising less than about 50%, less than about 40%, less than about 30%, less than about 20%, less than about 10%, less than about 5%, less than about 4%, less than about 3%, less than about 2%, or less than about 1% of the total number of particles in the sample. In certain embodiments, particles of the amorphous form of the mono-HCl salt of Compound A1 are rod-shaped and/or needle-shaped (e.g., when the particles are obtained via dehydration). In certain embodiments, particles of the amorphous form of the mono-HCl salt of Compound A1 are irregularly shaped (e.g., when the particles are obtained via evaporation). In certain embodiments, particles of the amorphous form of the mono-HCl salt of Compound A1 are spherically shaped (e.g., when the particles are obtained via spray drying). In certain embodiments, particles of the amorphous form of the mono-HCl salt of Compound A1 have an average particle size of about 200 μm, about 150 μm, about 100 μm, about 75 μm, about 50 μm, about 25 μm, about 20 μm, about 15 μm, about 10 μm, about 5 μm, about 1 μm, or less than about 1 μm.

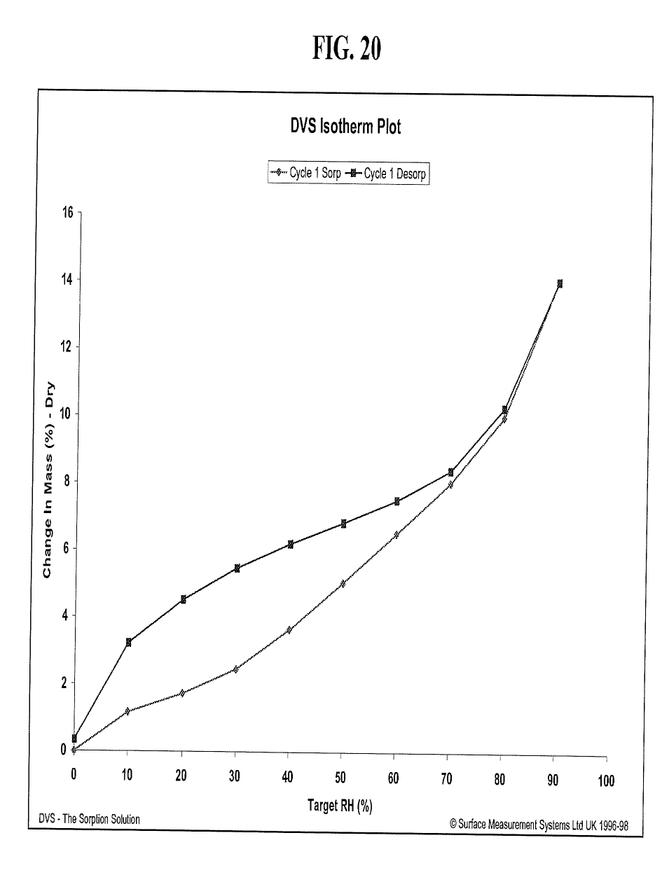

In certain embodiments, the amorphous form of the mono-HCl salt of Compound A1 exhibits particular characteristics with respect to moisture sorption. For example, in certain embodiments, the amorphous form of the mono-HCl salt of Compound A1 exhibits a moisture sorption profile matching the profile provided in FIG. 20. In certain embodiments, a sample of the amorphous form of the mono-HCl salt of Compound A1 gains less than about 20%, less than about 19%, less than about 18%, less than about 17%, less than about 16%, less than about 15%, less than about 14%, less than about 13%, less than about 12%, less than about 11%, less than about 10%, less than about 9%, less than about 8%, less than about 7%, less than about 6%, or less than about 5% weight when increased from about 0% RH to about 90% RH at about ambient temperature. In certain embodiments, the weight gain is reversible upon decreasing from about 90% RH to about 0% RH at about ambient temperature. In certain embodiments, the amorphous form of the mono-HCl salt is substantially physically stable when subjected to the moisture sorption/desorption program (e.g., does not exhibit deliquescence, does not undergo crystallization, and/or does not exhibit morphological change as observed by polarized light microscopy).

In certain embodiments, the amorphous form of the mono-HCl salt of Compound A1 exhibits particular characteristics with respect to excipient compatibility. For example, in certain embodiments, the amorphous form of the mono-HCl salt of Compound A1 exhibits acceptable chemical stability (e.g., exhibits less than 3%, less than 2%, less than 1%, less than 0.75%, less than 0.5%, less than 0.25%, less than 0.1%, or about 0% chemical degradation) when formulated together in a pharmaceutical composition comprising one or more of the following excipients: microcrystalline cellulose (e.g., Avicel® PH 113); mannitol (e.g., Pearlitol® SD 200); hydroxypropyl cellulose (e.g., L-HPC HL-11); magnesium stearate; polyvinylpyrrolidone (e.g., A-TAB®); dicalcium phosphate (e.g., Polyplasdone® XL 10). In certain embodiments, the amorphous form of the mono-HCl salt of Compound A1 exhibits acceptable chemical stability (e.g., exhibits less than 3%, less than 2%, less than 1%, less than 0.75%, less than 0.5%, less than 0.25%, less than 0.1%, or about 0% chemical degradation) when formulated together in a pharmaceutical composition comprising one or more of the aforementioned excipients and stored at one or more of the following conditions: 2 weeks at 25° C.; 4 weeks at 25° C.; 2 weeks at 40° C.; 4 weeks at 40° C.; 12 weeks at 40° C.; 2 weeks at 40° C. and 75% RH; 4 weeks at 40° C. and 75% RH. In certain embodiments, a sample of the amorphous form of the mono-HCl salt of Compound A1 gains less than about 20%, less than about 19%, less than about 18%, less than about 17%, less than about 16%, less than about 15%, less than about 14%, less than about 13%, less than about 12%, less than about 11%, less than about 10%, less than about 9%, less than about 8%, less than about 7%, less than about 6%, or less than about 5% weight when increased from about 0% RH to about 90% RH at about ambient temperature. In certain embodiments, the weight gain is reversible upon decreasing from about 90% RH to about 0% RH at about ambient temperature. In certain embodiments, the amorphous form of the mono-HCl salt is substantially physically stable when subjected to the moisture sorption/desorption program (e.g., does not exhibit deliquescence, does not undergo crystallization, and/or does not exhibit morphological change as observed by polarized light microscopy).

In certain embodiments, the amorphous form of the mono-HCl salt of Compound A1 can be obtained from any suitable laboratory solvent, including, but not limited to, solvent systems comprising water or an alcohol (e.g., methanol). In certain embodiments, the amorphous form of the mono-HCl salt is prepared by a procedure comprising spray drying. In certain embodiments, the spray drying procedure comprises one, two, or three of the following steps: (1) dissolving a mono-HCl salt of Compound A1 in a solvent system to form a solution; (2) spray drying the solution to form the amorphous form of the mono-HCl salt; and (3) drying the amorphous form of the mono-HCl salt. In certain embodiments, the solvent system comprises methanol. In certain embodiments, the solution is approximately a 10% w/v solution. In certain embodiments, the solution is approximately a 10% w/v methanol solution. In certain embodiments, warming is required (e.g., to about 35° C.) to dissolve fully the solids in the solvent. In certain embodiments, the outlet temperature for the spray drying is between about 90° C. and about 65° C. (e.g., between about 85° C. and about 71° C.; between about 79° C. and about 71° C.; or about 80° C.). In certain embodiments, the yield following spray drying is greater than about 10%, greater than about 20%, greater than about 30%, greater than about 40%, greater than about 50%, greater than about 60%, greater than about 70%, greater than about 80%, greater than about 90%, greater than about 95%, or greater than about 99% (in particular embodiments, the yield is calculated by excluding filter recovery). In certain embodiments, no glassing occurs following spray drying. In certain embodiments, the step (3) drying step comprises drying in a vacuum. In certain embodiments, the step (3) drying step comprises drying with desiccant. In certain embodiments, the step (3) drying step comprises drying at a temperature of about 40° C. In certain embodiments, the step (3) drying step results in reduced residual solvent levels (e.g., less than 1.5% w/w, less than 1.25% w/w, less than 1.0% w/w, less than 0.9% w/w, less than 0.8% w/w, less than 0.7% w/w, less than 0.6% w/w, less than 0.5% w/w, less than 0.4% w/w, less than 0.3% w/w, less than 0.2% w/w, less than 0.1% w/w, less than 0.09% w/w, less than 0.8% w/w, less than 0.07% w/w, less than 0.06% w/w, less than 0.05% w/w, less than 0.04% w/w, less than 0.03% w/w, less than 0.02% w/w, or less than 0.01% w/w) in the amorphous form of the mono-HCl salt of Compound A1. In certain embodiments, the residual solvent comprises methanol, ethanol, isopropanol, and/or water. In certain embodiments, samples of the amorphous form of the mono-HCl salt obtained following spray drying are substantially amorphous.

In certain embodiments, the amorphous form of the mono-HCl salt is prepared by a procedure comprising vacuum drying. In certain embodiments, the vacuum drying procedure comprises drying a substantially crystalline sample of the mono-HCl salt of Compound A1 under vacuum (e.g., about 700 Torr, about 600 Torr, about 500 Torr, about 400 Torr, about 300 Torr, about 200 Torr, about 100 Torr, about 80 Torr, about 60 Torr, about 40 Torr, about 20 Torr, about 10 Torr, about 5 Torr, about 1 Torr, about 0.75 Torr, about 0.5 Torr, about 0.25 Torr, about 0.1 Torr, about 0.01 Torr, or less than about 0.01 Torr), and isolating the amorphous mono-HCl salt. In certain embodiments, the drying is performed at about ambient temperature. In certain embodiments, the drying is performed at or above about 30° C., about 40° C., about 50° C., about 60° C., about 70° C., about 80° C., about 90° C., or about 100° C. In a particular embodiment, the drying is performed at about 80° C. In certain embodiments, the vacuum drying results in reduced residual solvent levels (e.g., less than 1.5% w/w, less than 1.25% w/w, less than 1.0% w/w, less than 0.9% w/w, less than 0.8% w/w, less than 0.7% w/w, less than 0.6% w/w, less than 0.5% w/w, less than 0.4% w/w, less than 0.3% w/w, less than 0.2% w/w, less than 0.1% w/w, less than 0.09% w/w, less than 0.8% w/w, less than 0.07% w/w, less than 0.06% w/w, less than 0.05% w/w, less than 0.04% w/w, less than 0.03% w/w, less than 0.02% w/w, or less than 0.01% w/w) in the amorphous form of the mono-HCl salt of Compound A1. In certain embodiments, the residual solvent comprises methanol, ethanol, isopropanol, and/or water. In certain embodiments, samples of the amorphous form of the mono-HCl salt obtained following vacuum drying are substantially amorphous.

In particular embodiments, the amorphous form of the mono-HCl salt of Compound A1 showed advantageous properties including properties relating to, e.g., thermal properties (e.g., high Tg), physical stability, chemical stability, excipient compatibility, water uptake (e.g., hygroscopicity), solubility, and dissolution.

### Tri-HCl Salt of Compound A1

As used herein, a “tri-HCl salt” or “trihydrochloride salt” of Compound A1 is a salt which comprises approximately 3 molar equivalents of chloride ion per mole of Compound A1. In specific embodiments, a tri-HCl salt of Compound A1 comprises about 2.75, about 2.80, about 2.85, about 2.90, about 2.95, about 3.00, about 3.05, about 3.10, about 3.15, about 3.20, or about 3.25 molar equivalents of chloride ion per mole of Compound A1.

### Amorphous Form of the Tri-HCl Salt of Compound A1

Certain embodiments herein provide an amorphous form of the Tri-HCl salt of Compound A1.

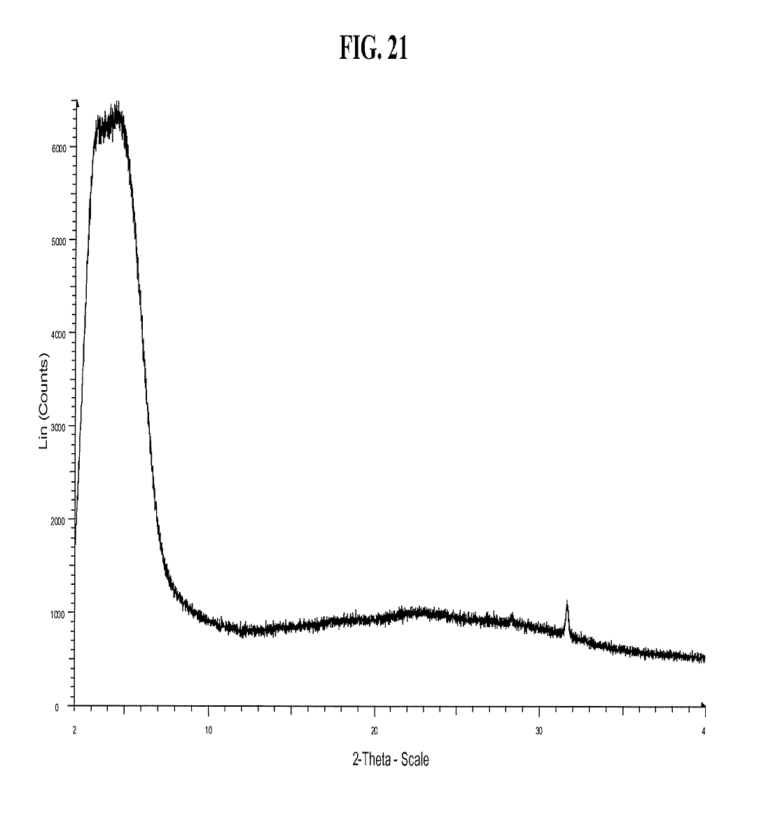

In certain embodiments, the amorphous form of the tri-HCl salt of Compound A1 exhibits particular structural characteristics, as determined, e.g., by diffraction analysis. A representative XRPD pattern of the amorphous form of the tri-HCl salt of Compound A1 is provided in FIG. 21. In certain embodiments, the amorphous form of the tri-HCl salt is characterized by an XRPD pattern with no peaks (reflections) indicative of crystal lattice planes and/or long range order. In certain embodiments, the amorphous form of the tri-HCl salt is characterized by an XRPD pattern with fewer than 10, fewer than 9, fewer than 8, fewer than 7, fewer than 6, fewer than 5, fewer than 4, fewer than 3, or fewer than 2 peaks (reflections) indicative of crystal lattice planes and/or long range order. In certain embodiments, the amorphous form of the tri-HCl salt of Compound A1 is characterized by an XRPD pattern which matches the pattern exhibited in FIG. 21.

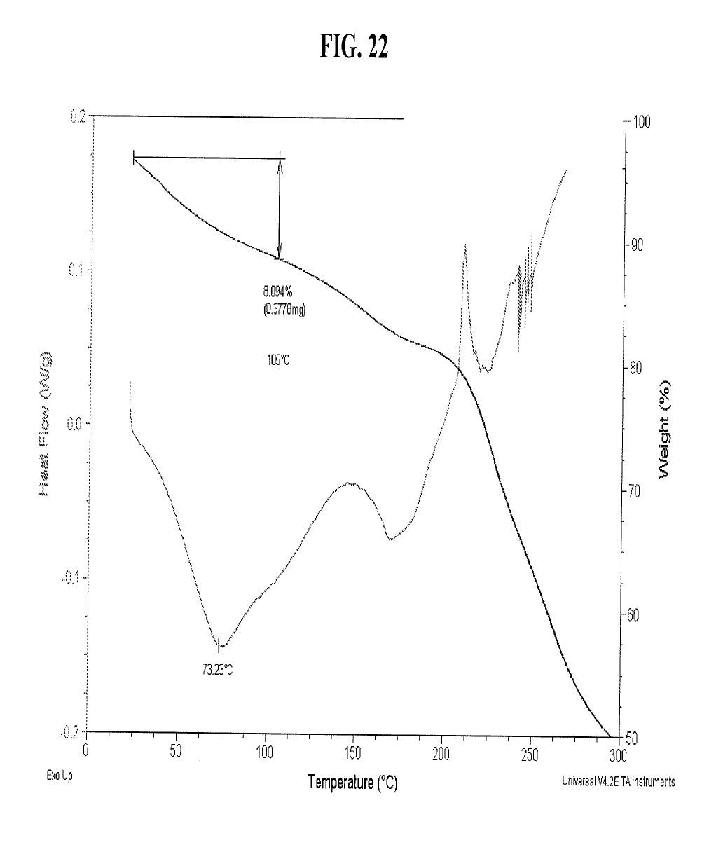

In certain embodiments, the amorphous form of the tri-HCl salt of Compound A1 exhibits particular thermal characteristics. Representative thermal characteristics of the amorphous form of the tri-HCl salt of Compound A1 are shown in FIG. 22. The representative DSC thermogram presented in FIG. 22 comprises an endothermic event between about ambient temperature and about 150° C. The representative TGA thermogram presented in FIG. 22 comprises a mass loss between about 0% and about 20% (e.g., 8%) of the total mass of the sample upon heating from ambient temperature to about 105° C. In certain embodiments, the amorphous form of the tri-HCl salt exhibits a DSC thermogram which matches the representative DSC thermogram in FIG. 22. In certain embodiments, the amorphous form of the tri-HCl salt exhibits a TGA thermogram which matches the representative TGA thermogram in FIG. 22.

In certain embodiments, the amorphous form of the tri-HCl salt of Compound A1 exhibits characteristic chemical stability parameters. For example, in certain embodiments, a sample of the amorphous form of the tri-HCl salt exhibits a total chemical purity of about 95%, about 94%, about 93%, about 92%, about 91%, about 90%, about 89%, about 88%, about 87%, or about 86% upon storage at about 60° C. and about 80% RH for 2 weeks and/or 4 weeks. In certain embodiments, a sample of the amorphous form of the tri-HCl salt exhibits a total chemical purity of about 95%, about 94%, about 93%, about 92%, about 91%, about 90%, about 89%, or about 88% upon storage at about 80° C. for 2 weeks and/or 4 weeks.

In certain embodiments, the amorphous form of the tri-HCl salt of Compound A1 is substantially physically stable (e.g., does not exhibit deliquescence, does not undergo crystallization as observed by XRPD analysis, and/or does not exhibit morphological change as observed by polarized light microscopy). For example, in certain embodiments, a sample of the amorphous form of the tri-HCl salt is physically stable upon storage for about 17 days at about 25° C. and about 60% RH. In certain embodiments, a sample of the amorphous form of the tri-HCl salt is physically stable upon storage for 1 week at about 40° C. and about 75% RH. In certain embodiments, a sample of the amorphous form of the tri-HCl salt is physically stable upon storage at ambient temperature for about 3 days at about 23% RH, about 43% RH, about 54% RH, and/or about 76% RH.

In certain embodiments, a sample of the tri-HCl salt of Compound A1 is substantially amorphous. In certain embodiments, a sample of the amorphous form of the tri-HCl salt of Compound A1 contains birefringent particles comprising less than about 50%, less than about 40%, less than about 30%, less than about 20%, less than about 10%, less than about 5%, less than about 4%, less than about 3%, less than about 2%, or less than about 1% of the total number of particles in the sample. In certain embodiments, particles of the amorphous form of the tri-HCl salt of Compound A1 have an average particle size of about 1,000 μm, 750 μm, 500 μm, 200 μm, about 150 μm, about 100 μm, about 75 μm, about 50 μm, about 25 μm, about 20 μm, about 15 μm, about 10 μm, about 5 μm, about 1 μm, or less than about 1 μm.

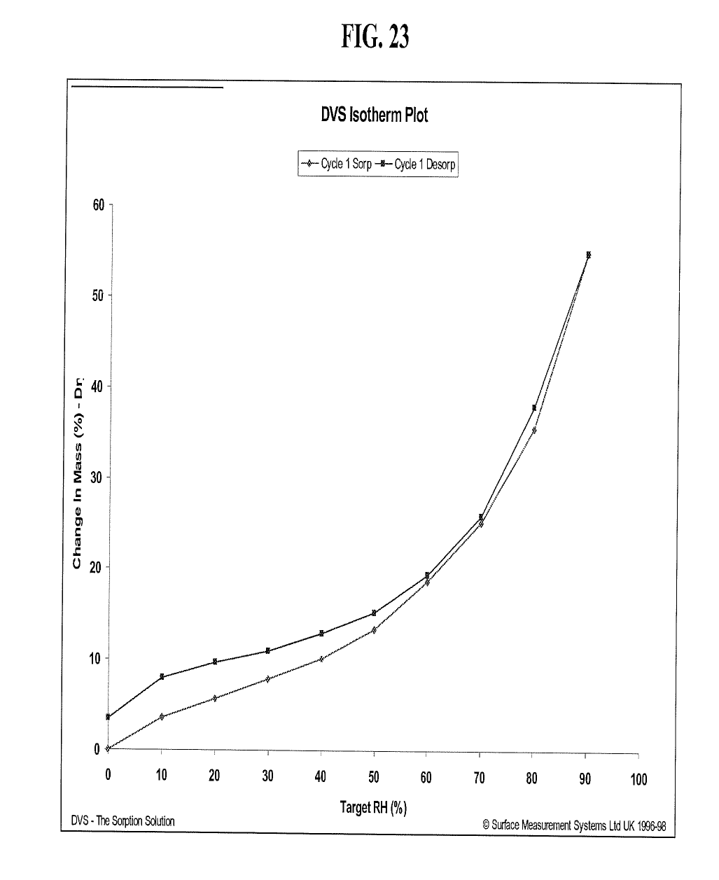

In certain embodiments, the amorphous form of the tri-HCl salt of Compound A1 exhibits particular characteristics with respect to moisture sorption. For example, in certain embodiments, the amorphous form of the tri-HCl salt of Compound A1 exhibits a moisture sorption profile matching the profile provided in FIG. 23. In certain embodiments, a sample of the amorphous form of the tri-HCl salt of Compound A1 gains less than about 70%, less than about 60%, less than about 50%, less than about 40%, less than about 30%, less than about 20%, less than about 10%, less than about 8%, less than about 6%, less than about 4%, less than about 2%, or less than about 1% weight when increased from about 0% RH to about 90% RH at about ambient temperature. In certain embodiments, the weight gain is reversible upon decreasing from about 90% RH to about 0% RH at about ambient temperature. In certain embodiments, the amorphous form of the tri-HCl salt is substantially physically stable when subjected to the moisture sorption/desorption program (e.g., does not exhibit substantial deliquescence, does not undergo substantial crystallization as observed by XRPD analysis, and/or does not exhibit substantial morphological change as observed by polarized light microscopy).

In certain embodiments, the amorphous form of the tri-HCl salt of Compound A1 can be obtained from any suitable laboratory solvent, including, but not limited to, solvent systems comprising isopropanol and/or methyl tert-butyl ether. In certain embodiments, the amorphous form of the tri-HCl salt is prepared by a procedure comprising precipitation, spray drying, or lyophilization.

In certain embodiments, the amorphous form of the tri-HCl salt of Compound A1 comprises a specific quantity of solvent. For example, in certain embodiments, the tri-HCl comprises between about 0% and about 15% solvent (e.g., about 8% solvent) on a weight basis. In a particular embodiment, the tri-HCl salt comprises between about 0% and about 15% water (e.g., about 3% water) on a weight basis.

### Sulfate Salt of Compound A1

As used herein, a “sulfate salt” or “sulphate salt” of Compound A1 is a salt formed, e.g., by reacting Compound A1 with sulfuric acid. In certain embodiments, a sample of the sulfate salt of Compound A1 comprises about one mole of sulfate ion per mole of Compound A1 (e.g., an amount of sulfate ion per mole of Compound A1 equal to about 0.75, about 0.80, about 0.85, about 0.90, about 0.95, about 1.00, about 1.05, about 1.10, about 1.15, about 1.20, or about 1.25 molar equivalents of sulfate ion per mole of Compound A1). In certain embodiments, a sample of the sulfate salt of Compound A1 comprises about two moles of sulfate ion per mole of Compound A1 (e.g., an amount of sulfate ion per mole of Compound A1 equal to about 1.75, about 1.80, about 1.85, about 1.90, about 1.95, about 2.00, about 2.05, about 2.10, about 2.15, about 2.20, or about 2.25 molar equivalents of sulfate ion per mole of Compound A1).

### Amorphous Form of the Sulfate Salt of Compound A1

Certain embodiments herein provide an amorphous form of the sulfate salt of Compound A1.

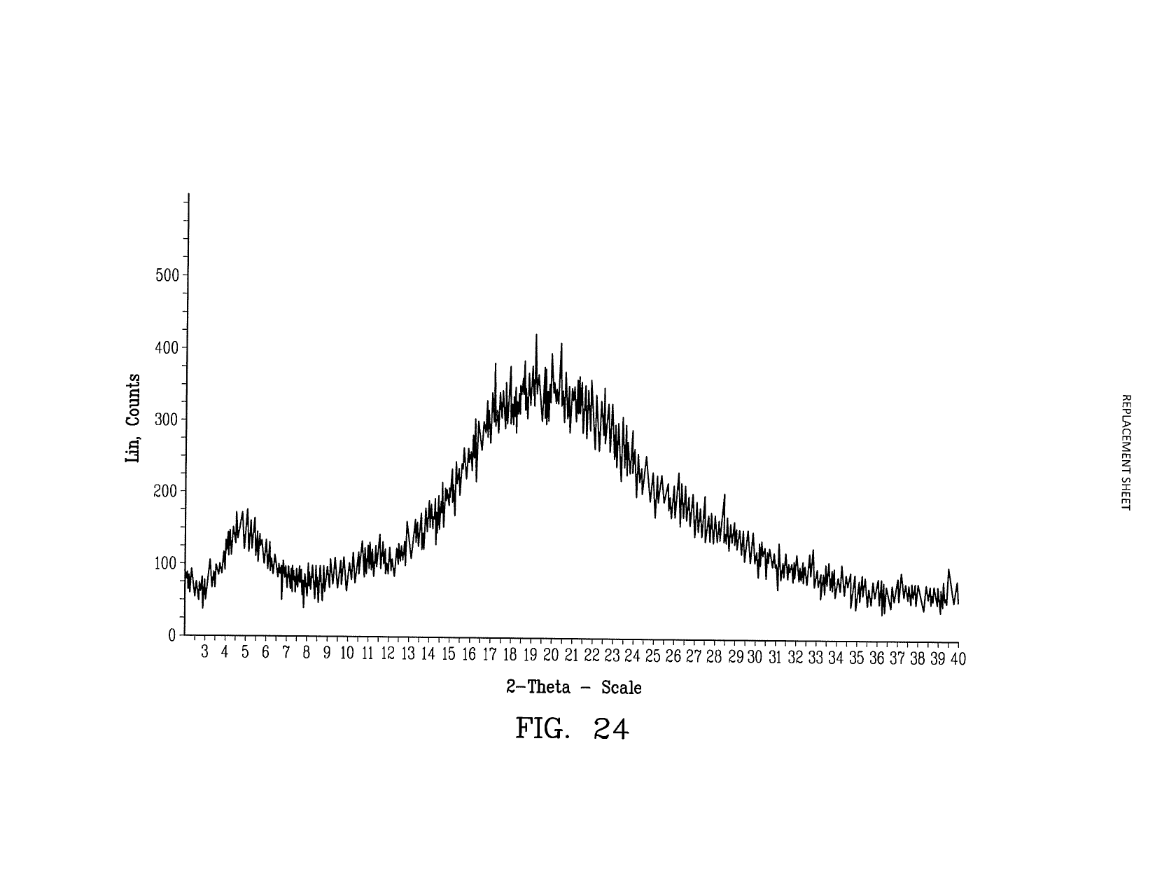

In certain embodiments, the amorphous form of the sulfate salt of Compound A1 exhibits particular structural characteristics, as determined, e.g., by diffraction analysis. A representative XRPD pattern of the amorphous form of the sulfate salt of Compound A1 is provided in FIG. 24. In certain embodiments, the amorphous form of the sulfate salt is characterized by an XRPD pattern with no peaks (reflections) indicative of crystal lattice planes and/or long range order. In certain embodiments, the amorphous form of the sulfate salt is characterized by an XRPD pattern with fewer than 10, fewer than 9, fewer than 8, fewer than 7, fewer than 6, fewer than 5, fewer than 4, fewer than 3, or fewer than 2 peaks (reflections) indicative of crystal lattice planes and/or long range order. In certain embodiments, the amorphous form of the sulfate salt of Compound A1 is characterized by an XRPD pattern which matches the pattern exhibited in FIG. 24.

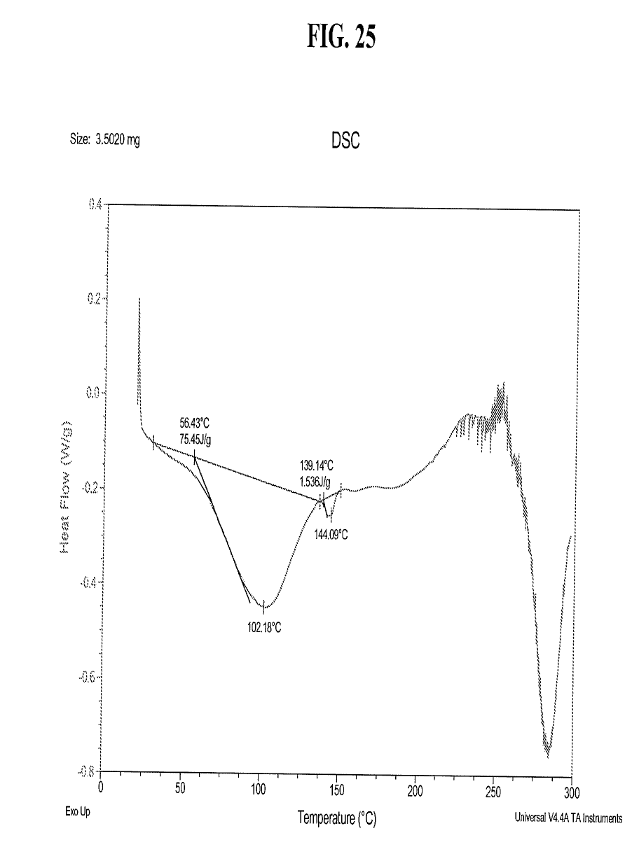

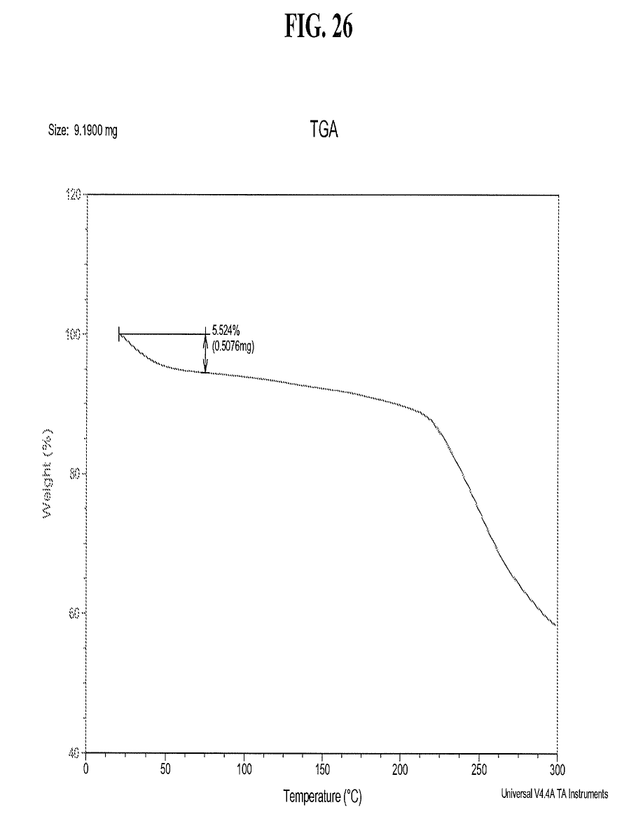

In certain embodiments, the amorphous form of the sulfate salt of Compound A1 exhibits particular thermal characteristics. Representative thermal characteristics of the amorphous form of the sulfate salt of Compound A1 are shown in FIG. 25 and FIG. 26. The representative DSC thermogram presented FIG. 25 comprises at least one endothermic event between about ambient temperature and about 150° C. The representative TGA thermogram presented in FIG. 26 comprises a mass loss between about 0% and about 20% (e.g., 5.5%) of the total mass of the sample upon heating from ambient temperature to about 75° C. In certain embodiments, the amorphous form of the sulfate salt exhibits a DSC thermogram which matches the representative DSC thermogram in FIG. 25. In certain embodiments, the amorphous form of the sulfate salt exhibits a TGA thermogram which matches the representative TGA thermogram in FIG. 26.

In certain embodiments, the amorphous form of the sulfate salt of Compound A1 exhibits characteristic chemical stability parameters. For example, in certain embodiments, a sample of the amorphous form of the sulfate salt exhibits a total chemical purity of about 93%, about 92%, about 91%, about 90%, about 89%, about 88%, about 87%, about 86%, or about 85% upon storage at about 40° C. and about 75% RH for 2 weeks and/or 4 weeks. In certain embodiments, a sample of the amorphous form of the sulfate salt exhibits a total chemical purity of about 83%, about 82%, about 81%, about 80%, about 79%, about 78%, or about 77% upon storage at about 60° C. and about 80% RH for 2 weeks and/or 4 weeks. In certain embodiments, a sample of the amorphous form of the sulfate salt exhibits a total chemical purity of about 90%, about 89%, about 88%, about 87%, about 86%, about 85%, about 84%, about 83%, or about 82% upon storage at about 80° C. for 2 weeks and/or 4 weeks.

In particular embodiments, a sample of the sulfate salt of Compound A1 is substantially amorphous. In certain embodiments, a sample of the amorphous form of the sulfate salt of Compound A1 contains birefringent particles comprising less than about 50%, less than about 40%, less than about 30%, less than about 20%, less than about 10%, less than about 5%, less than about 4%, less than about 3%, less than about 2%, or less than about 1% of the total number of particles in the sample.

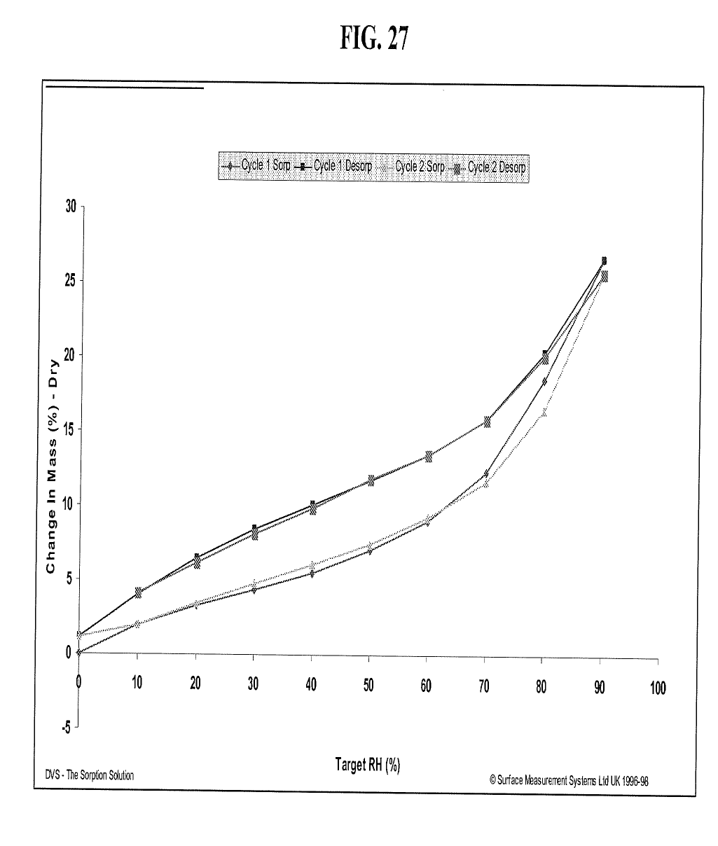

In certain embodiments, the amorphous form of the sulfate salt of Compound A1 exhibits particular characteristics with respect to moisture sorption. For example, in certain embodiments, the amorphous form of the sulfate salt of Compound A1 exhibits a moisture sorption profile matching the profile provided in FIG. 27. In certain embodiments, a sample of the amorphous form of the sulfate salt of Compound A1 gains less than about 30%, less than about 25%, less than about 20%, less than about 15%, or less than about 10% weight when increased from about 0% RH to about 90% RH at about ambient temperature. In certain embodiments, the weight gain is reversible upon decreasing from about 90% RH to about 0% RH at about ambient temperature. In certain embodiments, the amorphous form of the sulfate salt is substantially physically stable when subjected to the moisture sorption/desorption program (e.g., does not exhibit deliquescence, does not undergo crystallization, and/or does not exhibit morphological change as observed by polarized light microscopy).

In certain embodiments, the amorphous form of the sulfate salt of Compound A1 can be obtained from any suitable laboratory solvent(s), including, but not limited to, solvent systems comprising ethyl acetate, isopropyl acetate, and/or isopropanol. In certain embodiments, the amorphous form of the sulfate salt is prepared by a procedure comprising precipitation, spray drying, or lyophilization.

In certain embodiments, the amorphous form of the sulfate salt of Compound A1 comprises a specific quantity of solvent. For example, in certain embodiments, the sulfate salt comprises between about 0% and about 15% solvent (e.g., about 5.5% solvent) on a weight basis.

### Mesylate Salt of Compound A1

Particular salts described herein include “mesylate salts” or “methanesulfonic acid salts” of Compound A1. A mesylate salt of Compound A1 is an acid addition salt formed, e.g., by reacting Compound A1 with methanesulfonic acid. Particular mesylate salts of Compound A1 provided herein comprise approximately 1 molar equivalent of methanesulfonic acid ion per mole of Compound A1. In specific embodiments, a mesylate salt of Compound A1 comprises about 0.75, about 0.80, about 0.85, about 0.90, about 0.95, about 1.00, about 1.05, about 1.10, about 1.15, about 1.20, or about 1.25 molar equivalents of methanesulfonate ion per mole of Compound A1. Particular mesylate salts of Compound A1 provided herein comprise approximately 2 molar equivalent of methanesulfonic acid ion per mole of Compound A1. In specific embodiments, a mesylate salt of Compound A1 comprises about 1.75, about 1.80, about 1.85, about 1.90, about 1.95, about 2.00, about 2.05, about 2.10, about 2.15, about 2.20, or about 2.25 molar equivalents of methanesulfonate ion per mole of Compound A1.

### Form I of the Mesylate Salt of Compound A1

Certain embodiments herein provide Form I of the mesylate salt of Compound A1. In some embodiments, the Form I of the mesylate salt of Compound A1 is isolated.

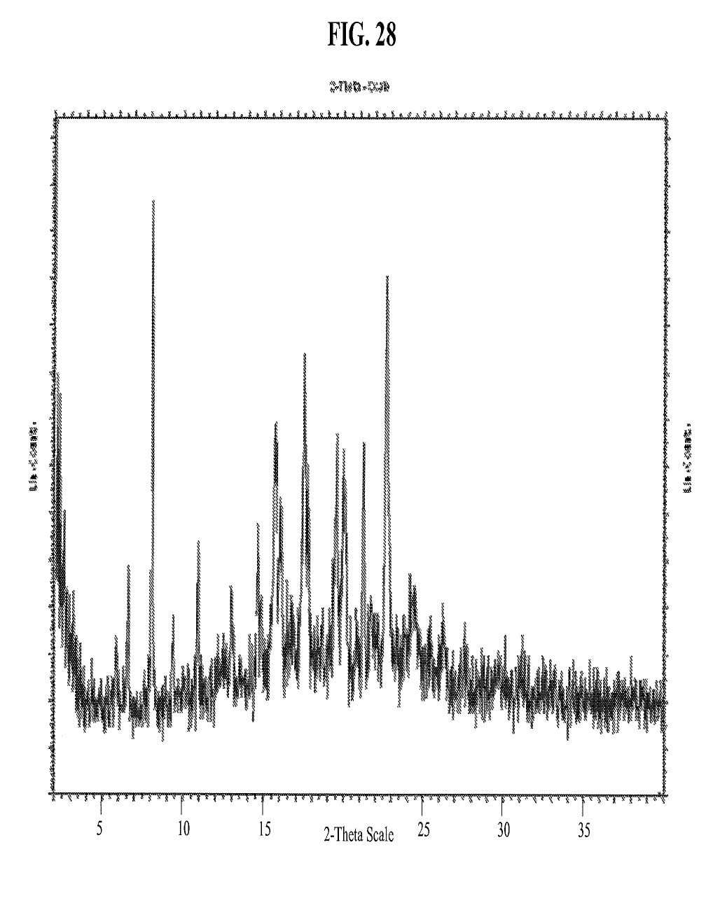

In certain embodiments, Form I of the mesylate salt of Compound A1 exhibits particular structural characteristics, as determined, e.g., by diffraction analysis. A representative XRPD pattern of Form I of the mesylate salt of Compound A1 is provided in FIG. 28. In certain embodiments, Form I of the mesylate salt of Compound A1 is characterized by XRPD peaks located at any one, two, three, four, five, six, seven, eight, nine, ten, eleven, twelve, or thirteen of the following approximate positions: 5.8, 6.5, 8.0, 9.4, 10.7, 13.0, 14.6, 15.5, 17.5, 19.5, 20.5, 21.6, and 22.9 degrees 2θ. In some embodiments, Form I of the mesylate salt of Compound A1 is characterized by at least 8, at least 9, or at least 10 of said approximate positions. In certain embodiments, Form I of the mesylate salt of Compound A1 is characterized by XRPD peaks located at about 8.0, 17.5, and 22.9 degrees 2θ. In certain embodiments, Form I of the mesylate salt of Compound A1 is characterized by XRPD peaks located at about 15.5, 19.5, 20.5 degrees 2θ. In certain embodiments, Form I of the mesylate salt of Compound A1 is characterized by XRPD peaks located at about 6.5, 10.7, and 21.6 degrees 2θ. In certain embodiments, Form I of the mesylate salt of Compound A1 is characterized by XRPD peaks at about 6.5, 8.0, 10.7, 15.5, 17.5, 19.5, 20.5, 21.6, and 22.9 degrees 2θ. In certain embodiments, Form I of the mesylate salt of Compound A1 is characterized by XRPD peaks at about 6.5, 8.0, 10.7, 13.0, 15.5, 17.5, 19.5, 20.5, 21.6, and 22.9 degrees 2θ. In certain embodiments, Form I of the mesylate salt of Compound A1 is characterized by an XRPD pattern which matches the pattern exhibited in FIG. 28. In some embodiments, provided herein is an isolated Form I mesylate salt of Compound A1, which has an XRPD pattern which matches the pattern of FIG. 28. In some embodiments, provided herein is an isolated Form I mesylate salt of Compound A1, which has an XRPD pattern comprising peaks at about 8.0, 17.5, and 22.9 degrees 2θ. In particular embodiments, a sample of the mesylate salt of Compound A1 is substantially crystalline.

In certain embodiments, a sample of Form I of the mesylate salt of Compound A1 contains birefringent particles comprising about 100%, about 90%, about 80%, about 70%, about 60%, or about 50% of the total number of particles in the sample.

In certain embodiments, Form I of the mesylate salt of Compound A1 is chemically pure. In certain embodiments, Form I of the mesylate salt of Compound A1 is physically pure.

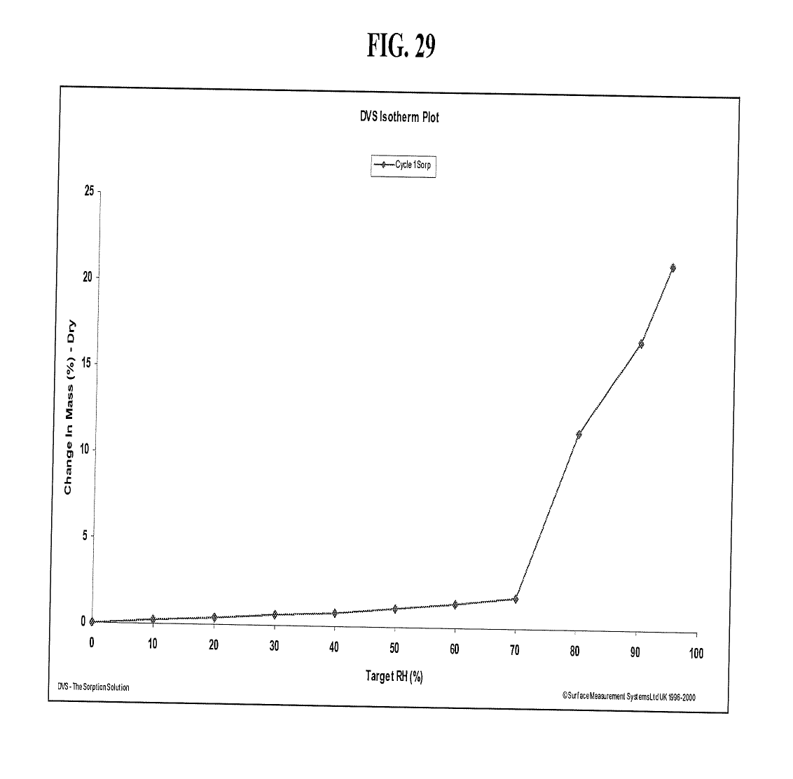

In certain embodiments, Form I of the mesylate salt of Compound A1 exhibits particular characteristics with respect to moisture sorption. For example, in certain embodiments, Form I of the mesylate salt of Compound A1 exhibits a moisture sorption profile matching the profile provided in FIG. 29. In certain embodiments, a sample of Form I of the mesylate salt of Compound A1 gains less than about 10%, less than about 9%, less than about 8%, less than about 7%, less than about 6%, less than about 5%, less than about 4%, less than about 3%, less than about 2%, or less than about 1% weight when increased from about 0% RH to about 70% RH at about ambient temperature. In certain embodiments, a sample of Form I of the mesylate salt of Compound A1 gains less than about 30%, less than about 25%, less than about 20%, less than about 15%, or less than about 10% weight when increased from about 0% RH to about 95% RH at about ambient temperature.

In certain embodiments, Form I of the mesylate salt can be obtained from any suitable laboratory solvent, including, but not limited to, solvent systems comprising ethyl acetate, acetonitrile, water, dichloromethane, petroleum ether, ethanol, toluene, isopropyl acetate, isopropanol, acetone, or a mixture of two or more thereof. In certain embodiments, the solvent system comprises a common laboratory solvent, as known in the art. In certain embodiments, Form I of the mesylate salt of Compound A1 may be obtained by performing any three, four, five, six, or seven of the following steps: (a) obtain a first solution comprising the free base of Compound A1; (b) obtain a second solution comprising methanesulfonic acid; (c) heat the first solution to a temperature above ambient temperature; (d) admix the first solution and the second solution such that the resulting mixture comprises approximately one or approximately two molar equivalents of methanesulfonic acid per mole of Compound A1; (e) cool the mixture to a temperature approximately equal to or below ambient temperature; (f) isolate Form I of the mesylate salt of Compound A1; and (g) dry Form I of the mesylate salt.

In certain embodiments, the first solution in step (a) comprises a common laboratory solvent, as described herein an/or as known in the art. In certain embodiments, the second solution in step (b) comprises a common laboratory solvent, as described herein an/or as known in the art. In certain embodiments, the temperature in step (c) is above about 25° C., above about 30° C., above about 40° C., above about 50° C., above about 60° C., or above about 70° C. In certain embodiments, the temperature in step (c) is about 35° C., about 40° C., about 45° C., about 50° C., about 55° C., about 60° C., about 65° C., about 70° C., or about 75° C. In certain embodiments, the resulting mixture in step (d) comprises about 0.75, about 0.80, about 0.85, about 0.90, about 0.95, about 1.00, about 1.05, about 1.10, about 1.15, about 1.20, about 1.25, about 1.30, about 1.35, about 1.40, about 1.45, about 1.50, about 1.55, about 1.60, about 1.65, about 1.70, about 1.75, about 1.80, about 1.85, about 1.90, about 1.95, about 2.00, about 2.05, about 2.10, about 2.15, about 2.20, or about 2.25 molar equivalents of methanesulfonic acid per mole of Compound A1. In certain embodiments, the temperature in step (e) is about 25° C., about 20° C., about 15° C., about 10° C., about 5° C., about 0° C., or less than 0° C. In certain embodiments, the isolation in step (f) is comprises suction filtration. In certain embodiments, the drying in step (g) comprises vacuum drying. In certain embodiments, the drying in step (g) comprises drying at or below about 70° C., at or below about 60° C., at or below about 50° C., at or about 40° C., at below about 30° C., or at about ambient temperature.

In particular embodiments, Form I of the mesylate salt of Compound A1 showed advantageous properties including properties relating to, e.g., crystallinity and water uptake (e.g., hygroscopicity).

### Phosphate Salt of Compound A1

As used herein, a “phosphate salt” of Compound A1 is a salt formed, e.g., by reacting Compound A1 and phosphoric acid. In certain embodiments, a sample of the phosphate salt of Compound A1 comprises an amount of phosphate ion per mole of Compound A1 equal to about 0.75, about 0.80, about 0.85, about 0.90, about 0.95, about 1.00, about 1.05, about 1.10, about 1.15, about 1.20, or about 1.25 molar equivalents of phosphate ion per mole of Compound A1.

### Amorphous Form of the Phosphate Salt of Compound A1

Certain embodiments herein provide an amorphous form of the phosphate salt of Compound A1.

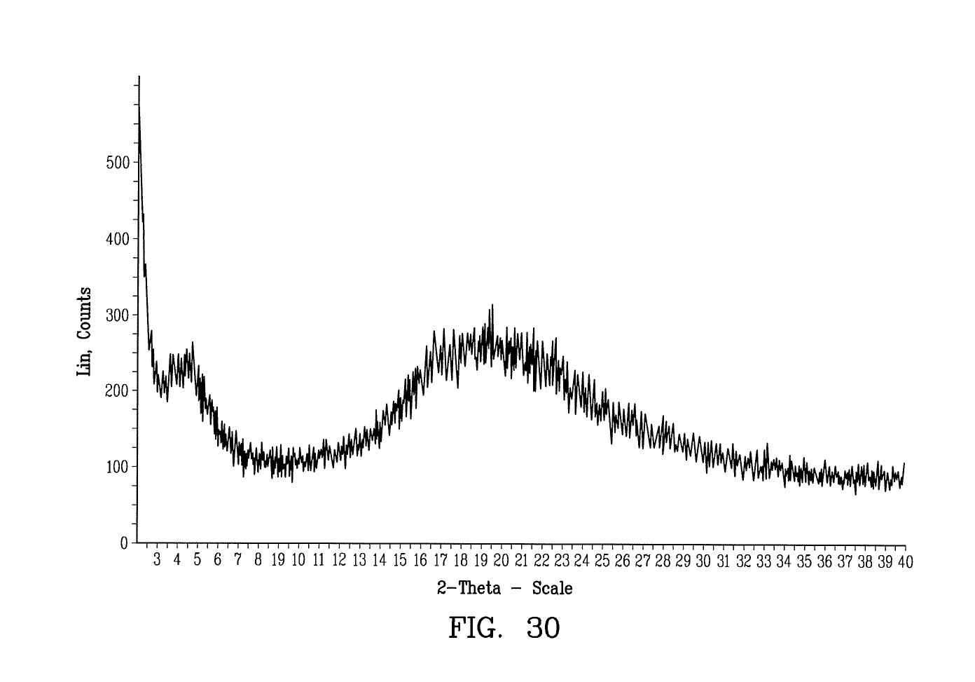

In certain embodiments, the amorphous form of the phosphate salt of Compound A1 exhibits particular structural characteristics, as determined, e.g., by diffraction analysis. A representative XRPD pattern of the amorphous form of the phosphate salt of Compound A1 is provided in FIG. 30. In certain embodiments, the amorphous form of the phosphate salt is characterized by an XRPD pattern with no peaks (reflections) indicative of crystal lattice planes and/or long range order. In certain embodiments, the amorphous form of the phosphate salt is characterized by an XRPD pattern with fewer than 10, fewer than 9, fewer than 8, fewer than 7, fewer than 6, fewer than 5, fewer than 4, fewer than 3, or fewer than 2 peaks (reflections) indicative of crystal lattice planes and/or long range order. In certain embodiments, the amorphous form of the phosphate salt of Compound A1 is characterized by an XRPD pattern which matches the pattern exhibited in FIG. 30.

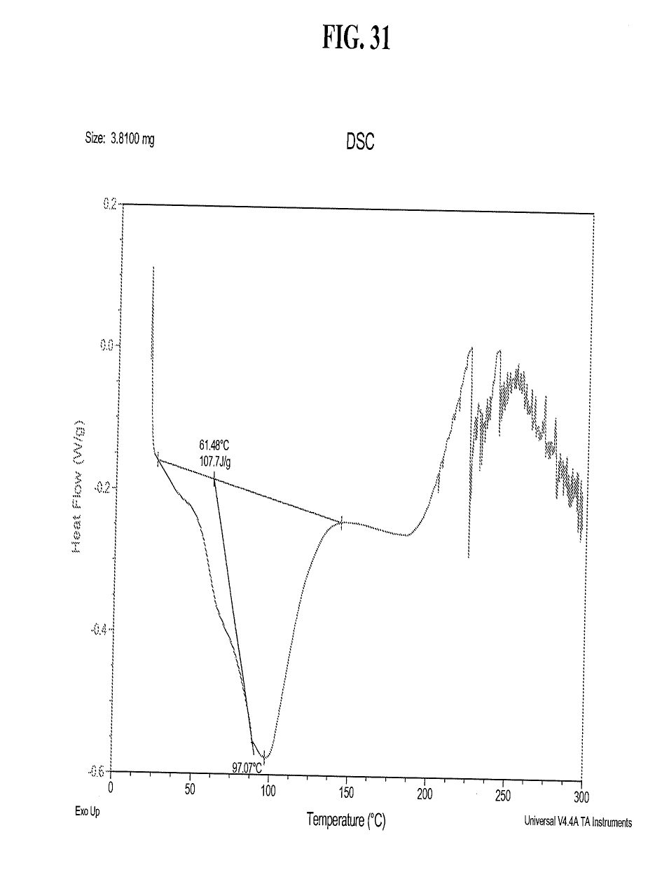

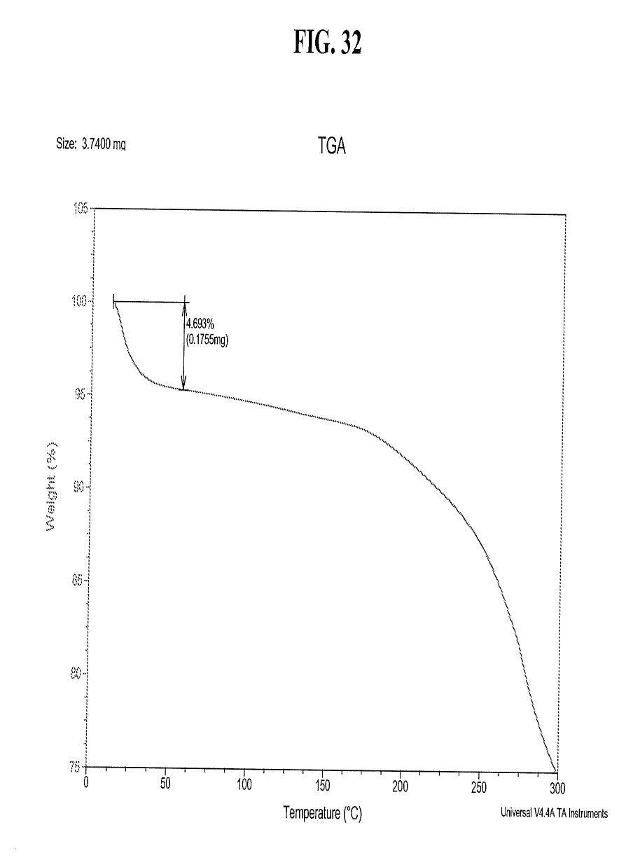

In certain embodiments, the amorphous form of the phosphate salt of Compound A1 exhibits particular thermal characteristics. Representative thermal characteristics of the amorphous form of the phosphate salt of Compound A1 are shown in FIG. 31 and FIG. 32. The representative DSC thermogram presented FIG. 31 comprises at least one endothermic event between about ambient temperature and about 150° C. The representative TGA thermogram presented in FIG. 32 comprises a mass loss between about 0% and about 20% (e.g., 4.7%) of the total mass of the sample upon heating from ambient temperature to about 60° C. In certain embodiments, the amorphous form of the phosphate salt exhibits a DSC thermogram which matches the representative DSC thermogram in FIG. 31. In certain embodiments, the amorphous form of the phosphate salt exhibits a TGA thermogram which matches the representative TGA thermogram in FIG. 32.

In certain embodiments, the amorphous form of the phosphate salt of Compound A1 exhibits characteristic chemical stability parameters. For example, in certain embodiments, a sample of the amorphous form of the phosphate salt exhibits a total chemical purity of about 93%, about 92%, about 91%, about 90%, about 89%, about 88%, about 87%, about 86%, or about 85% upon storage at about 40° C. and about 75% RH for 2 weeks and/or 4 weeks. In certain embodiments, a sample of the amorphous form of the phosphate salt exhibits a total chemical purity of about 83%, about 82%, about 81%, about 80%, about 79%, about 78%, about 77%, about 76%, about 75%, about 74%, about 73% or about 72% upon storage at about 60° C. and about 80% RH for 2 weeks and/or 4 weeks. In certain embodiments, a sample of the amorphous form of the phosphate salt exhibits a total chemical purity of about 88%, about 87%, about 86%, about 85%, about 84%, about 83%, or about 82% upon storage at about 80° C. for 2 weeks and/or 4 weeks.

In particular embodiments, a sample of the phosphate salt of Compound A1 is substantially amorphous. In certain embodiments, a sample of the amorphous form of the phosphate salt of Compound A1 contains birefringent particles comprising less than about 50%, less than about 40%, less than about 30%, less than about 20%, less than about 10%, less than about 5%, less than about 4%, less than about 3%, less than about 2%, or less than about 1% of the total number of particles in the sample.

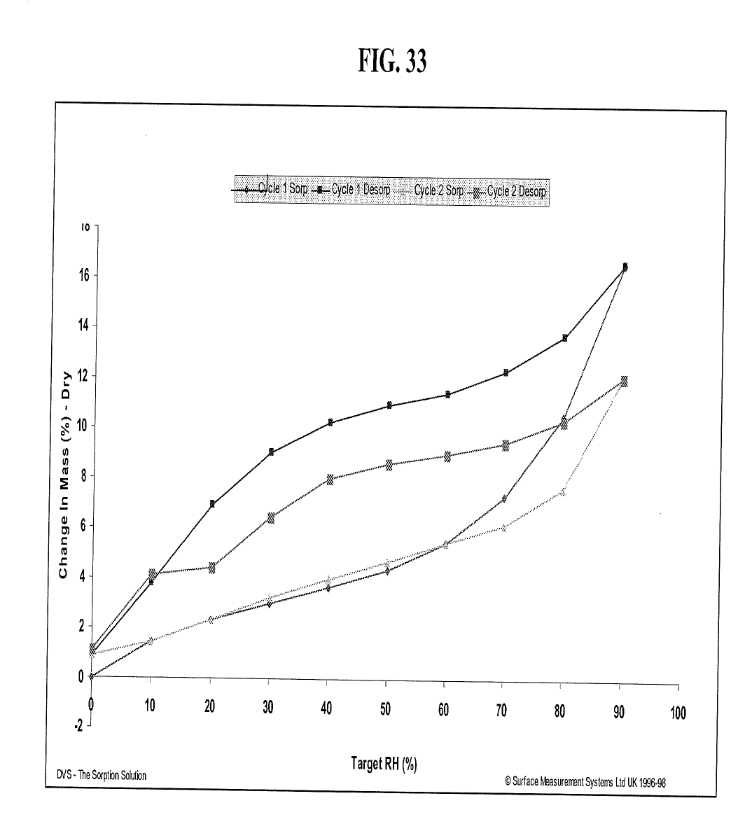

In certain embodiments, the amorphous form of the phosphate salt of Compound A1 exhibits particular characteristics with respect to moisture sorption. For example, in certain embodiments, the amorphous form of the phosphate salt of Compound A1 exhibits a moisture sorption profile matching the profile provided in FIG. 33. In certain embodiments, a sample of the amorphous form of the phosphate salt of Compound A1 gains less than about 20%, less than about 15%, less than about 10%, less than about 5%, or about 0% weight when increased from about 0% RH to about 90% RH at about ambient temperature. In certain embodiments, the weight gain is reversible upon decreasing from about 90% RH to about 0% RH at about ambient temperature. In certain embodiments, the amorphous form of the phosphate salt is substantially physically stable when subjected to the moisture sorption/desorption program (e.g., does not exhibit deliquescence, does not undergo crystallization as observed by XRPD analysis, and/or does not exhibit morphological change as observed by polarized light microscopy).

In certain embodiments, the amorphous form of the phosphate salt of Compound A1 can be obtained from any suitable laboratory solvent(s), including, but not limited to, solvent systems comprising cyclopentylmethyl ether, ethanol, and/or ethyl acetate. In certain embodiments, the amorphous form of the phosphate salt is prepared by a procedure comprising precipitation, spray drying, or lyophilization.

In certain embodiments, the amorphous form of the phosphate salt of Compound A1 comprises a specific quantity of solvent. For example, in certain embodiments, the phosphate salt comprises between about 0% and about 15% solvent (e.g., about 4.7% solvent) on a weight basis.

### HBr Salt of Compound A1

As used herein, a “hydrobromide salt,” “HBr salt,” or “bromide salt” of Compound A1 is a salt formed, e.g., by reacting Compound A1 with hydrobromic acid. In certain embodiments, a sample of the HBr salt of Compound A1 comprises an amount of bromide ion per mole of Compound A1 equal to about 0.75, about 0.80, about 0.85, about 0.90, about 0.95, about 1.00, about 1.05, about 1.10, about 1.15, about 1.20, or about 1.25 molar equivalents of bromide ion per mole of Compound A1.

### Form I of the HBr Salt of Compound A1

Certain embodiments herein provide Form I of the HBr salt of Compound A1. In some embodiments, the Form I of the HBr salt of Compound A1 is isolated.

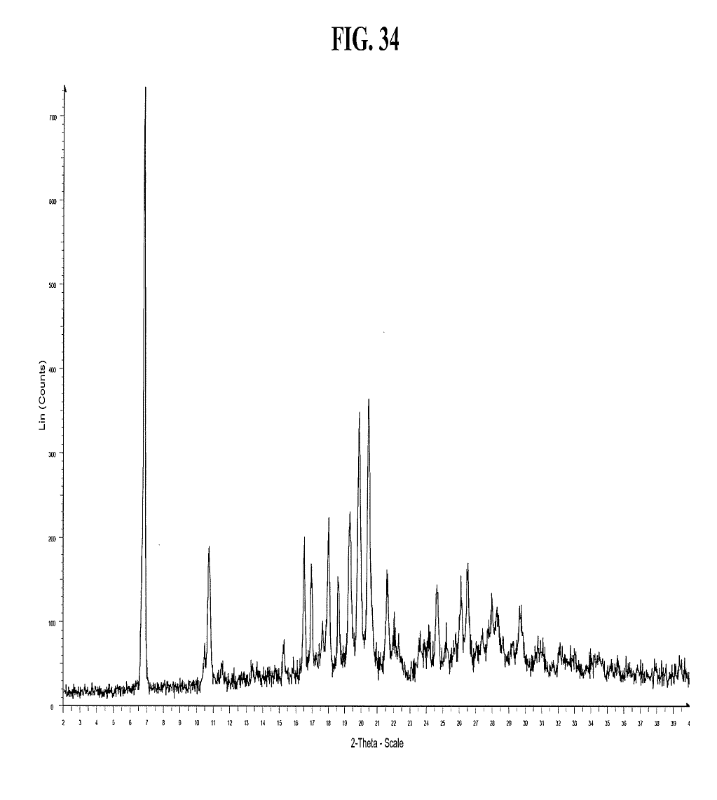

In certain embodiments, Form I of the HBr salt of Compound A1 exhibits particular structural characteristics, as determined, e.g., by diffraction analysis. A representative XRPD pattern of Form I of the HBr salt of Compound A1 is provided in FIG. 34. In certain embodiments, Form I of the HBr salt of Compound A1 is characterized by XRPD peaks located at any one, two, three, four, five, six, seven, eight, nine, ten, or eleven of the following approximate positions: 6.8, 10.6, 16.4, 16.9, 17.6, 17.8, 18.6, 19.4, 20.0, 20.5, and 21.6 degrees 2θ. In some embodiments, Form I of the HBr salt of Compound A1 is characterized by at least 8, at least 9, or at least 10 of said approximate positions. In certain embodiments, Form I of the HBr salt of Compound A1 is characterized by XRPD peaks located at about 6.8, 20.0, and 20.5 degrees 2θ. In certain embodiments, Form I of the HBr salt of Compound A1 is characterized by XRPD peaks located at about 10.6, 17.8, and 19.4 degrees 2θ. In certain embodiments, Form I of the HBr salt of Compound A1 is characterized by XRPD peaks located at about 16.4, 16.9, and 21.6 degrees 2θ. In some embodiments, Form I of the HBr salt of Compound A1 is characterized by XRPD peaks at about 6.8, 10.6, 16.4, 16.9, 17.8, 19.4, 20.0, 20.5, and 21.6 degrees 2θ. In certain embodiments, Form I of the HBr salt of Compound A1 is characterized by an XRPD pattern which matches the pattern exhibited in FIG. 34. In some embodiments, provided herein is an isolated Form I HBr salt of Compound A1, which has an XRPD pattern which matches FIG. 34. In some embodiments, provided herein is an isolated Form I HBr salt of Compound A1, which has an XRPD pattern comprising peaks at about 6.8, 20.0, and 20.5 degrees 2θ. In certain embodiments, a sample of Form I of the HBr salt of Compound A1 is substantially crystalline.

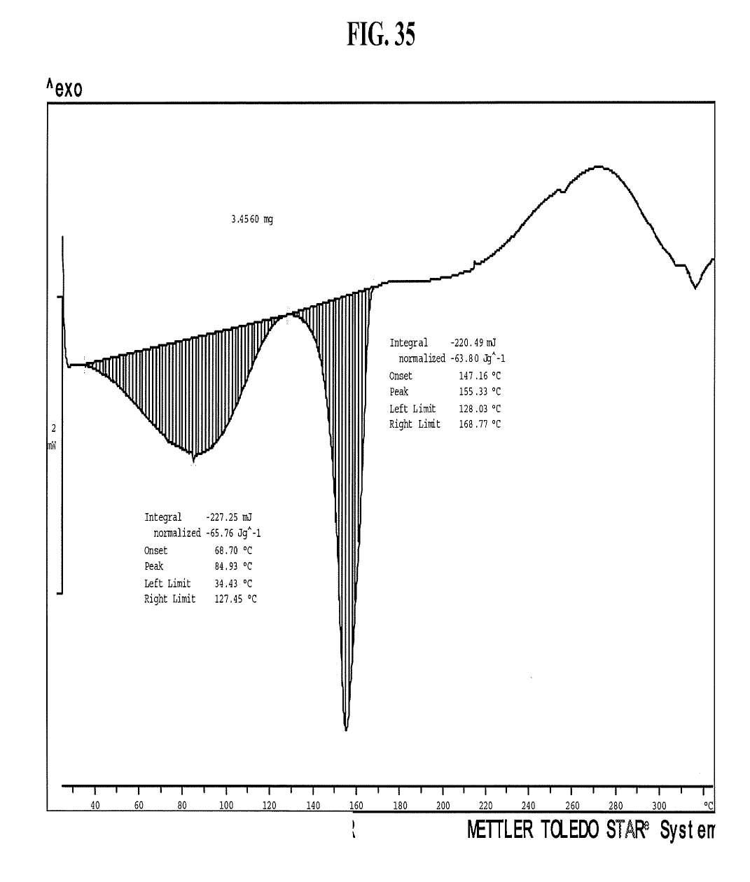

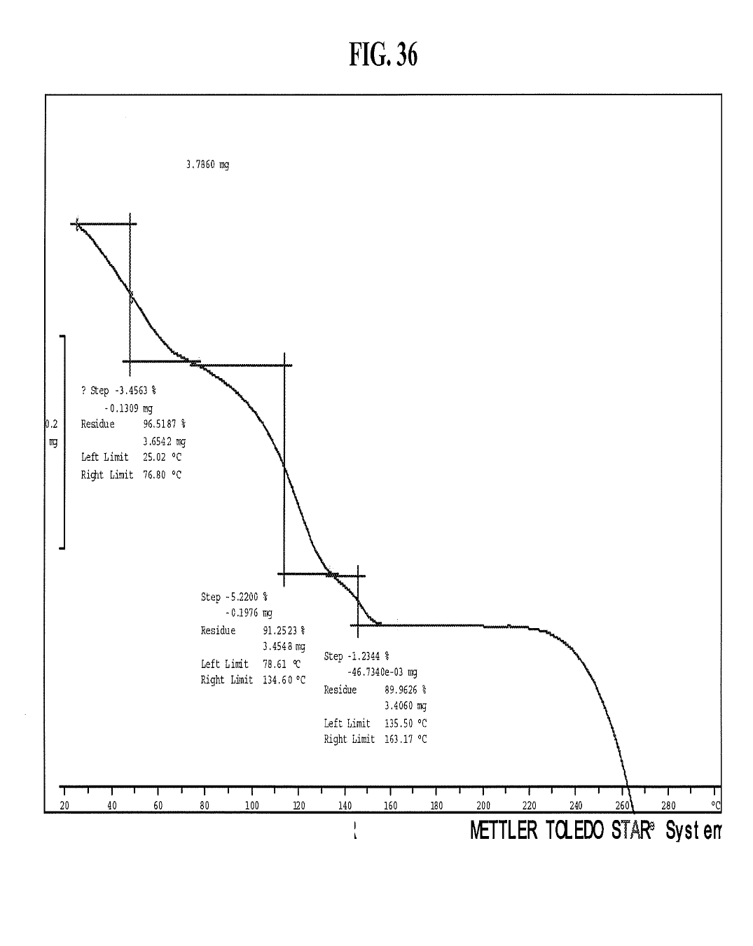

In certain embodiments, Form I of the HBr salt of Compound A1 exhibits particular thermal characteristics. Representative thermal characteristics of Form I of the HBr salt of Compound A1 are shown in FIG. 35 and FIG. 36. The representative DSC thermogram presented in FIG. 35 comprises at least one endothermic event between about ambient temperature and about 130° C., and at least one endothermic event with an onset temperature between about 130° C. and about 180° C. In certain embodiments, Form I of the HBr salt of Compound A1 exhibits a DSC thermogram that matches the DSC thermogram shown in FIG. 35. The representative TGA thermogram presented in FIG. 36 comprises (1) a mass loss of between about 0% and about 10% (e.g., about 3.5%) of the total mass of the sample upon heating from about ambient temperature to about 75° C., and (2) a mass loss of between about 0% and about 10% (e.g., about 5.2%) of the total mass of the sample upon heating from about 75° C. to about 135° C. In certain embodiments, the mass loss(es) correspond to a loss of solvent. In certain embodiments, Form I of the HBr salt of Compound A1 exhibits a TGA thermogram that matches the TGA thermogram shown in FIG. 36.

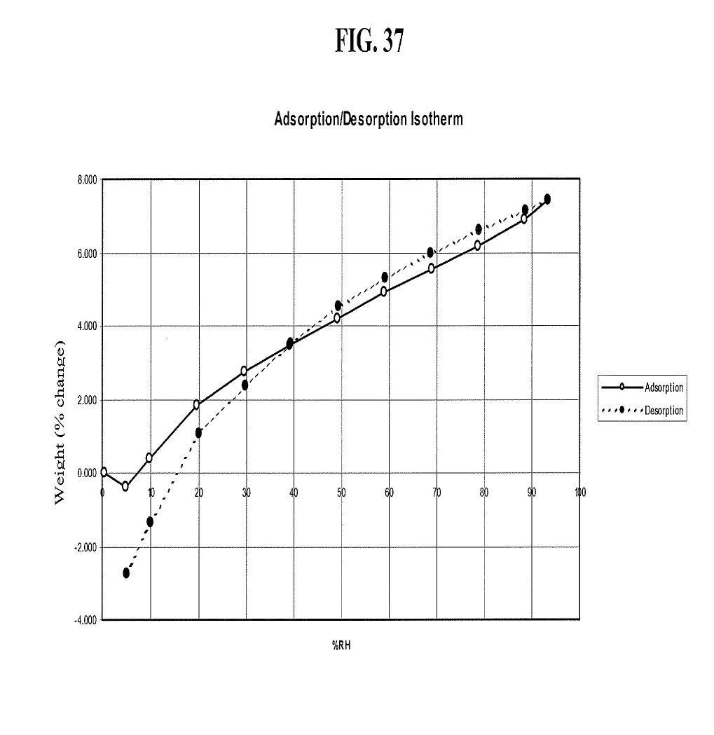

In certain embodiments, Form I of the HBr salt of Compound A1 exhibits particular characteristics with respect to moisture sorption. For example, in certain embodiments, Form I of the HBr salt of Compound A1 exhibits a moisture sorption profile matching the profile provided in FIG. 37. In certain embodiments, a sample of Form I of the HBr salt of Compound A1 gains less than about 20%, less than about 19%, less than about 18%, less than about 17%, less than about 16%, less than about 15%, less than about 14%, less than about 13%, less than about 12%, less than about 11%, less than about 10%, less than about 9%, less than about 8%, less than about 7%, less than about 6%, or less than about 5% weight when increased from about 0% RH to about 90% RH at about ambient temperature. In certain embodiments, the weight gain is reversible upon decreasing from about 90% RH to about 0% RH at about ambient temperature.

In certain embodiments, Form I of the HBr salt can be obtained from any suitable laboratory solvent, including, but not limited to, solvent systems comprising ethyl acetate, acetonitrile, water, dichloromethane, petroleum ether, ethanol, toluene, isopropyl acetate, isopropanol, acetone, or a mixture of two or more thereof. In certain embodiments, the solvent system comprises a common laboratory solvent, as known in the art.

### Sesquifumarate Salt of Compound A1

Particular salts described herein include “fumarate salts” of Compound A1. A fumarate salt of Compound A1 is an acid addition salt formed, e.g., by reacting Compound A1 with fumaric acid.

A “sesquifumarate salt” of Compound A1 is a salt which comprises approximately 1.5 molar equivalents of fumarate ion per mole of Compound A1. In specific embodiments, a sesquifumarate salt of Compound A1 comprises between about 1.25 and about 1.75 molar equivalents of fumarate ion per mole of Compound A1. In specific embodiments, a sesquifumarate salt of Compound A1 comprises about 1.25, about 1.30, about 1.35, about 1.40, about 1.45, about 1.50, about 1.55, about 1.60, about 1.65, about 1.70, or about 1.75 molar equivalents of fumarate ion per mole of Compound A1.

### Form I of the Sesquifumarate Salt of Compound A1

Certain embodiments herein provide Form I of the sesquifumarate salt of Compound A1. In some embodiments, the Form I of the sesquifumarate salt of Compound A1 is isolated.

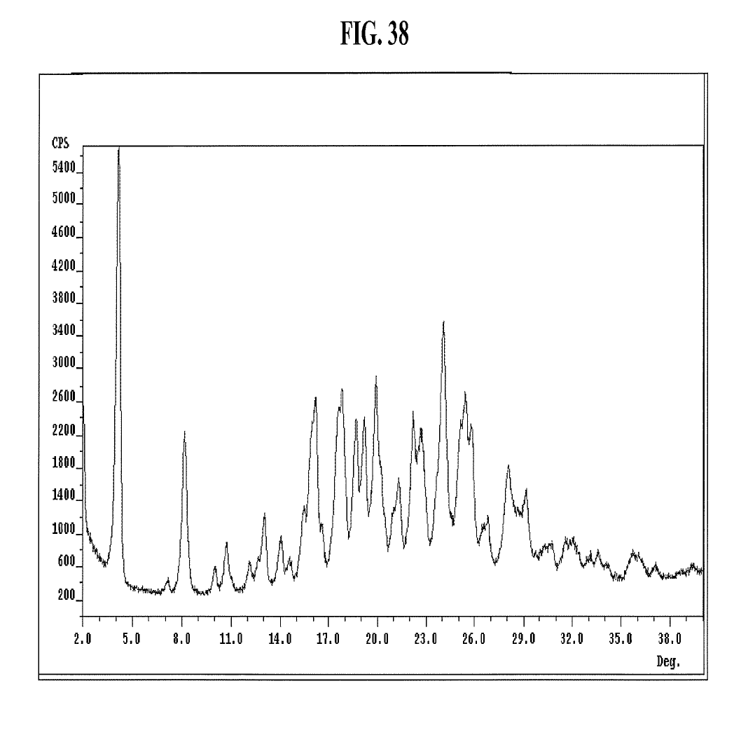

In certain embodiments, Form I of the sesquifumarate salt of Compound A1 exhibits particular structural characteristics, as determined, e.g., by diffraction analysis. A representative XRPD pattern of Form I of the sesquifumarate salt of Compound A1 is provided in FIG. 38. In certain embodiments, Form I of the sesquifumarate salt of Compound A1 is characterized by XRPD peaks located at any one, two, three, four, five, six, seven, eight, nine, ten, eleven, twelve, thirteen, or fourteen of the following approximate positions: 4.0, 8.0, 10.7, 13.0, 14.0, 16.0, 17.9, 18.8, 19.2, 19.9, 22.2, 22.7, 24.1, and 25.4 degrees 2θ. In some embodiments, Form I of the sesquifumarate salt of Compound A1 is characterized by at least 8, at least 9, or at least 10 of said approximate positions. In certain embodiments, Form I of the sesquifumarate salt of Compound A1 is characterized by XRPD peaks located at about 4.0, 8.0, and 24.1 degrees 2θ. In certain embodiments, Form I of the sesquifumarate salt of Compound A1 is characterized by XRPD peaks located at about 16.0, 17.9, and 19.9 degrees 2θ. In certain embodiments, Form I of the sesquifumarate salt of Compound A1 is characterized by XRPD peaks located at about 18.8, 19.2, and 25.4 degrees 2θ. In some embodiments, Form I of the sesquifumarate salt of Compound A1 is characterized by XRPD peaks at about 4.0, 8.0, 16.0, 17.9, 18.8, 19.2, 19.9, 24.1, and 25.4 degrees 2θ. In some embodiments, Form I of the sesquifumarate salt of Compound A1 is characterized by XRPD peaks at about 4.0, 8.0, 16.0, 17.9, 18.8, 19.2, 19.9, 22.2, 24.1, and 25.4 degrees 2θ. In certain embodiments, Form I of the sesquifumarate salt of Compound A1 is characterized by an XRPD pattern which matches the pattern exhibited in FIG. 38. In some embodiments, provided herein is an isolated Form I sesquifumarate salt of Compound A1, which has an XRPD pattern which matches the pattern exhibited in FIG. 38. In some embodiments, provided herein is an isolated Form I sesquifumarate salt of Compound A1, which has an XRPD pattern comprising peaks at about 4.0, 8.0, and 24.1 degrees 2θ. In particular embodiments, a sample of the sesquifumarate salt of Compound A1 is substantially crystalline.

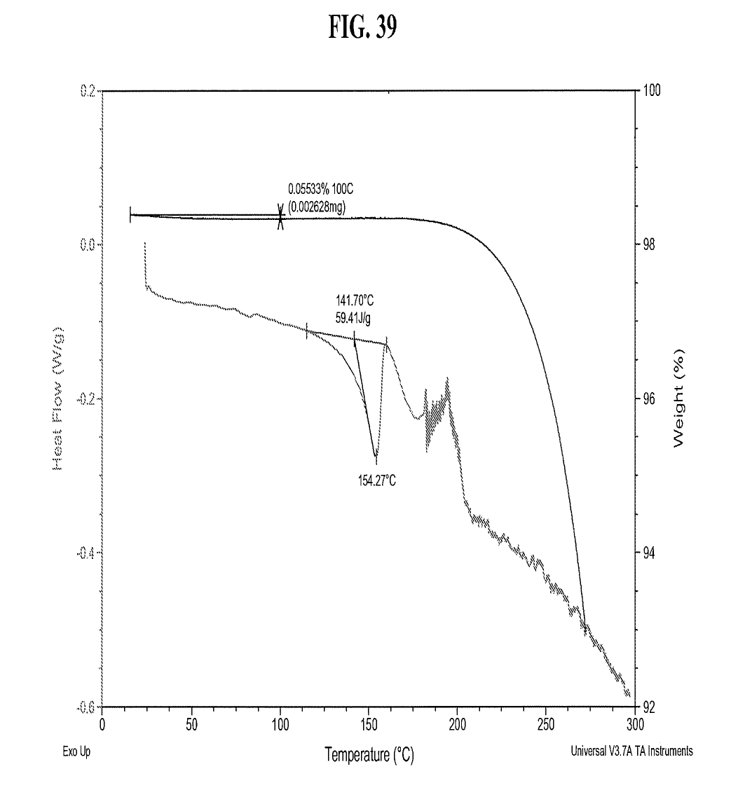

In certain embodiments, Form I of the sesquifumarate salt of Compound A1 exhibits particular thermal characteristics. Representative thermal characteristics of Form I of the sesquifumarate salt of Compound A1 are shown in FIG. 39. The representative DSC thermogram presented in FIG. 39 comprises an endothermic event with an onset temperature of about 142° C. and a peak temperature of about 154° C. In certain embodiments, Form I of the sesquifumarate salt of Compound A1 exhibits a DSC thermogram comprising an endothermic event between about 100° C. and about 160° C. In certain embodiments, Form I of the sesquifumarate salt of Compound A1 exhibits a DSC thermogram comprising an endothermic event with an onset temperature and/or peak temperature at about 125° C., 126° C., 127° C., 128° C., 129° C., 130° C., 131° C., 132° C., 133° C., 134° C., 135° C., 136° C., 137° C., 138° C., 139° C., 140° C., 141° C., 142° C., 143° C., 144° C., 145° C., 146° C., 147° C., 148° C., 149° C., 150° C., 151° C., 152° C., 153° C., 154° C., 155° C., 156° C., 157° C., 158° C., 159° C., or 160° C. In certain embodiments, Form I of the sesquifumarate salt exhibits a DSC thermogram matching the DSC thermogram displayed in FIG. 39.

The representative TGA thermogram presented in FIG. 39 comprises a mass loss of about 0% of the total mass of the sample upon heating from about ambient temperature to about 100° C. In certain embodiments, Form I of the sesquifumarate salt of Compound A1 exhibits a TGA thermogram comprising a mass loss of between about 0% and about 10% of the total mass of the sample. In certain embodiments, Form I of the sesquifumarate salt of Compound A1 exhibits a TGA thermogram comprising a mass loss of about 0%, about 1%, about 2%, about 3%, about 4%, about 5%, about 6%, about 7%, about 8%, about 9%, or about 10% of the total mass of the sample when heated from about ambient temperature to about 100° C. In certain embodiments, an observed mass loss corresponds to a loss of solvent (such as, e.g., water and/or an alcohol). In certain embodiments, Form I of the sesquifumarate salt exhibits a TGA thermogram matching the TGA thermogram displayed in FIG. 39.

In certain embodiments, Form I of the sesquifumarate salt of Compound A1 exhibits characteristic chemical stability parameters. For example, in certain embodiments, a sample of Form I of the sesquifumarate salt exhibits a chemical purity about 75%, about 76%, about 77%, about 78%, about 79%, about 79%, about 80%, about 81%, about 82%, about 83%, about 84%, about 85%, about 86%, about 87%, about 88%, about 89%, or about 90% upon storage at about 60° C. and about 80% RH after 2 weeks and/or after 4 weeks. In certain embodiments, a sample of Form I of the sesquifumarate salt exhibits a chemical purity of about 80%, about 81%, about 82%, about 83%, about 84%, about 85%, about 86%, about 87%, about 88%, about 89%, about 90%, about 91%, about 92%, about 93%, about 94%, or about 95% upon storage at about 80° C. for 2 weeks and/or 4 weeks. In certain embodiments, a sample of Form I of the sesquifumarate salt exhibits a chemical purity of about 90%, about 91%, about 92%, about 93%, about 94%, about 95%, about 96%, about 97%, about 98%, about 99%, or about 100% upon exposure to photostability challenge conditions (e.g., ultraviolet light for 1, 2, or 3 days at 25° C. and 60% RH; or white light for 7, 14, or 21 days at 25° C. and 60% RH). In particular embodiments, a product of chemical degradation of Form I of the sesquifumarate salt includes, one, two, or three of the following degradation products:

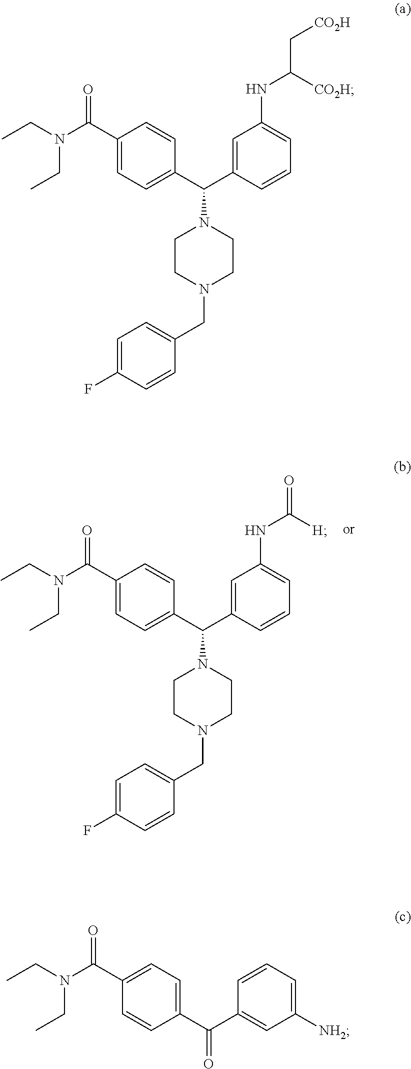

wherein (a) is a fumarate adduct impurity, (b) is a formamide impurity, and (c) is a des-piperazine impurity.

In certain embodiments, Form I of the sesquifumarate salt of Compound A1 exhibits characteristic physical stability parameters. For example, in certain embodiments, a sample of Form I of the sesquifumarate salt is physically stable (e.g., does not exhibit a substantial decrease in crystallinity and/or undergo crystal form change as observed by XRPD analysis) upon storage for 1 week at about 25° C. and about 60% RH. In certain embodiments, a sample of Form I of the sesquifumarate salt is physically stable (e.g., does not exhibit a substantial decrease in crystallinity and/or undergo crystal form change as observed by XRPD analysis) upon storage for 1 week at about 40° C. and about 75% RH. In certain embodiments, a sample of Form I of the sesquifumarate salt is physically stable (e.g., does not exhibit a substantial decrease in crystallinity and/or undergo crystal form change as observed by XRPD analysis) upon storage for 1 week at about 80° C. and about ambient humidity.

In certain embodiments, Form I of the sesquifumarate salt of Compound A1 is chemically pure. In certain embodiments, Form I of the sesquifumarate salt of Compound A1 is physically pure.

In certain embodiments, a sample of Form I of the sesquifumarate salt of Compound A1 contains birefringent particles comprising about 100%, about 90%, about 80%, about 70%, about 60%, or about 50% of the total number of particles in the sample.

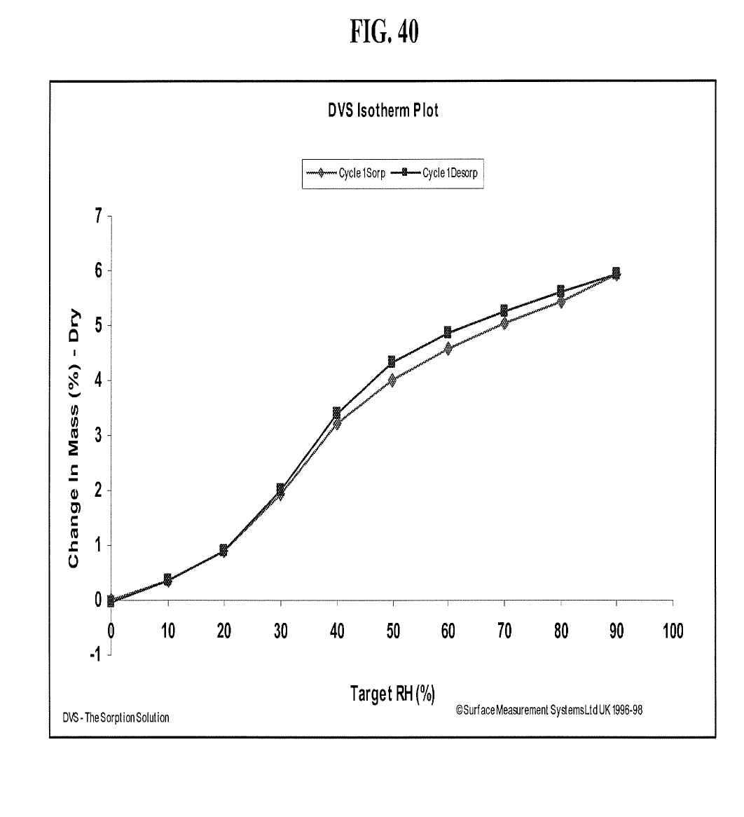

In certain embodiments, Form I of the sesquifumarate salt of Compound A1 exhibits particular characteristics with respect to moisture sorption. For example, in certain embodiments, Form I of the sesquifumarate salt of Compound A1 exhibits a moisture sorption profile matching the profile provided in FIG. 40. In certain embodiments, a sample of Form I of the sesquifumarate salt of Compound A1 gains less than about 10%, less than about 9%, less than about 8%, less than about 7%, less than about 6%, less than about 5%, less than about 4%, less than about 3%, less than about 2%, or less than about 1%, weight when increased from about 0% RH to about 90% RH at about ambient temperature. In certain embodiments, the weight gain is reversible upon decreasing from about 90% RH to about 0% RH at about ambient temperature.

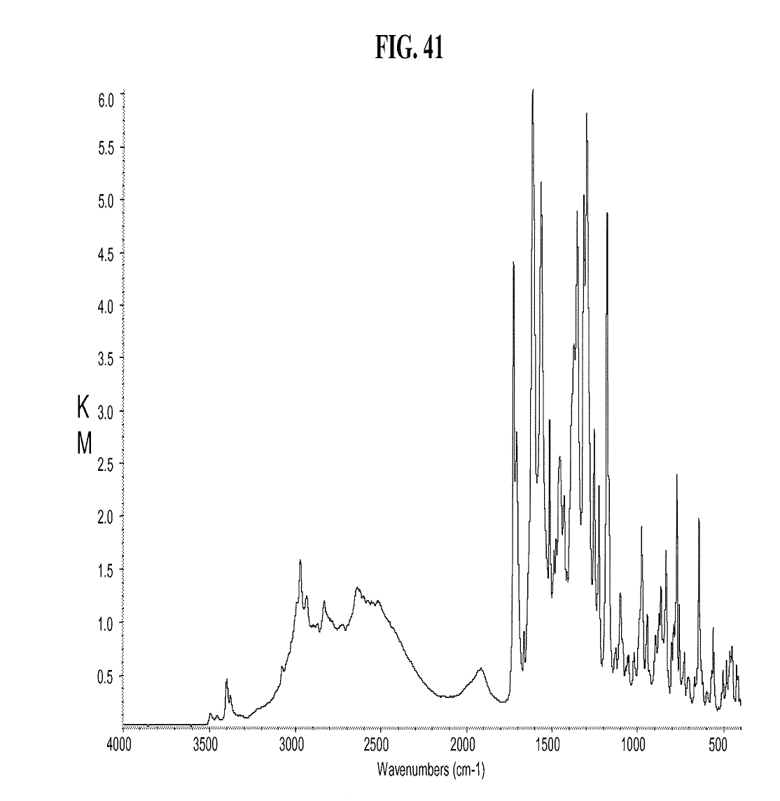

In certain embodiments, Form I of the sesquifumarate salt of Compound A1 exhibits particular characteristics with respect to infrared spectroscopy. For example, in certain embodiments, Form I of the sesquifumarate salt of Compound A1 exhibits an infrared spectrum matching the representative spectrum provided in FIG. 41. In certain embodiments, Form I of the sesquifumarate salt of Compound A1 exhibits an IR spectrum comprising any one, two, three, four, or five of the following peaks: about 2973 cm⁻¹, about 1725 cm⁻¹, about 1708 cm⁻¹, about 1613 cm⁻¹, and about 1563 cm⁻¹.

In certain embodiments, Form I of the sesquifumarate salt of Compound A1 exhibits particular characteristics with respect to solubility and/or dissolution. For example, in certain embodiments, Form I of the sesquifumarate salt has a solubility of between about 20 mg/ml and about 30 mg/ml (e.g., about 24 mg/ml) at ambient temperature in water with a pH of about 3.8. In certain embodiments, Form I of the sesquifumarate salt has a solubility of between about 5 mg/ml and 20 mg/ml (e.g., about 12 mg/ml) at ambient temperature in water with a pH of about 3.5 (0.1 M phosphoric acid). In certain embodiments, Form I of the sesquifumarate salt has a solubility of between about 10 mg/ml and 25 mg/ml (e.g., 17.4 mg/ml at 3 hr, 17.1 mg/ml at 24 hr) at ambient temperature in simulated gastric fluid with an initial pH of about 1.3. In certain embodiments, Form I of the sesquifumarate salt has a solubility of between about 1 mg/ml and about 10 mg/ml (e.g., 4.40 mg/ml at 3 hr, 4.13 mg/ml at 24 hr) at ambient temperature in fasted-state simulated intestinal fluid with an initial pH of about 6.51. In certain embodiments, Form I of the sesquifumarate salt has a solubility of between about 1 mg/ml and about 10 mg/ml (e.g., 2.54 mg/ml at 3 hr, 2.66 mg/ml at 24 hr) at ambient temperature in fed-state simulated intestinal fluid with an initial pH of about 5.03. In certain embodiments, Form I has a solubility of between about 0.01 and about 0.25 mg/ml (e.g., 0.18 mg/ml at 3 hr, 0.12 mg/ml at 24 hr) at ambient temperature in 0.1M phosphate buffer with an initial pH of about 7.5. In certain embodiments, Form I of the sesquifumarate salt has an intrinsic dissolution rate (IDR) of between about 250 μg/min/cm² and about 750 μg/min/cm² (e.g., 553 μg/min/cm²) at ambient temperature in fasted-state simulated intestinal fluid with an initial pH of about 6.51. In certain embodiments, Form I of the sesquifumarate salt has an IDR of between about 15 μg/min/cm² and about 50 μg/min/cm² (e.g., 33.5 μg/min/cm²) at ambient temperature in fed-state simulated intestinal fluid with an initial pH of about 5.03. In certain embodiments, Form I of the sesquifumarate salt at ambient temperature in simulated gastric fluid is too soluble to permit determination of its IDR in this medium.

In certain embodiments, Form I of the sesquifumarate salt can be obtained from any suitable laboratory solvent, including, but not limited to, solvent systems comprising ethyl acetate, acetonitrile, water, dichloromethane, petroleum ether, ethanol, toluene, isopropyl acetate, isopropanol, acetone, or a mixture of two or more thereof. In certain embodiments, the solvent system comprises a common laboratory solvent, as known in the art. In certain embodiments, Form I of the sesquifumarate salt of Compound A1 may be obtained by performing any three, four, five, six, or seven of the following steps: (a) obtain a first solution comprising the free base of Compound A1; (b) obtain a second solution comprising fumaric acid; (c) heat the first solution to a temperature above ambient temperature; (d) admix the first solution and the second solution such that the resulting mixture comprises approximately 1.5 molar equivalents of fumaric acid per mole of Compound A1; (e) cool the mixture to a temperature approximately equal to or below ambient temperature; (f) isolate Form I of the sesquifumarate salt of Compound A1; and (g) dry Form I of the sesquifumarate salt.

In certain embodiments, the first solution in step (a) comprises a common laboratory solvent, as known in the art. In a particular embodiment, the first solution in step (a) comprises ethanol and/or isopropyl acetate. In certain embodiments, the second solution in step (b) comprises a common laboratory solvent, as known in the art. In a particular embodiment, the second solution in step (b) comprises ethanol and/or isopropyl acetate. In certain embodiments, the temperature in step (c) is above about 25° C., above about 30° C., above about 40° C., above about 50° C., above about 60° C., or above about 70° C. In certain embodiments, the temperature in step (c) is about 35° C., about 40° C., about 45° C., about 50° C., about 55° C., about 60° C., about 65° C., about 70° C., or about 75° C. In certain embodiments, the resulting mixture in step (d) comprises about 1.00, about 1.05, about, about 1.10, about 1.15, about 1.20, about 1.25, about 1.30, about 1.35, about 1.40, about 1.45, about 1.50, about 1.55, about 1.60, about 1.65, about 1.70, about 1.75, about 1.80, about 1.85, about 1.90, about 1.95, or about 2.00 molar equivalents of fumaric acid per mole of Compound A1. In certain embodiments, the temperature in step (e) is about 25° C., about 20° C., about 15° C., about 10° C., about 5° C., about 0° C., or less than 0° C. In certain embodiments, the isolation in step (f) is comprises suction filtration. In certain embodiments, the drying in step (g) comprises vacuum drying. In certain embodiments, the drying in step (g) comprises drying at or below about 70° C., at or below about 60° C., at or below about 50° C., at or about 40° C., at below about 30° C., or at about ambient temperature.

In particular embodiments, Form I of the sesquifumarate salt of Compound A1 showed advantageous properties including properties relating to, e.g., thermal properties, physical stability, chemical stability, water uptake (e.g., hygroscopicity), solubility, dissolution, and solvent content.

### Pharmaceutical Compositions and Routes of Administration

Provided herein are pharmaceutical compositions comprising one or more Compound A1 solid form(s) as an active ingredient, in combination with one or more pharmaceutically acceptable excipient(s) or carrier(s). In certain embodiments, the pharmaceutical composition comprises at least one excipient or carrier.

Pharmaceutical compositions described herein may be administered by any route including, but not limited to, for example, orally, intramuscularly, subcutaneously, topically, intranasally, epidurally, intraperitoneally, intrathoracially, intravenously, intrathecally, intracerebroventricularly, and injecting into the joints.

In one embodiment, the route of administration is orally, intravenously or intramuscularly.

In one embodiment, the pharmaceutically acceptable carrier is selected from a solid carrier and a liquid carrier.

Solid carriers include, but are not limited to, for example, powders, tablets, dispersible granules, capsules, cachets, and suppositories. A solid carrier can be one or more substances, which may also act as diluents, flavoring agents, solubilizers, lubricants, suspending agents, binders, or table disintegrating agents. A solid carrier can also be an encapsulating material.

In powders, the carrier is a finely divided solid mixed with a finely divided compound of the invention.

In tablets, the compound of the invention is mixed with a carrier having the necessary binding properties and in proportions suitable to be compacted into the shape and size desired.

In a suppository, a low-melting wax, such as, for example, a mixture of fatty acid glycerides and cocoa butter is first melted and a compound of the invention is dispersed therein by, for example, stirring. The molten homogeneous mixture in then poured into convenient size molds and allowed to cool and solidify.

Suitable carriers, include but are not limited to, for example, magnesium carbonate, magnesium stearate, talc, lactose, sugar, pectin, dextrin, starch, tragacanth, methyl cellulose, sodium carboxymethyl cellulose, a low-melting wax, and cocoa butter.

The term “composition” is also intended to include the formulation of a compound of the invention with encapsulating material as a carrier to provide a capsule in which a compound of the invention (with or without other carriers) is surrounded by a carrier which is thus in association with it. Similarly, cachets are included.

Tablets, powders, cachets, and capsules can be used as solid dosage forms suitable for oral administration.

Liquid dosage forms include, but are not limited to, for example, solutions, suspensions, and emulsions. For example, sterile water or propylene glycol solutions of a compound of the invention may be liquid preparations suitable for parenteral administration. Liquid dosage forms can also be formulated as an aqueous polyethylene glycol solution.

Liquid dosage forms for oral administration can be prepared by dissolving a compound of the invention in water and adding suitable colorants, flavoring agents, stabilizers, and thickening agents as desired. Liquid suspensions for oral administration can be made by dispersing a finely divided compound of the invention in water together with a suspending agent, such as, for example, natural synthetic gums, resins, methyl cellulose, and sodium carboxymethyl cellulose.

One embodiment is directed to a pharmaceutical composition comprising from 0.05% to 99% w (percent by weight) of at least one compound of the invention, all percentages by weight being based on total composition.

Another embodiment is directed to a pharmaceutical composition comprising from 0.10 to 50% w (percent by weight) of at least one compound of the invention, all percentages by weight being based on total composition.

The pharmaceutical compositions provided herein may be formulated in various dosage forms for, e.g., oral, parenteral, or topical administration.

In certain embodiments, the pharmaceutical compositions provided herein may be administered at once or multiple times at intervals of time (e.g., once a day, twice a day, three times a day, or more times per day). An “effective amount” of Compound A1or salts or forms described herein may be determined by one of ordinary skill in the art, and includes exemplary dosage amounts for a mammal of from about 0.05 to about 300 mg/kg/day, preferably less than about 200 mg/kg/day, in a single dose or in or in the form of individual divided doses. Exemplary dosage amounts for an adult human are from about 1 to 100 (for example, 15) mg/kg of body weight of active compound per day, which can be administered in a single dose or in the form of individual divided doses, such as from 1 to 4 times per day.

The specific dose level and frequency of dosage for any particular subject, however, may vary and generally depends on a variety of factors, including, but not limited to, for example, the bioavailability of the compound(s) in accordance with the formula described herein in the administered form; metabolic stability and length of action of Compound A1or salts or forms described herein; species, age, body weight, general health, sex, and diet of the subject; mode and time of administration; rate of excretion; drug combination; and severity of the particular condition.

Certain embodiments provide methods of using at least one solid form comprising Compound A1 for treating, preventing, or managing at least one disease or disorder that can be treated, prevented, or managed by administration of a δ-opioid receptor ligand. Particular embodiments herein provide a method for treating, preventing or managing at least one disease or disorder that can be treated, prevented, or managed by administration of a δ-opioid receptor ligand to a warm-blooded animal in need thereof, comprising administering to said animal a therapeutically effective amount of a solid form comprising Compound A1.

Certain embodiments provide methods for treating, preventing or managing at least one disease or disorder that can be treated, prevented, or managed by administration of a δ-opioid receptor ligand to a warm-blooded animal in need thereof, comprising administering to said animal a pharmaceutical composition comprising a therapeutically effective amount of at least one solid form comprising Compound A1.

Certain embodiments provide using at least one solid form comprising Compound A1 in the manufacture of a medicament for treating, preventing or managing at least one disease or disorder that can be treated, prevented, or managed by administration of a δ-opioid receptor ligand.

Certain embodiments provide pharmaceutical compositions comprising a therapeutically effective amount of at least one solid form comprising Compound A1 for the treatment, prevention or management of at least one disease or disorder that can be treated, prevented, or managed by administration of a δ-opioid receptor ligand.

Certain embodiments provide a method for using at least one solid form comprising Compound A1 for treating, preventing, or managing at least one disease or disorder selected from depression, anxiety, pain, and AMDD.

Particular embodiments provide a method for treating, preventing, or managing AMDD in a warm-blooded animal in need of such treatment, prevention or management, comprising administering to said animal a therapeutically effective amount of at least one solid form comprising Compound A1.

Particular embodiments provide a method for treating, preventing, or managing depression in a warm-blooded animal in need of such treatment, prevention or management, comprising administering to said animal a therapeutically effective amount of at least one solid form comprising Compound A1.

Particular embodiments provide a method for treating, preventing, or managing anxiety in a warm-blooded animal in need of such treatment, prevention or management, comprising administering to said animal a therapeutically effective amount of at least one solid form comprising Compound A1.

Particular embodiments provide a method for treating, preventing, or managing pain in a warm-blooded animal in need of such treatment, prevention or management, comprising administering to said animal a therapeutically effective amount of at least one solid form comprising Compound A1.

Certain embodiments provide methods for treating, preventing or managing at least one disease or disorder selected from depression, anxiety, pain, and AMDD in a warm-blooded animal in need of such treatment, prevention or management, comprising administering to said animal a pharmaceutical composition comprising a therapeutically effective amount of at least one solid form comprising Compound A1.

Certain embodiments herein provide using at least one solid form comprising Compound A1 in the manufacture of a medicament for treating, preventing or managing at least one disease or disorder selected from depression, anxiety, pain, and AMDD.

Certain embodiments provide pharmaceutical compositions comprising a therapeutically effective amount of at least one solid form comprising Compound A1 for the treatment, prevention or management of at least one disease or disorder selected from depression, anxiety, pain, and AMDD.

In one embodiment, a warm-blooded animal is a mammalian species including, but not limited to, for example, humans and domestic animals, such as, for example, dogs, cats, and horses.

In a further embodiment, the warm-blooded animal is a human.

At least one solid form comprising Compound A1 described herein may be used for the manufacture of a medicament for the treatment of at least one psychiatric disorder described hereinbelow.

At least one solid form comprising Compound A1 described herein may be used for the manufacture of a medicament for the treatment of at least one disorder selected from pain, anxiety, depression, and AMDD.

At least one solid form comprising Compound A1 described herein may be used for the manufacture of a medicament for the treatment of pain.

At least one solid form comprising Compound A1 described herein may be used for the manufacture of a medicament for the treatment of anxiety.

At least one solid form comprising Compound A1 described herein may be used for the manufacture of a medicament for the treatment of depression.

At least one solid form comprising Compound A1 described herein may be used for the manufacture of a medicament for the treatment of AMDD.

At least one a solid form comprising Compound A1 described herein may be useful to treat at least one psychiatric disorder. Exemplary psychiatric disorders include, but are not limited to Schizophrenia and other Psychotic Disorder(s) such as, for example, Psychotic Disorder(s), Schizophrenia Disorder(s), Cognitive deficits in Schizophrenia (CDS), Schizoaffective Disorder(s), Delusional Disorder(s), Brief Psychotic Disorder(s), Shared Psychotic Disorder(s), Psychotic Disorder(s) Due to a General Medical Condition, Agitation associated with Schizophrenia, and Prevention of Suicide in Schizophrenia; Dementia and other Cognitive Disorder(s); Anxiety Disorder(s) such as, for example, Anxious Depression, Panic Disorder(s) Without Agoraphobia, Panic Disorder(s) With Agoraphobia, Agoraphobia Without History of Panic Disorder(s), Social Anxiety Disorder. Specific Phobia, Social Phobia, Obsessive-Compulsive Disorder(s), Stress related Disorder(s), Posttraumatic Stress Disorder(s), Acute Stress Disorder(s), Generalized Anxiety Disorder(s), and Generalized Anxiety Disorder(s) Due to a General Medical Condition; Mood Disorder(s) such as, for example, a) Depressive Disorder(s) (including but not limited to Major Depressive Disorder(s), Anxious Major Depressive Disorder, and Dysthymic Disorder(s)), b) Bipolar Depression and/or Bipolar mania (including but not limited to Bipolar I, including but not limited to those with manic, depressive or mixed episodes, and Bipolar II, Agitation associated with Bipolar Mania), c) Treatment Resistant Depression (TRD), d) Cyclothymiac's Disorder(s), and e) Mood Disorder(s) Due to a General Medical Condition, Seasonal Affect Disorder, and Irritability Associated with Autistic Disorder; Sleep Disorder(s); Disorder(s) Usually First Diagnosed in Infancy, Childhood, or Adolescence, such as, for example, Mental Retardation, Downs Syndrome, Learning Disorder(s), Motor Skills Disorder(s), Autism, Communication Disorders(s), Pervasive Developmental Disorder(s), Attention-Deficit and Disruptive Behavior Disorder(s), Feeding and Eating Disorder(s) of Infancy or Early Childhood, Tic Disorder(s), and Elimination Disorder(s); Substance-Related Disorder(s), such as, for example, Substance Dependence, Substance Abuse, Substance Intoxication, Substance Withdrawal, Alcohol-Related Disorder(s), Amphetamines (or Amphetamine-Like)-Related Disorder(s), Caffeine-Related Disorder(s), Cannabis-Related Disorder(s), Cocaine-Related Disorder(s), Hallucinogen-Related Disorder(s), Inhalant-Related Disorder(s), Nicotine-Related Disorder(s)s, Opioid-Related Disorder(s)s, Phencyclidine (or Phencyclidine-Like)-Related Disorder(s), and Sedative-, Hypnotic- or Anxiolytic-Related Disorder(s); Behavioral disorders associated with substance abuse or addiction; Attention-Deficit and Disruptive Behavior Disorder(s); Eating Disorder(s); Personality Disorder(s) such as, for example, Obsessive-Compulsive Personality Disorder(s); Impulse-Control Disorder(s); Tic Disorders, such as, for example, Tourette's Disorder, and Chronic motor or vocal tic disorder; and Transient Tic Disorder.

At least one of the above psychiatric disorders is defined, for example, in the American Psychiatric Association: Diagnostic and Statistical Manual of Mental Disorders, Fourth Edition, Text Revision, Washington, D.C., American Psychiatric Association, 2000.

In yet another embodiment, the solid forms provided herein, as well as pharmaceutical compositions and formulations thereof, may be administered concurrently, simultaneously, sequentially or separately with at least one other pharmaceutically active compound selected from the following:

(i) antidepressants, such as, for example, agomelatine, amitriptyline, amoxapine, bupropion, citalopram, clomipramine, desipramine, doxepin duloxetine, elzasonan, escitalopram, fluvoxamine, fluoxetine, gepirone, imipramine, ipsapirone, maprotiline, nortriptyline, nefazodone, paroxetine, phenelzine, protriptyline, ramelteon, reboxetine, robalzotan, sertraline, sibutramine, thionisoxetine, tranylcypromaine, trazodone, trimipramine, venlafaxine, and equivalents, and pharmaceutically active isomer(s) and metabolite(s) thereof;
(ii) atypical antipsychotics, such as, for example, quetiapine and pharmaceutically active isomer(s) and metabolite(s) thereof;
(iii) antipsychotics, such as, for example, amisulpride, aripiprazole, asenapine, benzisoxidil, bifeprunox, carbamazepine, clozapine, chlorpromazine, debenzapine, divalproex, duloxetine, eszopiclone, haloperidol, iloperidone, lamotrigine, loxapine, mesoridazine, olanzapine, paliperidone, perlapine, perphenazine, phenothiazine, phenylbutylpiperidine, pimozide, prochlorperazine, risperidone, sertindole, sulpiride, suproclone, suriclone, thioridazine, trifluoperazine, trimetozine, valproate, valproic acid, zopiclone, zotepine, ziprasidone, and equivalents, and pharmaceutically active isomer(s) and metabolite(s) thereof;
(iv) anxiolytics, such as, for example, alnespirone, azapirones, benzodiazepines, barbiturates such as adinazolam, alprazolam, balezepam, bentazepam, bromazepam, brotizolam, buspirone, clonazepam, clorazepate, chlordiazepoxide, cyprazepam, diazepam, diphenhydramine, estazolam, fenobam, flunitrazepam, flurazepam, fosazepam, lorazepam, lormetazepam, meprobamate, midazolam, nitrazepam, oxazepam, prazepam, quazepam, reclazepam, tracazolate, trepipam, temazepam, triazolam, uldazepam, zolazepam, and equivalents, and pharmaceutically active isomer(s) and metabolite(s) thereof;
(v) anticonvulsants, such as, for example, carbamazepine, valproate, lamotrogine, gabapentin and equivalents and pharmaceutically active isomer(s) and metabolite(s) thereof;
(vi) Alzheimer's therapies, such as, for example, donepezil, memantine, tacrine, and equivalents, and pharmaceutically active isomer(s) and metabolite(s) thereof;
(vii) Parkinson's therapies, such as, for example, deprenyl, L-dopa, Requip, Mirapex, MAOB inhibitors such as selegine and rasagiline, comP inhibitors such as Tasmar, A-2 inhibitors, dopamine reuptake inhibitors, NMDA antagonists, Nicotine agonists, Dopamine agonists and inhibitors of neuronal nitric oxide synthase, and equivalents, and pharmaceutically active isomer(s) and metabolite(s) thereof;
(viii) migraine therapies, such as, for example, almotriptan, amantadine, bromocriptine, butalbital, cabergoline, dichloralphenazone, eletriptan, frovatriptan, lisuride, naratriptan, pergolide, pramipexole, rizatriptan, ropinirole, sumatriptan, zolmitriptan, zomitriptan, and equivalents, and pharmaceutically active isomer(s) and metabolite(s) thereof;
(ix) stroke therapies, such as, for example, abciximab, activase, NXY-059, citicoline, crobenetine, desmoteplase, repinotan, traxoprodil and equivalents, and pharmaceutically active isomer(s) and metabolite(s) thereof;
(x) urinary incontinence therapies, such as, for example, darafenacin, falvoxate, oxybutynin, propiverine, robalzotan, solifenacin, tolterodine, and equivalents, and pharmaceutically active isomer(s) and metabolite(s) thereof;
(xi) neuropathic pain therapies, such as, for example, gabapentin, lidoderm, pregablin and equivalents, and pharmaceutically active isomer(s) and metabolite(s) thereof;
(xii) nociceptive pain therapies, such as, for example, celecoxib, etoricoxib, lumiracoxib, rofecoxib, valdecoxib, diclofenac, loxoprofen, naproxen, paracetamol, and equivalents, and pharmaceutically active isomer(s) and metabolite(s) thereof;
(xiii) insomnia therapies, such as, for example, agomelatine, allobarbital, alonimid, amobarbital, benzoctamine, butabarbital, capuride, chloral, cloperidone, clorethate, dexclamol, ethchlorvynol, etomidate, glutethimide, halazepam, hydroxyzine, mecloqualone, melatonin, mephobarbital, methaqualone, midaflur, nisobamate, pentobarbital, phenobarbital, propofol, ramelteon, roletamide, triclofos, secobarbital, zaleplon, zolpidem, and equivalents, and pharmaceutically active isomer(s) and metabolite(s) thereof; and
(xiv) mood stabilizers, such as, for example, carbamazepine, divalproex, gabapentin, lamotrigine, lithium, olanzapine, quetiapine, valproate, valproic acid, verapamil, and equivalents, and pharmaceutically active isomer(s) and metabolite(s) thereof.

When employed in combination with the solid forms provided herein, as well as pharmaceutical compositions and formulations thereof, the above other pharmaceutically active compound may be used, for example, in the amounts indicated in the Physicians' Desk Reference (PDR) or as otherwise determined by one of ordinary skill in the art.

### EXAMPLES

The following Examples are presented by way of illustration, not limitation.

### Example 1

### Compound A1 Synthesis (Example Route 1)

Compound A1 can be synthesized according to the general knowledge of one skilled in the art, in accordance with the following schemes (Schemes 1 and 2), and/or in accordance with the methods set forth herein (including those incorporated herein by reference). Solvents, temperatures, pressures, and other reaction conditions relating to this example synthetic route may be readily selected by one of ordinary skill in the art. Useful starting materials are commercially available and/or readily prepared by one skilled in the art. Combinatorial techniques can be employed in the preparation of compounds, for example, where the intermediates possess groups suitable for these techniques.

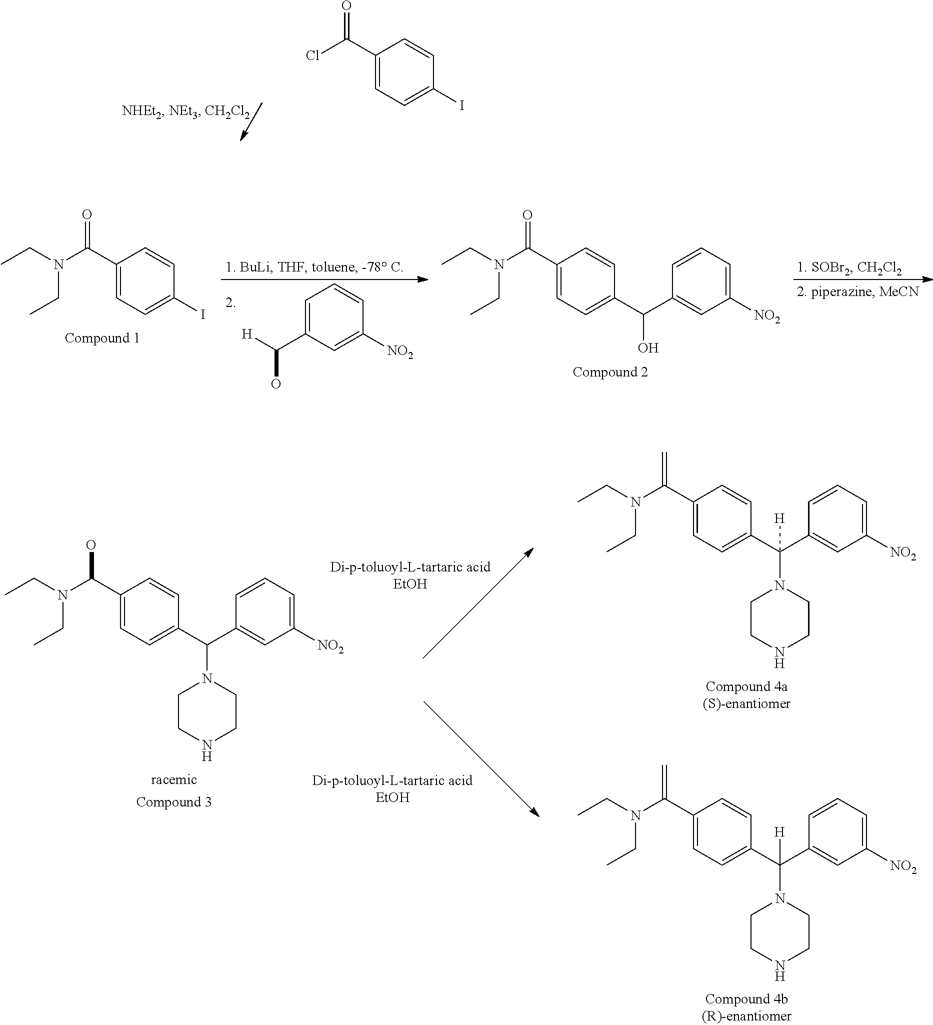

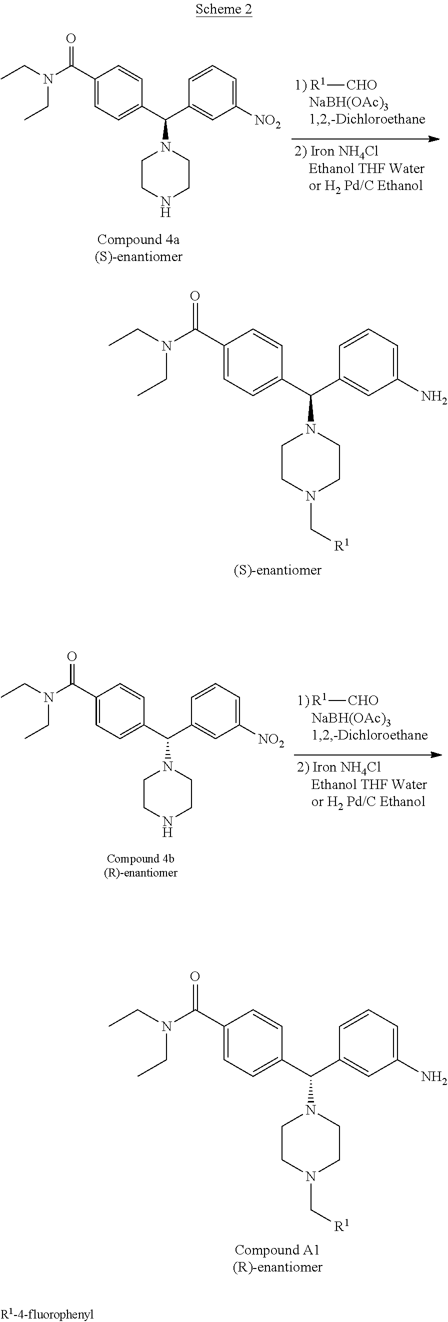

Compound 1: 4-iodo-N,N-diethylbenzamide. To a mixture of 4-iodo-benzoyl chloride (75 g) in 500 mL CH₂Cl₂ was added a mixture of Et₃N (50 mL) and Et₂NH (100 mL) at 0° C. After the addition, the resulting reaction mixture was warmed to RT in 1 h and was then washed with sat. NH₄Cl. The organic extract was dried (Na₂SO₄), filtered and concentrated. Residue was recrystallized from hot hexanes to give 80 g of Compound 1.

Compound 2: 4-[hydroxy(3-nitrophenyl)methyl]-N,N-diethylbenzamide. N,N-Diethyl-4-iodobenzamide (5.0 g, 16 mmol) was dissolved in THF (150 mL) and cooled to −78° C. under nitrogen atmosphere. n-BuLi (15 mL, 1.07 M solution in hexane, 16 mmol) was added dropwise during 10 min at −65 to −78° C. The solution was then cannulated into 3-nitrobenzaldehyde (2.4 g, 16 mmol) in toluene/THF (approx. 1:1, 100 mL) at −78° C. NH₄Cl (aq.) was added after 30 min. After concentration in vacuo, extraction with EtOAc/water, drying (MgSO₄) and evaporation of the organic phase, the residue was purified by chromatography on silica (0-75% EtOAc/heptane) to give Compound 2 (2.6 g, 50%). ¹H NMR (400 MHz, CDCl₃) δ_{H} 1.0-1.3 (m, 6H), 3.2 (m, 2H), 3.5 (m, 2H), 5.90 (s, 1H), 7.30-7.40 (m, 4H), 7.50 (m, 1H), 7.70 (d, J=8 Hz, 1H), 8.12 (m, 1H), 8.28 (m, 1H).

Compound 3: N,N-diethyl-4-[(3-nitrophenyl)(1-piperazinyl)methyl]benzamide. To a solution of Compound 2 (10.01 g, 30.5 mmol) in CH₂Cl₂ (200 mL) was added thionyl bromide (2.58 mL, 33.6 mmol). After 1 h at RT the reaction was washed with sat. aq. NaHCO₃ (100 mL) and the organic layer was separated. The aq. layer was washed with CH₂Cl₂ (3×100 mL) and the combined organic extracts were dried (Na₂SO₄), filtered, and concentrated. The crude benzyl bromide was dissolved in CH₃CN (350 mL) and piperazine (10.5 g, 122 mmol) was added. After heating the reaction for 1 h at 65° C. the reaction was washed with sat. NH₄Cl/EtOAc and the organic layer was separated. The aq. layer was extracted with EtOAc (3×100 mL) and the combined organic extracts were dried (Na₂SO₄), filtered and concentrated to give racemic Compound 3.

Compound 4b: N,N-diethyl-4-[(R)-(3-nitrophenyl)(1-piperazinyl)methyl]benzamide. Racemic Compound 3 was dissolved in EtOH (150 mL) and di-p-toluoyl-D-tartaric acid (11.79 g, 1 Eq.) was added. The product precipitated out over a 12 h period. The solid was collected by filtration and was redissolved in refluxing EtOH until all of the solid dissolved (approximately 1200 mL EtOH). Upon cooling, the solid was collected by filtration and the recrystallization repeated a second time. The solid was collected by filtration and was treated with aq. NaOH (2 M) and was extracted with EtOAc. The organic extract was then dried (Na₂SO₄), filtered and concentrated to give 1.986 g of Compound 4b. ¹H NMR (400 MHz, CDCl₃) δ_{H} 1.11 (br s, 3H), 1.25 (br s, 3H), 2.37 (br s, 4H), 2.91 (t, J=5 Hz, 4H), 3.23 (br s, 2H), 3.52 (br s, 2H), 4.38 (s, 1H), 7.31-7.33 (m, 2H), 7.41-7.43 (m, 2H), 7.47 (t, J=8 Hz, 1H), 7.75-7.79 (m, 1H), 8.06-8.09 (m, 1H), 8.30-8.32 (m, 1H).

### Compound A1: 4-{(R)-(3-aminophenyl)[4-(4-fluorobenzyl)piperazin-1-yl]methyl}-N,N-diethylbenzamide

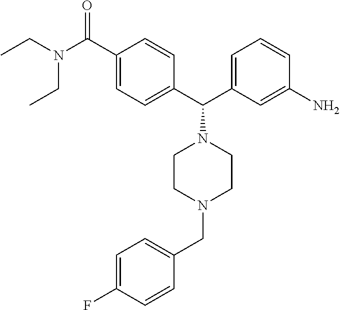

To a solution of Compound 4b (5.790 g, 14.6 mmol) in 1,2-dichloroethane (60 mL) was added 4-fluorobenzaldehyde (2.04 mL, 19.0 mmol) and sodium triacetoxy borohydride (4.02 g, 19.0 mmol). After 20 h at RT the reaction was quenched with aq. NaHCO₃ and the organic layer was separated. The aq. layer was extracted with CH₂Cl₂ (3×100 mL) and the combined organic extracts were dried (Na₂SO₄), filtered and concentrated. The residue was purified by flash chromatography, eluting 30%-50% acetone in hexanes to afford a colorless foam (5.285 g, 71%), which is the nitro intermediate. The nitro intermediate (5.285 g, 10.4 mmol) was dissolved in a mixture of EtOH, THF, water and aq. sat. NH₄Cl (4:2:1:1 ratio v/v) (100 mL) and granules of iron (0.63 mg, 11.5 mmol) were added. The reaction was heated to reflux and periodically more iron granules were added. After 24 h at reflux (90° C.) the reaction was cooled to RT and filtered through celite and concentrated. To the residue was added aq. NaHCO₃ and CH₂Cl₂. The organic layer was separated and the aq. layer was extracted with CH₂Cl₂ (3×100 mL) and the combined organic extracts were dried (Na₂SO₄), filtered and concentrated. The product was purified on silica gel, eluting 1%-5% MeOH in CH₂Cl₂ to afford 3.505 g Compound A1 as a pale yellow foam. Impure material was additionally obtained from the above flash chromatography. The impure material was repurified by a second flash chromatography, eluting 100% EtOAc to 5% MeOH in EtOAc to yield a further 0.949 g of Compound A1.

Combined material obtained: 4.454 g (90% yield). Purity (HPLC): >99%; Optical purity (Chiral HPLC): >99%; ¹H NMR (400 MHz, CD₃OD), 1.08 (t, J=6.5 Hz, 3H), 1.21 (t, J=6.5 Hz, 3H), 3.20-3.26 (m, 4H), 3.51-3.54 (m, 6H), 4.43 (s, 2H), 7.19-7.23 (m, 2H), 7.34 (d, J=8.0 Hz, 1H), 7.40 (d, J=8.0 Hz, 2H), 7.54-7.63 (m, 3H), 7.70-7.82 (m, 4H). Found: C, 54.00; H, 6.34; N, 8.47. C₂₉H₃₅FN4O×4.7 HCl×0.2 C₄H₁₀O×0.1 H₂O has C, 54.02; H, 6.37; N, 8.46%. Dissociation constant: pKa=7.26 (piperazine nitrogen of free base). Partition coefficient: LogD (pH 7.4)=2.53 (free base).

### Example 2

### Compound A1 Synthesis (Example Route 2)

Compound A1 can be synthesized according to the general knowledge of one skilled in the art, in accordance with the following scheme (Scheme 3), and/or in accordance with the methods set forth herein (including those incorporated herein by reference). Solvents, temperatures, pressures, and other reaction conditions relating to this example synthetic route may be readily selected by one of ordinary skill in the art. Useful starting materials are commercially available and/or readily prepared by one skilled in the art. Combinatorial techniques can be employed in the preparation of compounds, for example, where the intermediates possess groups suitable for these techniques.

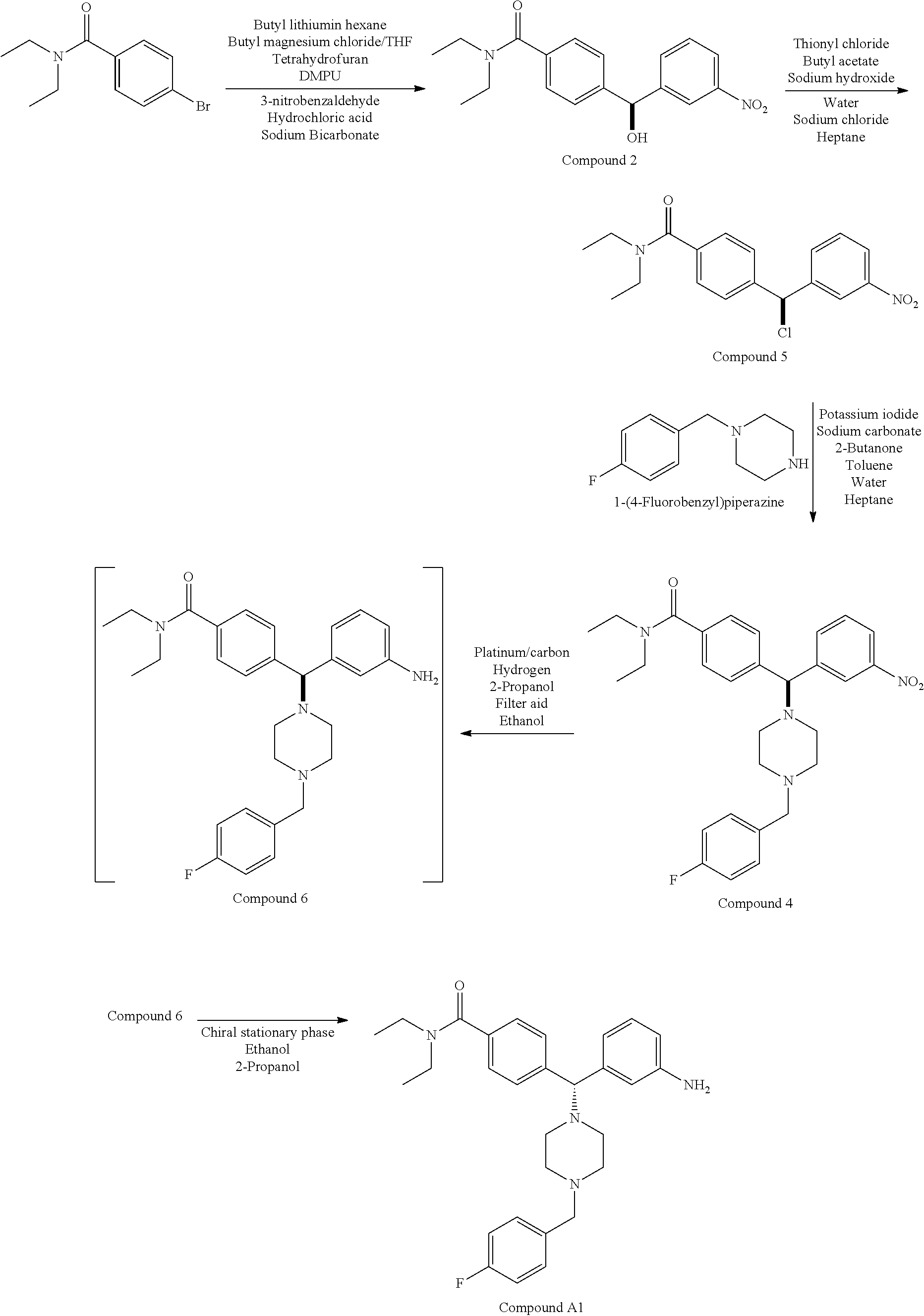

Compound 2: N,N-Diethyl-4-[hydroxy(3-nitrophenyl)methyl]benzamide. n-BuLi (234 mL, 2.5 M solution in hexanes, 585 mmol) was added to a solution of THF (300 mL) and n-BuMgCl (146 mL, 292 mmol, 2M in THF) at −10 to −5° C. over a period of 60 min and the resulting solution was stirred for a further 30 min. The resulting solution was cooled to −40° C. A solution of N,N-diethyl-4-bromobenzamide (150 g, 586 mmol) in THF (150 mL) was then added over 90 min, and following complete addition the reaction mixture was held for 30 min before being warmed to −25 to −20° C. 1,3-dimethyl-3,4,5,6-tetrahydro-2(1H)-pyrimidinone (150 mL) was added, followed the addition of a solution of 3-nitrobenzaldehyde (79.7 g, 527 mmol) in THF (150 mL) over at least 60 min. The reaction was held for 60 min before concentrated HCl (103 g of 36-38% w/w) in water (131 mL) was added and the reaction was allowed to warm to room temperature. The lower aqueous phase was discarded and the organic layer was washed with aqueous NaHCO₃ (7.1 g in 150 mL), followed by water (150 mL). The residue was purified by flash chromatography on silica gel eluting with toluene and then butyl acetate to provide Compound 2 (129.5 g, 67%). Analysis: ¹H NMR (400 MHz, DMSO-d₆) δ ppm 1.06 (m, 6H) 3.16 (m, 2H) 3.39 (m, 2H) 5.91 (d, J=4.2 Hz, 1H) 6.30 (d, J=4.2 Hz, 1H) 7.29 (d, J=8.2 Hz, 2H) 7.45 (d, J=8.2 Hz, 2H) 7.61 (t, J=8.1 Hz, 1H) 7.82 (m, 1H) 8.09 (ddd, J=8.1, 2.4, 1.1 Hz, 1H) 8.26 (m, 1H).

Compound 5: 4-[Chloro(3-nitrophenyl)methyl]-N,N-diethylbenzamide. To a solution of Compound 2 (150 g, 457 mmol) in butylacetate (1125 mL) at 35° C. was added thionyl chloride (70.19 g, 590 mmol) in butylacetate (75 mL) over approximately 60 min and the reaction was subsequently held for 30 min at 40-45° C. The reaction mixture was cooled to <5° C. before a solution of NaOH (68.85 g, 1.723 mol) in water (356 mL) was added over approximately 1 hr. The separate organic layer was washed with water (300 mL) and a solution of sodium chloride (105 g in 300 mL of water). The organic solution was concentrated before heptane (700 mL) was added. Initial crystallization of the product occurred at 50° C. and the slurry was cooled to <5° C. The product was washed with heptane (2×150 mL) and dried under vacuum (143 g, 90%). Analysis: ¹H NMR (400 MHz, DMSO-d₆) δ ppm 1.07 (m, 6H) 3.14 (m, 2H) 3.40 (m, 2H) 6.79 (s, 1H) 7.38 (d, J=8.4 Hz, 2H) 7.55 (d, J=8.4 Hz, 2H) 7.71 (t, J=8.1 Hz, 1H) 7.95 (m, 1H) 8.19 (ddd, J=8.1, 2.3, 1.1 Hz, 1H) 8.34 (m, 1H).

Compound 4: N,N-Diethyl-4-[[4-(4-fluorobenzyl)piperazin-1-yl](3-nitrophenyl)methyl]benzamide. Compound 5 (200 g, 577 mmol), sodium carbonate (140.4 g, 1.324 mol), 1-(4-fluorobenzyl)piperazine (140.4 g 723 mmol) and potassium iodide (9.20 g, 55 mmol) were slurried in butanone (600 mL), heated to reflux, and held at these conditions for >23 hours. Toluene (200 mL) was added to the vessel and the temperature adjusted to 77° C. before water (600 mL) was added. The layers were separated and additional water (100 mL) and toluene (400 mL) were added to the retained organic layer. Following separation of the layers, the organic layer was distilled to removed (800 mL) of solvent. The residual solution was treated with n-heptane (400 mL) at 90° C. Following further cooling (isolated at <5°) and seeding, the product was crystallized, isolated, washed with n-heptane (200 mL), and dried to provide Compound 4 (239.1 g, 82%). Analysis: ¹H NMR (400 MHz, DMSO-d₆) δ ppm 1.04 (m, 6H) 2.23-2.46 (m, 8H) 3.12 (m, 2H) 3.37 (m, 2H) 3.43 (s, 2H) 4.61 (s, 1H) 7.08 (t, J=8.8 Hz, 2H) 7.27 (m, 4H) 7.46 (d, J=8.0 Hz, 2H) 7.60 (dd, J=8.1, 7.8 Hz, 1H) 7.86 (m, 1H) 8.05 (dd, J=8.1, 2.3 Hz, 1H) 8.25 (m, 1H).

Compound 6: 4-{(3-Aminophenyl)[4-(4-fluorobenzyl)piperazin-1-yl]methyl}-N,N-diethylbenzamide. Compound 4 (200 g, 396 mmol) and 5% platinum on carbon, 50% water wet (31.2 g at 2.5% w/w, 4.0 mmol) in isopropanol (1600 mL) were slurried at 40° C. and subjected to hydrogenation conditions for >6 hours. The reaction was then cooled to 25° C., filtered through Harborlite® (50 g), and washed through with isopropanol (400 mL), and the resulting solution was concentrated by distillation (removing 1.04 L of solvent) and then diluted with ethanol (890 mL) to provide Compound 6 as a solution (approx. 10% w/v in 1:1 isopropanol:ethanol containing 191 g of product, 101% yield). Analysis: ¹H NMR (400 MHz, DMSO-d₆) δ ppm 1.04 (m, 6H) 2.25-2.43 (m, 8H) 3.29 (m, 4H) 3.45 (s, 2H) 4.13 (s, 1H) 4.77 (s, 2H) 6.39 (dd, J=7.9, 1.8 Hz, 1H) 6.56 (m, 1H) 6.63 (m, 1H) 6.91 (t, J=7.9 Hz, 1H) 7.06 (dd, J=8.8, 8.4 Hz, 2H) 7.23 (d, J=8.2 Hz, 2H) 7.28 (dd, J=8.4, 5.8 Hz, 2H) 7.41 (d, J=8.2 Hz, 2H). Also contained IPA: 1.05 (d, J=6.3 Hz, 6H) 3.79 (m, 1H).

Compound A1: 4-{(R)-(3-Aminophenyl)[4-(4-fluorobenzyl)piperazin-1-yl]methyl}-N,N-diethylbenzamide. A 10% w/w solution of Compound 6 (18.5 kg, 3.9 mol) was purified by chiral chromatography using Chiralpak® AD 20 micron eluting with ethanol:isopropanol (1:1). The fractions containing the correct enantiomer were concentrated to provide Compound A1 (522 g, 28.2%). Analysis: ¹H NMR (400 MHz, DMSO-d₆) δ ppm 1.05 (m, 6H) 2.17-2.46 (m, 8H) 3.15 (m, 2H) 3.36 (m, 2H) 3.43 (s, 2H) 4.07 (s, 1H) 4.99 (s, 2H) 6.36 (ddd, J=7.8, 2.2, 0.9 Hz, 1H) 6.54 (m, 1H) 6.61 (m, 1H) 6.90 (t, J=7.8 Hz, 1H) 7.09 (dd, J=8.9, 8.6 Hz, 2H) 7.24 (d, J=8.2 Hz, 2H) 7.28 (dd, J=8.6, 5.8 Hz, 2H) 7.41 (d, J=8.2 Hz, 2H). Also contained IPA: 1.03 (d, J=6.3 Hz, 6H) 3.77 (m, 1H) & EtOH: 1.05 (t, J=6.9 Hz, 3H) 3.45 (q, J=6.9 Hz, 2H). In some cases, Compound A1 material is not isolated as a solid from the concentrated fractions, but instead carried through (with further concentration, if necessary) for use in salt formation, as described herein.

### Example 3

### Analytical Determination of Enantiomeric Purity by Capillary Electrophoresis

Analytical Method

Instrument: Beckman Coulter® P/ACE MDQ

Capillary: Polyimide-coated fused silica capillary tubing 50 μm, 365 μm OD

Capillary length: 60 cm total length, 50 cm effective length

Separation buffer: Triethanolamine phosphate (pH 2.5, 100 mM) containing 10 mM hydroxyethyl-β-cyclodextrin (Aldrich®)

Detection: UV at 200 nm (detection aperture 800×100 μm)

Capillary temp: 20° C.

Voltage: +30 kV (ramping from 0 to 30 kV over 0.3 min)

Current draw: 60 μA approx.

Capillary preconditioning: 1 min at 100 psi with separation buffer

Injection: 10 s at 0.5 psi (hydrodynamic, capillary inlet at anode)

Run time: 22 min

### Migration Data

Approximate migration times for enantiomers of Compound A1:

S-enantiomer: 18.2 min approx. migration time; RMT=0.98

R-enantiomer: 18.6 min approx. migration time; RMT=1.00

### Solution Preparation

Blank (Diluent): Methanol:Water, 5:95 (v:v).

SST: Weigh approximately 2 mg of racemic Compound A1 into a 20 ml flask. Add 1 ml of methanol, ensure complete dissolution and dilute to volume with water.

1.0% Standard Solution: Add 1.0 ml of the SST solution to a 100 ml flask and dilute to volume with diluent.

Sample: Obtain an aliquot of sample containing approximately 2 mg of Compound A1 material for enantiomeric purity analysis. Place into a 20 ml flask and dilute to volume with diluent.

### Analytical Procedure

Analyze the blank, SST and 1.0% standard solutions. Integrate only the peaks corresponding to the enantiomers. (The recovery of the 1.0% standard should be within 50-150% of the SST.) Calculate the enantiomeric excess (ee) using the following equation: ee (% with respect to R)=Area % of R-enantiomer−Area % of S-enantiomer.

### Example 4

### Methods and Techniques for Solid Form Synthesis and Analysis

### Solubility Measurements

A weighed sample is treated with aliquots of the test solvent at room temperature or elevated temperature. Complete dissolution of the test material is determined by visual inspection. Solubility is estimated based on the total solvent used to provide complete dissolution. The actual solubility may be greater than the value calculated because of the use of solvent aliquots that were too large or due to a slow rate of dissolution. The solubility is expressed as “less than” if dissolution did not occur during the experiment. If complete dissolution is achieved as a result of only one aliquot addition, the solubility is expressed as “greater than”. Solubility of a salt may be expressed as the mass of salt dissolved per unit volume of solution, (denoted as, for example, “mg/mL”), or as the free base equivalent (denoted as, for example, “mgA/mL”).

### Salt Preparation

Experimental conditions include different acids, solvents, stoichiometries and crystallization techniques. These techniques are described in more detail herein. Once solid samples are harvested from salt attempts, they are either examined under a microscope for birefringence and morphology or observed with the naked eye. Any crystalline shape is noted. Solid samples are then analyzed by XRPD.

Crash Precipitation. A solution containing the free base and an acid is prepared at elevated temperature. The solution is then filtered into an antisolvent at ambient temperature. The resulting solids are isolated and analyzed.

Crash Cool. Solutions containing the free base and an acid are prepared in various solvents at elevated temperature. Vials are placed in a refrigerator or a freezer. The resulting solids are isolated by vacuum filtration and analyzed by XRPD.

Fast Evaporation. A solution containing free base and an acid is prepared and filtered. The filtered solution is allowed to evaporate at ambient in an uncapped vial. Solids are isolated and analyzed.

Slow Cool. A solution of the free base and an acid is prepared at elevated temperature. The mixture is then allowed to cool down to room temperature. The presence or absence of solids is noted. If there are no solids present, the vial is placed in a refrigerator. The presence or absence of solids is noted and any resulting solid is isolated and analyzed.

Slurry. Solutions are prepared by adding an acid solution to a solution of free base with excess solids present. The mixture is then agitated in a sealed vial at ambient or elevated temperature for a given amount of time. Solids are isolated and analyzed.

### Solid Form Preparation

Both thermodynamic and kinetic crystallization techniques are employed. These techniques are described in more detail herein. Once solid samples are harvested from crystallization attempts, they are either examined under a microscope for birefringence and morphology or observed with the naked eye. Any crystalline shape is noted. Solid samples are then analyzed by XRPD, and the crystalline patterns compared to each other to identify new crystal forms.

Cold Precipitation. Solutions are prepared in various solvents at elevated temperature. The solutions are then filtered into an antisolvent at sub-ambient temperature. If there are no solids present, or if the amount of solids is too small for XRPD analysis, the vial is placed in a freezer. The resulting solids are isolated by filtration and analyzed.

Crash Cool. Saturated solutions are prepared in various solvents at elevated temperatures and filtered into a vial while still warm. Vials are then placed in a refrigerator or an ice bath. The resulting solids are isolated by filtration and analyzed.

Fast Evaporation. Solutions are prepared in various solvents and sonicated between aliquot additions to assist in dissolution. Once a mixture reaches complete dissolution, as judged by visual observation, the solution is filtered. The filtered solution is allowed to evaporate at ambient. The solids that form are isolated and analyzed.

Freeze Dry. Dilute water solution is prepared, filtered, and frozen. The frozen sample is lyophilized.

Slow Evaporation. Solutions are prepared in various solvents and sonicated between aliquot additions to assist in dissolution. Once a mixture reaches complete dissolution, as judged by visual observation, the solution is filtered. The filtered solution is allowed to evaporate. The solids that form are isolated and analyzed.

Grinding. A solid sample is placed into a stainless steel milling rotor with a small metal ball. The sample is ground on a ball mill for a given amount of time. The solids are isolated and analyzed.

Slow Cool. Saturated solutions are prepared in various solvents at elevated temperatures and filtered while still warm. The vial is covered and allowed to cool slowly to room temperature. The presence or absence of solids is noted. If there are no solids present, or if the amount of solids is too small for XRPD analysis, the vial is placed in a refrigerator overnight. Again, the presence or absence of solids is noted and if there are none, the vial is placed in a freezer overnight. Solids that form are isolated by filtration and allowed to dry prior to analysis.

Slurry Experiments. Solutions are prepared by adding enough solids to a given solvent so that excess solids are present. The mixture is then agitated in a sealed vial at either ambient or elevated temperature. After a given amount of time, the solids are isolated by vacuum filtration.

Stress Experiments. Solids are stressed under different temperature or relative humidity (RH) environments for a measured time period. Specific RH values are achieved by placing the sample inside sealed chambers containing saturated salt solutions, as known in the art. The salt solutions are selected and prepared following an ASTM standard procedure. Samples are analyzed by XRPD immediately after removal from the stress environment.

Vapor Diffusion Experiments. A solid sample is placed for a predetermined period of time in a stability chamber having a particular humidity level (controlled, e.g., with a saturated salt solution). The sample is analyzed by XRPD immediately after removal from the chamber.

### Instrumental Techniques

The solid forms provided herein are characterized by any suitable analytical instrumentation and method known to a person of ordinary skill in the art. Examples of suitable analytical instrumentation and methods are provided herein.

### High Performance Liquid Chromatography (HPLC)

Analyses are carried out on an Agilent® model 1100 high performance liquid chromatograph using gradient elution on a reversed phase column utilizing ultraviolet (UV) detection. Approximately 25 mg of the sample is weighed accurately and dissolved in methanol/water/trifluoroacetic acid (35:65:0.05) to a volume of 50 mL. The sample solution is analysed using the conditions specified below:

| Item | Condition | | |
|---|---|---|---|
| Column | 150 mm × 4.6 mm I.D. stainless steel packed | | |
| | with SymmetryShield RP8, 3.5 μm particle size, | | |
| | or equivalent. | | |
| Mobile phase | Mobile Phase A: Water/TFA, 1000:1 | | |
| | Mobile Phase B: Acetonitrile/TFA, 1000:1 | | |
| | | % Mobile | % Mobile |
| Gradient | Time (min) | Phase A | Phase B |
| | 0 | 90 | 10 |
| | 35 | 50 | 50 |
| | 40 | 10 | 90 |
| | 40.1 | 90 | 10 |

| | |
|---|---|
| Detector wavelength | 254 nm |
| Column temperature | 40° C. |
| Flow rate | 1.50 mL/min |
| Injection volume | 10 μL |
| Post-time | 5 minutes |
| Run time | 40 minutes |
| (data collection) | |

The content of the drug and related substances are quantified by area (%).

### Gas Chromatography (GC)

Analyses are carried out using an Agilent® model 6890 gas chromatograph with split/splitless injector in split mode and flame ionization detection. A solution containing 30 mg/mL of sample and 5 mg/mL decane as internal standard is prepared using dimethylformamide as solvent. The sample is analysed using the conditions specified below:

| Oven | Temperature | 40° C. |
|---|---|---|
| | Initial Time | 5.00 min |
| | Rate | 10° C./min |
| | Final temp | 260° C. |
| | Final time | 3 min |
| Injector | Injection volume | 1 μL |
| | Mode | split |
| | Temp | 250° C. |
| | Pressure | 3 psig |
| | Split ratio | 10:1 |
| | Split flow | 40.8 mL/min |
| | Total flow | 47.2 mL/min |
| Column | 30 m × 0.53 mm ID fused silica DB-1 capillary | |
| | (3μ film thickness) | |
| | Carrier | He |
| | Mode | Constant pressure |
| | Pressure | Approximately 3.1 psig |
| | Flow | Approximately 30 cm/sec at 40° C. |
| Detector | Temperature | 300° C. |
| | H₂ flow | 30 mL/min |
| | Air flow | 300 mL/min |
| | Mode | Constant make-up |
| | Make-up (N₂) | 30 mL/min |

The residual solvents content is quantified with reference to the internal standard.

### Ion Chromatography

Chloride content is determined by ion chromatography using a Dionex® DX-500 modular ion chromatography system. Approximately 10 mg of sample is weighed accurately into a 50 mL volumetric flask, dissolved initially in 2 mL of water containing 200 μL methanol then diluted to volume with water to give a solution having an expected chloride content of approximately 15 ppm. The sample is analysed using the conditions specified below:

| | |
|---|---|
| Column | Dionex ® IonPac AS11-HC 25 cm × 2.0 mm |
| Eluent | 1 mM KOH |
| Overall run time | 38 mins including equilibration |
| Flow rate | 1.0 mL min⁻¹ |
| Detection² | Suppressed Conductivity |
| Injection volume | 25 μL |
| Column temperature | 30° C. |
| Suppressor | ASRS 300 |

The chloride content is quantified by comparison to external standards of known concentration.

### Intrinsic Dissolution Rate

Intrinsic dissolution rate is determined at 37° C. using a Distek Dissolution System model 5100A with a sample size of approximately 200 mg (0.5 cm² disc) and 500 mL dissolution medium. The paddle speed is set to 500 rpm and the paddle height to approximately 2.54 cm above the disc surface. The concentration of sample in solution is determined by HPLC.

### Differential Scanning Calorimetry (DSC)

Analyses are carried out on a TA Instruments differential scanning calorimeter 2920. The instrument is calibrated using indium as the reference material. The sample is placed into a standard aluminum DSC pan with an uncrimped or a crimped lid configuration, and the weight accurately recorded. The sample cell is equilibrated at about 25° C. and heated under a nitrogen purge at a rate of about 10° C./min. To determine the glass transition temperature (Tg) of amorphous material, the sample cell is cycled several times. The Tg is reported from the inflection point of the transitions as the average value.

### Dynamic Vapor Sorption/Desorption (DVS)

Data are collected on a VTI SGA-100 moisture balance system. For sorption isotherms, a sorption range of about 0% to about 90% relative humidity (RH) and a desorption range of about 90 to about 0% RH in 10% RH increments are used for analysis. The samples are not dried prior to analysis. Equilibrium criteria used for analysis are less than 0.0100% weight change in 5 minutes with a maximum equilibration time of 3 hours if the weight criterion is not met. Data are not corrected for the initial moisture content of the samples.

### Modulated Differential Scanning Calorimetry (MDSC)

Modulated differential scanning calorimetry data are obtained on a TA Instruments differential scanning calorimeter 2920 equipped with a refrigerated cooling system (RCS). The sample is placed into an aluminum DSC pan, and the weight is accurately recorded. The pan is covered with a lid with is either crimped or uncrimped. MDSC data are obtained using a modulation amplitude of +/−0.8° C. and a 60 second period with an underlying heating rate of about 2° C./min. The temperature and the heat capacity are calibrated using indium metal and sapphire as the calibration standards, respectively. The reported glass transition temperature is obtained from the inflection of the step change in the reversible heat flow versus temperature curve.

### Nuclear Magnetic Resonance (NMR)

NMR spectroscopy is carried out using a Bruker Avance 1 500 MHz spectrometer. The solution phase ¹H and ¹³C NMR spectra are acquired at about 500 and 125 MHz respectively, at about 300K, in hexadeuterated dimethylsulfoxide with added tetramethylsilane (TMS) for reference. The ¹H spectrum is referenced to tetramethylsilane (0.00 ppm) and the ¹³C spectrum is referenced to the NMR solvent (39.5 ppm).

### Optical Microscopy

Observations made by optical microscopy are collected on a Wolfe polarized light microscope with magnification. Crossed polarizers are used to observe birefringence in the samples.

### Thermogravimetry (TG)

Analyses are carried out on a TA Instruments 2950 thermogravimetric analyzer. The calibration standards are nickel and Alumel™. Each sample is placed in an aluminum sample pan and inserted into the TG furnace. Samples are first equilibrated at about 25° C. or started directly from ambient conditions, then heated under a stream of nitrogen at a heating rate of about 10° C./min.

### Thermogravimetric Infrared (TG-IR)

Thermogravimetric infrared (TG-IR) analyses are acquired on a TA Instruments thermogravimetric (TG) analyzer model 2050 interfaced to a Magna 560® Fourier transform infrared (FT-IR) spectrophotometer (Thermo Nicolet) equipped with an Ever-Glo mid/far IR source, a potassium bromide (KBr) beamsplitter, and a deuterated triglycine sulfate (DTGS) detector. The TG instrument is operated under a flow of helium at 90 and 10 cc/min for the purge and balance, respectively. Each sample is placed in a platinum sample pan, inserted into the TG furnace, accurately weighed by the instrument, and the furnace is heated from ambient temperature at a rate of about 20° C./min. The TG instrument is started first, immediately followed by the FT-IR instrument. Each IR spectrum represents co-added scans collected at a spectral resolution of about 4 cm⁻¹. IR spectra are collected about every 1 s. A background scan is collected before the beginning of the experiment. Wavelength calibration is performed using polystyrene. The TG calibration standards are nickel and Alumel™ Volatiles are identified from searching a relevant spectral library.

### X-Ray Powder Diffraction (XRPD)

X-ray powder diffraction analyses are performed using a Philips X'Pert PRO diffractometer, equipped with a RTMS detector. Data are collected at about 25° C. using Cu Kα radiation starting at approximately 2°2θ with a step size of about 0.020°2θ and a step time of about 2 s. The tube voltage and amperage are set to 40 kV and 30 mA, respectively. Patterns are displayed from about 2 to about 40°2θ. Samples are prepared for analysis by packing them into thin-walled glass capillaries. Each capillary is mounted onto a goniometer head that is motorized to permit spinning of the capillary during data acquisition. Instrument calibration is performed using a silicon reference standard.

XRPD peak positions are obtained either by visual inspection of XRPD patterns or using the software Pattern Match version 2.3.5 (see Ivanisevic, I. et al., System and method for matching diffraction patterns, U.S. Patent App. Pub. No. 20040103130, May 2004). In general, positions of individual XRPD peaks are expected to vary by about ±0.2°2θ from sample to sample. Determination of whether an XRPD pattern matches a second XRPD pattern is performed either by visual inspection of the two XRPD patterns or using the software Pattern Match version 2.3.5. In general, as understood in the art, two XRPD patterns match one another if the characteristic peaks of the first pattern are located at approximately the same positions as the characteristic peaks of the second pattern. As understood in the art, determining whether two XRPD patterns match may require consideration of individual variables and parameters such as, but not limited to, preferred orientation, phase impurities, degree of crystallinity, particle size, variation in diffractometer instrument setup, variation in XRPD data collection parameters, and variation in XRPD data processing, among others.

### Example 5

### Solid Forms Comprising Compound A1

### Salt Preparation Methodology

Salt Preparation. Possible salts of the free base of Compound A1 were investigated and their properties were characterized.

A 0.03 M solution of Compound A1 free base in MeOH was prepared. 0.1 M solutions of acidic counter-ions in MeOH were prepared. 100 μl of drug and 30 μl (one equivalent) of counter-ion solution were added in duplicate to 96 well plates. The plates were covered and placed at 25° C. and 50° C. for several days until the solvent had evaporated. The wells were analyzed using light microscopy for the presence of crystalline, birefringent material. The presence of crystalline material is a pointer towards successful salt formation.

Following initial analysis, further solvents were added and, after evaporation, the samples were analyzed by light microscopy. Subsequent solvents used included ethyl acetate, acetonitrile, water, dichloromethane:petroleum ether (50:50), ethanol, toluene, iso-propyl acetate, propan-2-ol and acetone.

The study highlighted HCl and HBr as potential crystalline salts. The study was repeated using a batch of ultra-pure Compound A1 free base. The study methodology outlined previously was repeated this time using both one and two equivalents of counter-ion. Using ultra-pure starting material, the following counter-ions yielded useful salts: napsylate, edisylate, adipate, ascorbate, besylate, sulphate, tosylate, esylate, fumarate, and mesylate.

Hits were scaled up using the following general methodology: 50 mg of drug was dissolved in 3 ml of isopropyl acetate. This solution was added to a 1.1 molar equivalent or 2.1 molar equivalent solution of acid in 10 ml of isopropyl acetate. The resultant mixture was heated and stirred for 10 min, then allowed to cool. In the case of the fumarate, esylate, besylate, sulphate and tosylate a solid precipitated out of solution, this was filtered, washed with cold cyclohexane and allowed to dry. In the case of napsylate, edisylate, adipate and ascorbate an oil was produced.

Salt Analysis. The fumarate, esylate, besylate, sulphate and tosylate material prepared on a 50 mg scale were analyzed by XRPD to assess crystallinity. The sulphate, besylate and tosylate scale-ups were amorphous by XRPD. The esylate scale-up material was amorphous with some crystallinity by XRPD, the fumarate scale-up material scale-up material was crystalline by XRPD. The HCl, HBR, mesylate, esylate, and fumarate were further scaled up (1-g scale).

The HCl, HBR, mesylate, esylate, and fumarate salts of Compound A1 contained crystalline material as observed by XRPD. The stoichiometry of the fumarate salt was determined to be 1.5 moles fumarate per mole of Compound A1, i.e., a sesquifumarate salt.

DVS data were obtained for the HCl, HBR, mesylate, esylate and fumarate salts of Compound A1. The HBr, HCl and fumarate salts absorbed moisture in a roughly linear fashion between 0 and 95% relative humidity (RH); no hysteresis was observed. The HBr salt absorbed about 9% moisture at 80% RH and about 11% moisture at 95% RH. The HCl salt absorbed about 11% moisture at 80% RH and about 12% moisture at 95% RH. The fumarate salt absorbed about 3.5% moisture at 80% RH and about 4.0% moisture at 95% RH. Moisture uptake for the HBr, HCl and fumarate salts was linear and reversible.

The mesylate and esylate salts of Compound A1 showed similar DVS profiles to each other, absorbing relatively low levels of moisture at low RH, then deliquescing at high RH; no hysteresis was observed. The mesylate salt absorbed about 2% moisture up to 70% RH and about 20% moisture at 95% RH, when deliquescence occurred. The esylate salt absorbed about 3% moisture up to 60% RH and about 20% moisture at 95% RH, when deliquescence occurred.

### Solid Forms Comprising the Mono-HCl Salt of Compound A1

Solid forms comprising a mono-HCl salt of Compound A1 were prepared. Details regarding the preparation and characterization of such solid forms are provided herein.

### Form I of the Mono-HCl Salt of Compound A1

### Example Preparation 1

Dissolved 1 g of Compound A1 (92.7% purity, 0.927 g, 1.95 mmol) in ethanol (7 mL). The solution was placed on a hot plate set at 55° C. Added, with stirring, 1.25 M hydrogen chloride in 2-propanol (1.56 mL, 1.95 mmol). A solid immediately came out of solution. The mixture was stirred on the hot plate at 55° C. for one hour. The mixture was then allowed to cool to and stir at ambient temperature for four hours. The solid was collected by suction filtration, washed with cold ethanol (10 mL) and dried under vacuum at ambient temperature to give Form I of the mono-HCl salt of Compound A1 (960 mg) as a white solid.

### Example Preparation 2

Dissolved 1.01 g of Compound A1 (92.7% purity, 0.936 g, 1.97 mmol) in ethanol (10 mL). The solution was placed on a hot plate set at 75° C. Added, with stirring, 1.25 M hydrogen chloride in 2-propanol (1.58 mL, 1.97 mmol). A solid immediately came out of solution. Additional ethanol (5 mL) was added to facilitate stirring of the mixture. The mixture was stirred on the hot plate at 75° C. for thirty minutes. The mixture was then allowed to cool to and stir at ambient temperature for three hours and then in an ice bath for one hour. The solid was collected by suction filtration, washed with cold ethanol (10 mL) and dried under vacuum at ambient temperature to give Form I of the mono-HCl salt of Compound A1 (954 mg) as a white solid.

### Example Preparation 3

The sesquifumarate salt of Compound A1 was slurried in isopropyl acetate and water. Solid sodium carbonate was charged in 4 portions. Complete dissolution was achieved. Following phase separation, the upper organic phase was re-washed with water. The resulting organic phase was concentrated under reduced pressure. The oil obtained was redissolved in isopropyl acetate and heated to 60° C. before a solution of HCl in IPA was added. Precipitation was seen on addition of the HCl. The resulting slurry was cooled to ambient, the solid was collected by filtration and washed with isopropylacetate before being dried in the oven under vacuum at 40° C.

### Example Preparation 4

The sesquifumarate salt of Compound A1 was slurried in ethyl acetate and water. A solution of potassium hydroxide was added slowly, maintaining the pH below 9 and complete dissolution was achieved. Following phase separation, the upper organic phase was re-washed with weak alkaline solution and then water. The resulting organic phase was dried over sodium sulphate before being concentrated under a reduced pressure. This provided the free base as a foam that could be scraped off and characterized.

The solvents tert-butylmethyl ether, tetrahydrofuran, isopropyl acetate and ethanol were investigated for their suitability for salt formation. The solvent selection was carried out in a Radley's carousel. The free base was dissolved in the solvent and then hydrochloric acid in IPA was slowly added. Generally the HCl salt precipitated out of all of the solvents. However, ethanol and THF both provided white material (the liquors were dark) whereas the solid isolated from MTBE and IPAC was off-white (the liquors were much clearer). The yield for all of the salt preparations was greater than 80% and the purity ranged from 99.1% by HPLC to 99.9% by HPLC. All of the material had some (4-6%) solvent associated with it.

### Example Preparation 5

Compound A1 free base was dissolved in ethanol and the solution was warmed to 70° C. A solution of hydrogen chloride in IPA was added slowly. On a 20 g scale, precipitation was not apparent until the batch had been cooled to 40° C. and seeded. However, on scale up to 130 g, the batch did precipitate at 60° C. without seeding. The slurry became very thick, thus further solvent was added to the batch to help mobilize it. The product was isolated as a white solid. The crystalline mono-HCl salt of Compound A1 was isolated in high purity. Analysis: NMR strength (as free base)=87% w/w; Rel subs: total imps=0.06 Area % (No decomposition observed from free base); Chloride content=7.5% w/w; Water content=4.3% w/w; Residual solvent: EtOH=5.0% w/w; IPA=0.05% w/w.

### Example Preparation 6

Compound A1 free base (137 g; 1.00 equiv; 288.65 mmoles) was charged to a 2 L jacketed vessel equipped with a condensor, an overhead stirrer, and a thermometer probe. Ethanol (30.59 moles; 1.78 L; 1.41 kg) was charged to the vessel and the contents were stirred. The agitator was set at 350 rpm. Complete dissolution was achieved.•The contents of the vessel were warmed to 70° C. Hydrogen chloride (360.81 mmoles; 72.37 mL; 65.78 g) was charged to a dropping funnel. The hydrochloric acid was slowly added to the solution in the vessel over approximately 40 mins. Partway through the addition, precipitation was seen. The reaction mixture became a thick slurry. Following complete addition of the hydrochloric acid, the reaction was stirred at 70° C. for 1 hr before being cooled to ambient over 2 hours. Ethanol (4.71 moles; 274.00 mL; 216.82 g) was charged to the vessel and transferred to the cake as a displacement wash.•The cake was deliquored.•Ethanol (4.71 moles; 274.00 mL; 216.82 g) was charged to the vessel and transferred to the cake as a displacement wash. The cake was deliquored. The product was transferred to the oven and dried under reduced pressure at 40° C. to provide 131.06 g of white solid, which was crystalline Form I of the mono-HCl salt of Compound A1. (Analysis of product: NMR strength 87%; Rel subs 0.06% area; Cl 7.5% w/w; water 4.3% w/w; EtOH 5.0% w/w.) The Form I mono-HCl salt was synthesized in good yield and high purity; the starting free base material was 98.6% purity by HPLC, and the output mono-HCl salt material contained only 0.06% impurities.

Solubility and Dissolution Rate Data. The solubility and dissolution rate of Form I of the mono-HCl salt of Compound A1 were measured in various media. In water, the solubility >41 mgA/ml (pH=4.2). In 0.1M phosphate acid, the solubility was >40 mgA/ml (pH=3.3). Additional solubility and dissolution rate data is provided in Table 1 and Table 2.

TABLE 1
Solubility of Form I of the Compound A1 Mono-HCl Salt in Simulated
Biological Media (Mean ± SD, n = 2)

| | Compound A1 mono-HCl Salt (mg/mL) | | | |
|---|---|---|---|---|
| Medium*/ | | pH of the | | pH of the |
| Initial pH | 3 hr | mixture | 24 hr | mixture |
| SGF, pH 1.3 | 23.1 ± 0.12 | 2.82 | 21.9 ± 0.23 | 2.81 |
| FaSSIF, pH 6.51 | 1.72 ± 0.23 | 6.53 | 0.83 ± 0.00 | 6.45 |
| FeSSIF, pH 5.03 | 3.40 ± 0.01 | 5.14 | 3.06 ± 0.06 | 5.11 |
| 0.1M phosphate | 0.29 ± 0.07 | 7.59 | 0.10 ± 0.01 | 7.51 |
| buffer, pH 7.5 | | | | |

*As used herein, SGF = Simulated gastric fluid; FaSSIF = Fasted-state simulated intestinal fluid; FeSSIF = Fed-state simulated intestinal fluid.

TABLE 2
Intrinsic Dissolution Rate of Form I of the Compound A1 Mono-HCl Salt
in Simulated Biological Media (Mean ± SD, n = 2)

Compound A1 mono-HCl Salt

| Medium/ | | IDR |
|---|---|---|
| Initial pH | Final pH | (μg/min/cm²) |
| SGF, pH 1.3 | N/A | Too soluble to |
| | | be determined |
| FaSSIF, pH 6.51 | 6.48 | 48.4 ± 7.12 |
| FeSSIF, pH 5.03 | 4.99 | 91.6 ± 5.48 |

Chemical Stability. The chemical stability of Form I of the mono-HCl salt of Compound A1 was assessed by stressing the material at various temperature/relative humidity/light conditions, then analyzing by HPLC. Results are presented in Table 3.

TABLE 3
Chemical Stability Data for Form I
of the Mono-HCl Salt of Compound A1

| | | | % Purity | % Purity | % Change |
|---|---|---|---|---|---|
| | Time point | | (reference | (after | from |
| Condition | (days) | | material) | challenge) | reference |
| Reference | 0 | | 100 | 99.8 | 0.0 |
| 25° C./ | 7 | | 103.2 | 99.8 | 0.0 |
| 60% RH | | | | | |
| 40° C./ | 7 | | 102.1 | 99.8 | 0.0 |
| 75% RH | | | | | |
| 80° C. | 7 | | 101.4 | 99.8 | 0.0 |
| Ultraviolet Light, | ICH | (1 day) | 98.0 | 99.6 | −0.2 |
| 25° C./ | | | | | |
| 60% RH* | | | | | |
| Ultraviolet light, | 2x ICH | (2 days) | 95.0 | 99.2 | −0.6 |
| 25° C./ | | | | | |
| 60% RH* | | | | | |
| Ultraviolet light, | 3x ICH | (3 days) | 94.7 | 99.0 | −0.8 |
| 25° C./ | | | | | |
| 60% RH* | | | | | |
| White light, | ICH | (7 days) | 95.6 | 98.5 | −1.3 |
| 25° C./60%* | | | | | |

*Light samples were stored open. Unlike other samples which were weighed prior to storage, the light samples were weighed for assay after storage.

TABLE 4
Chemical Stability Data for Two Samples of
Form I of the Mono-HCl Salt of Compound A1

| | % Purity | % Purity | % Purity | % Purity | % Purity | % Purity |
|---|---|---|---|---|---|---|
| | after | after | after | after | after | after |
| Initial | 40° C./75% | 40° C./75% | 60° C./80% | 60° C./80% | 80° C., | 80° C., |
| Purity* | RH, 2 wks | RH, 4 wks | RH, 2 wks | RH, 4 wks | 2 wks | 4 wks |
| 98.7% | 98.8 | 98.8 | 97.6 | 95.3 | 96.9 | 95.4 |
| 97.0% | 97.3 | 97.2 | 90.4 | 89.0 | 94.1 | 92.3 |

*All purity values assessed by HPLC area %.

Single Crystal X-ray Diffraction. The crystal structure of the Form I crystal form of the mono-HCl salt of Compound A1 was solved by single-crystal X-ray diffraction according to the procedure described below.

Sample Preparation. The mono-HCl salt of Compound A1 was dissolved in isopropanol and water and left at ambient temperature to evaporate. Single crystals were obtained prior to complete evaporation.

Data Collection. A single crystal of empirical formula C₃₁H₄₉ClFN₄O_{2.5}, having approximate dimensions of 0.3117×0.1670×0.0166 mm, was isolated and mounted on a glass fiber. Preliminary examination and data collection were performed with Mo K_{α} radiation (λ=0.71073 Å) on an OXFORD Xcalibur3 diffractometer. The data were collected at a temperature of about 173 K. The crystal structure was solved and refined with the SHELXTL package, as known in the art.

Simulated X-ray Powder Diffraction (XRPD) Pattern. A simulated XRPD pattern was generated for Cu radiation and the atomic coordinates, space group, and unit cell parameters from the single crystal data collected at about 173 K.

Asymmetric Unit and Packing Diagrams. Asymmetric unit diagrams and packing diagrams were prepared as known in the art.

Results from Single Crystal X-ray Analysis on Form I of the mono-HCl Salt of Compound A1. The tetragonal approximate cell parameters and approximate calculated volume were determined to be: a=18.4332(15) Å, b=18.4332(15) Å, c=18.671(4) Å, α=90°, β=90°, γ=90°, V=6344.2(14) Å³. The formula weight was 572.19 g/mol with Z=8 and a calculated density of 1.198 Mg/m³. The space group was determined to be P4(3)2(1)2 (no. 96). Crystal data and crystallographic data collection parameters are summarized in Table 5.

The absolute configuration of Compound A1 was established by using the anomalous dispersions of the Cl atom, and the molecule was found to be a (R)-isomer. The mono-HCl salt of Compound A1 comprised 1 molar equivalent of isopropanol and 1.5 molar equivalents of water per mole of the mono-HCl salt. A drawing of the asymmetric unit of Form I of the mono-HCl salt of Compound A1, as isolated from isopropanol and water, is shown in FIG. 9. The single crystal structure is in agreement with the Compound A1 chemical structure, depicted above as structure (I).

In the crystal, the proton was transferred to one of the nitrogen atoms in the piperazine ring and formed a hydrogen bond with the chloride anion (N . . . Cl 3.097(5) Å). The water molecules formed hydrogen bonds with chlorides and IPA molecules, and a channel-like structure with large cavities in the crystal lattice was generated.

The simulated XRPD pattern (shown in FIG. 5), calculated from atomic coordinates of the crystal structure at −100° C., matches with the experimental XRPD of the bulk material, suggesting that the crystal for the single crystal analysis is representative to the bulk material. Slight shifts in peak location between the simulated XRPD pattern and experimental XRPD patterns may relate to the fact that the experimental powder pattern was collected at ambient temperature, while the single crystal data was collected sub-ambient temperature.

TABLE 5
Crystal Data and Refinement for a crystal of Form I of the Mono-
HCl Salt of Compound A1, Isolated from Isopropanol and Water

| Empirical formula | C31 H49 Cl F N4 O2.50 | |
|---|---|---|
| Formula weight | 572.19 | |
| Temperature | 173(2) K | |
| Wavelength | 0.71073 Å | |
| Crystal system | Tetragonal | |
| Space group | P4(3)2(1)2 | |
| Unit cell dimensions | a = 18.4332(15) Å | α = 90°. |
| | b = 18.4332(15) Å | β = 90°. |
| | c = 18.671(4) Å | γ = 90°. |

| | |
|---|---|
| Volume | 6344.2(14) Å³ |
| Z | 8 |
| Density (calculated) | 1.198 Mg/m³ |
| Absorption coefficient | 0.161 mm⁻¹ |
| F(000) | 2472 |
| Crystal size | 0.3117 × 0.1670 × 0.0166 mm³ |
| Theta range for data collection | 3.95 to 26.13°. |
| Index ranges | −22 <= h <= 22, −21 <= |
| | k <= 22, −23 <= 1 <= 22 |
| Reflections collected | 45910 |
| Independent reflections | 6308 [R(int) = 0.0646] |
| Completeness to theta = 26.13° | 99.4% |
| Absorption correction | Analytical |
| Max. and min. transmission | 0.983 and 0.924 |
| Refinement method | Full-matrix least-squares on F² |
| Data/restraints/parameters | 6308/6/349 |
| Goodness-of-fit on F² | 1.053 |
| Final R indices [I > 2sigma(I)] | R1 = 0.0898, wR2 = 0.2336 |
| R indices (all data) | R1 = 0.1323, wR2 = 0.2807 |
| Absolute structure parameter | −0.04(15) |
| Largest diff. peak and hole | 0.820 and −0.409 e.Å⁻³ |

### Amorphous Form of the Mono-HCl Salt of Compound A1

Vacuum-Drying Procedure. The vacuum-dried amorphous form was prepared by drying solvated crystalline HCl material at 80° C. The resulting solid was amorphous as analyzed by XRPD. Analysis: NMR strength=94% w/w; Rel subs: total imps=0.71 Area % (decomposition is observed during the vacuum drying at 80° C.); Chloride content=6.7% w/w; Water content=1.2% w/w; Residual solvent: None detected.

Solubility Testing for Spray Drying. To spray dry the material, it was desirable to have >10% w/v solubility in a solvent that has a boiling point near or below the glass transition temperature. With the Compound A1 mono-HCl salt, the glass transition temperature was affected by the residual solvent and moisture. The solubility of Compound A1 mono-HCl salt was tested in solvents including tetrahydrofuran, methanol, acetone and methyl tert-butylether. Methanol provided optimum solubility for spray drying.

Spray-Drying Procedure 1. A trial was conducted to test whether spray drying was a viable manufacturing technique for preparation of the amorphous form of the mono-HCl salt of Compound A1. A Buchi spray drier was used. Key success factors for this trial included: (1) Successful processing with limited or no “glassing” of the product in the spray drying equipment and reasonable yield (i.e. >5-10%); (2) Fully amorphous product by XRPD and optical microscopy; and (3) Product of acceptable chemical stability with respect to impurities, salt stoichiometry and residual solvents. Data on the physical properties of the material were also measured including glass transition temperature and hygroscopicity.

Spray drying was carried out at a 5-g input scale using a 10% w/v solution in methanol; the solution was warmed to 35° C. to dissolve all solids. Outlet temperatures were between 85° C. and 71° C. during the trial.

No operational issues were encountered and no significant glassing took place. The yield obtained was 40% (2 g product). The product was split into two portions—cyclone recovery and collection vessel. Overnight secondary-drying was performed on all materials.

Spray-Drying Procedure 2. A second trial was carried out. The procedure and equipment used were the same as those described above for Spray-Drying Procedure 1, with the exception that a larger quantity of input material was used (8.63 g).

Outlet temperatures in this trial were slightly lower in the range 79° C. to 71° C. No significant glassing took place. The yield obtained was 41% (3.57 g). This trial was split into two parts as the exhaust filter on the spray dryer blocked midway through the trial. The product was therefore split into four portions: cyclone recovery and collection vessel from each of the two parts. The filter, cyclone and product receiver were all cleaned in between the two parts of the trial. Overnight secondary-drying was performed on all materials.

A comparison of Form I and the amorphous form of the mono-HCl salt of Compound A1 was performed. The Form I material had an NMR strength of 95.3% w/w; solvent content of 0.5% IPAC; a total impurity level of 1.12 area % by HPLC; and a chloride content of 6.3% w/w corrected for strength. The spray-dried amorphous material had an NMR strength of 91.8% w/w; no measured solvent content; a total impurity level of 1.61 area % by HPLC; and a chloride content of 5.7% w/w corrected for strength.

Spray-Drying Procedure 3. Two spray-drying tests were performed at larger scales using industry-standard spray-drying equipment and parameters. Test 1 was performed starting with about 10 g of the mono-HCl salt of Compound A1 in about 79 g of methanol. Test 2 was performed starting with about 90 g of the mono-HCl salt of Compound A1 in about 712 g of methanol. Heating was required to fully dissolve the materials. After heating, the solution was formed and the feed solution was filtered. The filtrate was ready to be spray dried and was not heated or agitated during the test. Each test was composed of two parts; the first part comprised spray drying, and the second part comprised post-drying the product.

Test 1 resulted in a yield of 39% (excluding filter recovery). In Test 2, the operating parameters were optimized (increased feed rate and decreased atomizing gas rate) to increase particle size and yield. Test 2 resulted in a yield of about 80%.

After the spray drying tests, the two products were collected and vacuum dried in an oven at 40° C. for about 12 h. Residual solvent analysis of the undried Test 2 material showed 1.6% methanol (the filter recovery product showed 1% methanol). Initial overnight drying of this material reduced the methanol content to 1.3%.

Additional drying was performed. The Test 2 material was dried overnight (about 20 hours) in a vacuum oven (about 400 mbar) fitted with an argon purge and containing desiccant at a temperature of 40° C. After this overnight drying session, the Test 2 sample was reduced to 0.77% methanol w/w. The material was dried for a further 24 hours in a vacuum oven (about 400 mbar) fitted with an argon purge and containing desiccant at a temperature of 40° C. After this 24-hour drying session, the Test 2 sample was reduced to 0.51% methanol w/w.

Chemical analysis. Analysis was performed on various recovered samples, including wet samples, samples dried once, and secondary-dried samples. Samples were approximately 99.9% pure by HPLC. Solvent content was determined by gas chromatography. For ethanol, solvent content ranged from none detected to 0.9%. For isopropanol, solvent content ranged from none detected to 0.01%. For methanol, solvent content ranged from 0.05% to 1.57%. NMR strength ranged from 95% w/w to 99% w/w. Water content by Karl Fisher ranged from 0.82% w/w to 1.11% w/w. Chloride content values analyzed by IC ranged were 6.5%, 6.6%, 6.9%, and 7.5%. Chloride content was close to the expected value of 6.9% for the monohydrochloride salt.

Hygroscopicity. Dynamic Vapor Sorption (DVS) measurements were performed on the amorphous form of the mono-HCl salt of Compound A1. Studies on the amorphous mono-HCl salt prepared by spray drying methods showed that the material picks up about 14% weight of moisture at 90% RH. The material deliquesced in high humidity and became a glass-like material after losing water at 0% RH.

The amorphous material was placed into preset RH conditions (50% and 75% RH) for 24 hours to check dynamic moisture absorption as well as deliquescence. When the material was dried at 0% RH, it had about 2% weight loss. When placed at 50% RH, it quickly absorbed about 4% of water within the first half hour and extra 1.5% water after 24 hours. No crystallization was observed by XRPD after 50% DVS study, and the particle morphology did not change as compared to the pre-DVS starting material, as observed by polarized light microscopy (PLM).

The material quickly absorbed about 5.5% of water when directly placed at 70% RH (starting material contained about 2% of water). No crystallization was observed by XRPD after 70% RH DVS study, and the particle morphology did not change as compared to the pre-DVS starting material, as observed by PLM.

Physical Stability. Physical stability measurements were performed on the amorphous form of the mono-HCl salt of Compound A1. The amorphous HCl salt prepared by spray drying was set down under two conditions (40° C./75% RH and 25° C./60% RH) for physical stability studies. After the material was exposed to 40° C./75% RH (open) for 2 weeks, the material deliquesced and became brown in color. However, after 17 days at 25° C./60% RH (open), the material was physically stable. The particles maintained their morphology and did not deliquesce. The glass transition temperature (Tg) of the resulting material decreased to 52° C. (the initial material had a Tg of 80° C.), and a Karl-Fisher study indicated that the material contained 6.1% water (the initial material of contained about 2.8% water).

Glass Transition Temperature (Tg) and Moisture Impact. Tg values of amorphous mono-HCl salt were measured by the modulated-DSC (mDSC) method and found to be between about 68° C. and about 80° C. (e.g., 74.9° C., 68.0° C., 80.1° C., 73.2° C.). The differences in Tg values were likely related to the different water (or solvent) contents in these materials.

To investigate the impact of water on the amorphous mono-HCl salt, the material was stored at various RH conditions controlled by saturated salt solutions. Each material was checked by PLM (morphology), KF (water content), and mDSC (Tg value) after 3 days. There were no visible changes for materials at 23% RH, 43% RH, 54% RH, or 76% RH. Materials became yellow at 85% RH and deliquesced at 97% RH. PLM showed that the morphology of materials at 76% RH is similar to the original material, while the material at 85% RH started to agglomerate.

Results are summarized in the following table. Moisture uptake significantly increased near linearly when the RH increased, and the Tg value sharply decreased from 127° C. (dry condition) to 33° C. (9.5% of water content in the material when place into 85% RH).

TABLE 6
Moisture Impact of Tg for the Amorphous
Mono-HCl Salt of Compound A1

| | Saturated | | | |
|---|---|---|---|---|
| RH | Solution | Appearance | Water content | Tg |
| 23.1% | KOAc | No change | 3.2% | 72.1° C. |
| 43.3% | K₂CO₃ | No change | 5.0% | 63.2° C. |
| 54.4% | Mg(NO₃)₂ | No change | 6.2% | 56.5° C. |
| 75.5% | NaCl | No change | 8.2% | 44.2° C. |
| 85.1% | KCl | Yellow | 9.5% | 33.0° C. |
| 97.3% | K₂SO₄ | Deliquesced | 14.7% | Not determined |

Chemical stability data. The amorphous mono-HCl salt of Compound A1 was assessed for chemical stability under specific conditions for specific time points. Data is presented in Table 7.

TABLE 7
Chemical Stability Data for the Amorphous Mono-HCl Salt of Compound A1

| | | | | | Purity after |
|---|---|---|---|---|---|
| | Purity after | Purity after | Purity after | Purity after | Photostability |
| Initial | 60° C./80% RH | 60° C./80% RH | 80° C., | 80° C., | 3X ICH |
| Purity* | 2 weeks | 4 weeks | 2 weeks | 4 weeks | conditions |
| 98.8% | 94.4% | 94.5% | 98.7% | 98.6% | 82.3% |

*All purity values assessed by HPLC area %.

Formulations and excipient compatibility data. The amorphous mono-HCl salt of Compound A1, prepared by vacuum drying, was assessed for excipient compatibility under specific conditions for specific time periods. The amorphous vacuum-dried Compound A1 mono-HCl salt was combined with particular pharmaceutical excipients used in immediate release formulations in two separate formulations. The two formulations were studied at various accelerated/stressing conditions, up to 12 weeks. The substance demonstrated acceptable stability when incorporated into the example formulations at the conditions and intervals studied.

“Formulation 1” comprised the following ingredients: Amorphous Compound A1 mono-HCl (5 mg); Avicel® PH 113 (15 mg); Perlitol® SD200 (64 mg); L-HPC LH-11 (15 mg); magnesium stearate (1 mg). “Formulation 2” comprised the following ingredients: Amorphous Compound A1 mono-HCl salt (5 mg); Avicel® PH 113 (64 mg); A-TAB®, (Rhodia) (25 mg); PolyPlasdone XL 10 (5 mg); magnesium stearate (1 mg).

Excipient compatibility data corresponding to Formulation 1 and Formulation 2 is presented in Table 8.

TABLE 8
Excipient Compatibility Data for the Amorphous
Mono-HCl Salt of Compound A1

Potency of amorphous mono-HCl salt of
Compound A1*

| Condition | Formulation 1 | Formulation 2 |
|---|---|---|
| Initial | 97.8 | 97.8 |
| 25° C., 2 weeks | 97.8 | 97.8 |
| 25° C., 4 weeks | 98.6 | 98.5 |
| 40° C., 2 weeks | 98.0 | 97.5 |
| 40° C., 4 weeks | 98.7 | 97.9 |
| 40° C., 12 weeks | 97.8 | 97.3 |
| 40° C., 75% RH, | 97.4 | 96.7 |
| 2 weeks | | |
| 40° C., 75° RH, | 96.3 | 96.4 |
| 4 weeks | | |

*All purity values assessed by HPLC area %

### Amorphous Form Comprising the Tri-HCl Salt of Compound A1

An amorphous solid form comprising the tri-HCl salt of Compound A1 was prepared. Details regarding its preparation and characterization are provided herein.

### Example Preparation 1

250 mg of Compound A1 free base was taken up in 5-6 N HCl in isopropanol, and warmed to 40° C. to give a clear orange solution. Addition of MTBE as an antisolvent caused precipitation of a white solid that was collected by filtration. Material was extremely hygroscopic and turned to gel even under constant stream of nitrogen.

### Example Preparation 2

7.0 g of Compound A1 free base was taken up in 5-6 N HCl in isopropanol (250 ml), and warmed to 40° C. at which point almost all material was in solution. The solution was transferred to a clean flask and MTBE added as an anti-solvent. White precipitate was collected by filtration under constant inert atmosphere. A small quantity of material (about 250 mg) was isolated quickly and placed under vacuum immediately to allow analysis for chloride content. Chloride determination by titration against silver nitrate showed an HCl strength of 295 mol %, indicating presence of the tri-HCl salt of Compound A1. Further evidence that the material was the tri-HCl salt was provided by strength by NMR experiment against maleic acid.

### Example Preparation 3

The remainder of the material from Example Preparation 2 proved too hygroscopic to isolate by filtration (even when employing a nitrogen filled glove-bag) and was lyophilized to give 3.4 g of an air-stable solid. This lyophilized amorphous material was characterized by NMR spectroscopy and found to be 97% pure tri-HCl salt of Compound A1. The stoichiometry was assessed via chloride determination, and was confirmed to be 300 mol % HCl. The water content was measured and determined to be 3.0% by weight. As observed by PLM, the material appeared as a glass.

Chemical Stability Data. The amorphous tri-HCl salt of Compound A1 was assessed for chemical stability under specific conditions for specific time points. Data is presented in Table 9.

TABLE 9
Chemical Stability Data for the Amorphous
Tri-HCl Salt of Compound A1

| | Purity after | Purity after | Purity after | Purity after |
|---|---|---|---|---|
| Initial | 60° C./80% | 60° C./80% | 80° C., | 80° C., |
| Purity* | RH 2 weeks | RH 4 weeks | 2 weeks | 4 weeks |
| 97.7% | 93.5% | 89.7% | 93.1% | 91.1% |

*All purity values assessed by HPLC area %.

### Solid Form Comprising a Sulfate Salt of Compound A1

An amorphous solid form comprising a sulfate salt of Compound A1 was prepared. Details regarding its preparation and characterization are provided herein.

### Example Preparation 1

Dissolved 1 g of Compound A1 free base (92.7% purity, 0.927 g, 1.95 mmol) in ethyl acetate (10 mL). Added, with stirring, 1 M H₂SO₄ in 2-propanol (1.95 mL, 1.95 mmol). A gelatinous mass formed. Placed the flask on a hotplate at 80° C. and stirred vigorously. An additional 5 mL of ethyl acetate was added to the flask. The mass rapidly turned to a solid. The solid was stirred at on the 80° C. hot plate for 30 minutes and then stirred at ambient temperature for three hours. The solid was collected by suction filtration, washed with ethyl acetate (15 mL), and dried under vacuum overnight at ambient temperature to give an amorphous solid form of a sulfate salt of Compound A1 (918 mg) as an off-white solid.

### Example Preparation 2

50 mg of Compound A1 was dissolved in 3 ml of isopropyl acetate. This was added to a 1.1 molar equivalent solution of acid in 10 ml of isopropyl acetate. The resultant mixture was heated and stirred for 10 minutes then allowed to cool. A solid precipitated out of solution; this was filtered, washed with cold cyclohexane, and allowed to dry. This solid was characterized as an amorphous solid form of the sulfate salt of Compound A1.

Characterization. Stability data for the amorphous form comprising the sulfate salt of Compound A1 is provided in Table 10. Other characterization data for the amorphous form comprising the sulfate salt of Compound A1 is provided elsewhere herein.

TABLE 10
Chemical Stability Data for Two Samples of the Sulfate Salt of Compound A1

| | % Purity | % Purity | % Purity | % Purity | % Purity | % Purity |
|---|---|---|---|---|---|---|
| | after | after | after | after | after | after |
| Initial | 40° C./75% | 40° C./75% | 60° C./80% | 60° C./80% | 80° C., | 80° C., |
| Purity* | RH, 2 wks | RH, 4 wks | RH, 2 wks | RH, 4 wks | 2 wks | 4 wks |
| 93.3% | 92.6 | 91.2 | 74.1 | 62.3 | 78.5 | 67.1 |
| 94.7% | 88.7 | 87.3 | 80.5 | 80.6 | 88.7 | 85.7 |

*All purity values assessed by HPLC area %.

### Solid Form Comprising a Phosphate Salt of Compound A1

An amorphous solid form comprising a phosphate salt of Compound A1 was prepared. Details regarding its preparation and characterization are provided herein.

### Example Preparation 1

Dissolved 0.52 g of Compound A1 free base (92.7% purity, 0.48 g, 1.02 mmol) in cyclopentylmethyl ether (2 mL). Added, with stirring, 2M H₃PO₄ in ethanol (0.51 mL, 1.02 mmol). A soft semi-solid came out of solution. The solution was heated to boiling and an additional 0.7 mL of ethanol was added to dissolve the semi-solid. The solution was allowed to cool to ambient temperature and then placed in the freezer overnight.

The next morning, there was no crystallization. The solution was concentrated under an argon stream and then under vacuum. To the resulting residue was added ethyl acetate (7 mL). The residue was stirred vigorously on a hot plate at 90° C. The residue broke up into a fine solid. After about 40 minutes at 90° C., the mixture was allowed to cool to and stir at ambient temperature for two hours. The solid was collected by suction filtration, washed with cold ethyl acetate (5 mL) and dried under vacuum at ambient temperature to give a phosphate salt of Compound A1 (384 mg) as an off-white solid.

Characterization. Stability data for the amorphous form comprising the phosphate salt of Compound A1 is provided in Table 11. Other characterization data for the amorphous form comprising the phosphate salt of Compound A1 is provided herein.

TABLE 11
Chemical Stability Data for the Phosphate Salt of Compound A1

| | % Purity | % Purity | % Purity | % Purity | % Purity | % Purity |
|---|---|---|---|---|---|---|
| | after | after | after | after | after | after |
| Initial | 40° C./75% | 40° C./75% | 60° C./80% | 60° C./80% | 80° C., | 80° C., |
| Purity* | RH, 2 wks | RH, 4 wks | RH, 2 wks | RH, 4 wks | 2 wks | 4 wks |
| 91.2% | 88.6 | 87.7 | 81.4 | 78.4 | 86.6 | 83.4 |

*All purity values assessed by HPLC area %.

### Solid Form Comprising the Sesquifumarate Salt of Compound A1

A Form I crystal form comprising the sesquifumarate salt of Compound A1 was prepared. Details regarding its preparation and characterization are provided herein.

### Example Preparation 1

To a solution of fumaric acid (36.9 g, 318 mmol) in EtOH (700 mL) at 35° C. was added a solution of Compound A1 free base (92.3 g, 194 mmol) in EtOH (500 mL), in a controlled manner. The mixing of the two solutions led to crystallization. The slurry was cooled to 20° C. and stirred at this temperature for a further 16 h. The product was collected by filtration and washed with EtOH (100 mL), before being dried under reduced pressure at 60° C. to provide 114.5 g of Form I of the sesquifumarate salt of Compound A1.

Purity (HPLC): >98%; Optical Purity (chiral HPLC): >98%; ¹H NMR (d⁶-DMSO) δ_{H} 1.06 (br, 6H), 2.30 (br, 2H), 2.36 (br, 2H), 2.47 (br, 4H), 3.16 (br, 2H), 3.37 (br, 2H), 3.54 (s, 2H), 4.11 (1H, s), 6.37 (dd, J=1.3 and 7.9 Hz, 1H), 6.55 (d, J=7.6 Hz, 1H), 6.62 (t, J=1.8 Hz, 1H), 6.62 (s, 2H), 6.91 (t, J=7.7 Hz, 1H), 7.13 (t, J=8.8 Hz, J_{HF}=8.8 Hz, 2H), 7.25 (d, J=8.1 Hz, 2H), 7.32 (dd, J=8.4 Hz, J_{HF}=5.8 Hz, 2H), 7.42 (d, J=8.1 Hz, 2H). IR (cm⁻¹) 2973 (NH), 1725 (C═O), 1708 (C═C), 1613 (C═O), 1563 (C═C). Found: C, 62.15; H, 6.35; N, 8.20. 2×C₂₉H₃₅FN₄O with 3×C₄H₄O₄ and 4.5% H₂O requires C, 61.88; H, 6.58; and N, 8.25%.

### Example Preparation 2

50 mg of Compound A1 free base was dissolved in 3 ml of isopropyl acetate. This solution was added to a 2.1 molar equivalent solution of fumaric acid in 10 ml of isopropyl acetate. The resultant mixture was heated and stirred for 10 minutes then allowed to cool. A solid precipitated out of solution, this was filtered, washed with cold cyclohexane and allowed to dry.

Solubility and Dissolution Rate Data. The dissolution rate and equilibrium solubility of Form I of the sesquifumarate salt of Compound A1 in various buffers and solvents were measured. Results are presented in Table 12, Table 13, and Table 14.

TABLE 12
Equilibrium Solubility of Compound
A1 Sesquifumarate Salt at 25° C.

| Medium | Equilibrium solubility (mg/mL) |
|---|---|
| Pure water | 8.7 |
| 0.1N HCl | 21.6 |
| pH 4.5, Citrate 0.1M | 15.6 |
| pH 7.8, Phosphate 0.1M | 0.16 |
| 0.1N NaOH | 0.092 |

TABLE 13
Solubility of Compound A1 Sesquifumarate Salt in Simulated
Biological Media (Mean ± SD, n = 2)

| | Sesquifumarate Salt (mg/mL) | | | |
|---|---|---|---|---|
| Medium/ | | pH of the | | pH of the |
| Initial pH | 3 hr | mixture | 24 hr | mixture |
| SGF, pH 1.3 | 17.4 ± 0.28 | 2.83 | 17.1 ± 0.14 | 2.75 |
| FaSSIF, pH 6.51 | 4.40 ± 0.13 | 5.19 | 4.13 ± 0.10 | 5.27 |
| FeSSIF, pH 5.03 | 2.54 ± 0.24 | 4.95 | 2.66 ± 0.13 | 4.90 |
| 0.1M phosphate | 0.18 ± 0.01 | 7.28 | 0.12 ± 0.01 | 7.20 |
| buffer, pH 7.5 | | | | |

TABLE 14
Intrinsic Dissolution Rate of Compound A1 Sesquifumarate Salt in
Simulated Biological Media (Mean ± SD, n = 2)

Compound A1 Sesquifumarate Salt

| Medium/ | | IDR |
|---|---|---|
| Initial pH | Final pH | (μg/min/cm²) |
| SGF, pH 1.3 | N/A | Too soluble to |
| | | be determined |
| FaSSIF, pH 6.51 | 6.42 | 553 ± 68.9 |
| FeSSIF, pH 5.03 | 4.93 | 33.5 ± 1.15 |

Thermal Behavior. The melting range of one sample of Form I of the sesquifumarate salt of Compound A1 was determined to be 154.3° C. with an onset at 141.7° C. Other thermal data is provided elsewhere herein.

Elemental Analysis. Combustion analysis of a sample of Form I of the sesquifumarate salt of Compound A1 was performed for the elements carbon, hydrogen, and nitrogen. Results are presented in Table 15. The actual values for C and N are lower than the theoretical values, but are in agreement with a drug substance containing absorbed water. The sesquifumarate salt has moisture absorption characteristics of a channel hydrate. Water vapor sorption experiments indicate that the drug substance equilibrates quickly to a moisture content of approximately 4.5% under typical laboratory humidity (55% RH). If a water content of 4.5% is factored into the calculations, the theoretical values are in agreement with the measured values (±0.4%).

TABLE 15
Elemental Analysis of Compound A1 Sesquifumarate

| | Theoretical (%) | Theoretical (%) | |
|---|---|---|---|
| Element | (moisture free) | (4.5% water Contest) | Actual (%) |
| C | 64.80 | 61.88 | 62.15 |
| H | 6.37 | 6.58 | 6.35 |
| N | 8.64 | 8.25 | 8.20 |

Mass Spectrometry. A mass spectrum of a sample of Form I of the sesquifumarate salt was obtained using electrospray ionization mass spectrometry (ESIMS) with positive ionization. The mass spectrum contains a major peak with an m/z value of 475.2859 corresponding to the protonated free base [MH]⁺. This value is in agreement with the theoretical exact mass (475.2873) within 3 ppm accuracy.

Nuclear Magnetic Resonance Spectroscopy. The identity of the sesquifumarate salt of Compound A1 was confirmed by ¹H and ¹³C NMR at 500 and 125 MHz respectively, in hexadeuterated dimethylsulfoxide with added tetramethylsilane (TMS) for reference. The ¹H spectrum was referenced to TMS (0.00 ppm) and the ¹³C spectrum was referenced to the NMR solvent (39.5 ppm). The ¹H and ¹³C assignments for the sesquifumarate salt of Compound A1 are shown in Table 16.

TABLE 16
¹H and ¹³C Chemical Shifts for the Sesquifumarate Salt of Compound A1

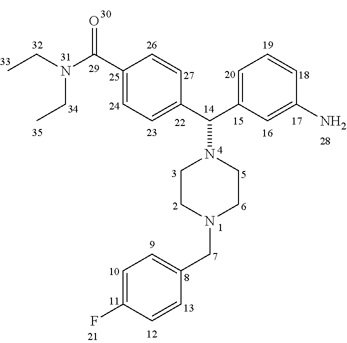

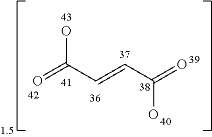

| | Chemical shift/ppm | |
|---|---|---|
| Atom | ¹H | ¹³C |
| 23, 27 | 7.42 (2H, d, J 8.1 Hz) | 127.4 |
| 9, 13 | 7.32 (2H, dd, J 8.4 Hz, J_{HF} 5.8 Hz) | 130.9 (d, J_{CF} 8.0 Hz) |
| 24, 26 | 7.25 (2H, d, J 8.1 Hz) | 126.2 |
| 10, 12 | 7.13 (2H, t, J 8.8 Hz, J_{HF} 8.8 Hz) | 114.9 (d, J_{CF} 21.1 Hz) |
| 19 | 6.91 (1H, t, J 7.7 Hz) | 128.9 |
| 36, 37 | 6.62 (2H, s) | 134.0 |
| 16 | 6.62 (1H, t, J 1.8 Hz) | 112.8 |
| 20 | 6.55 (1H, d, J 7.6 Hz) | 115.2 |
| 18 | 6.37 (1H, dd, J 1.3 Hz, J 7.9 Hz) | 112.7 |
| 14 | 4.11 (1H, s) | 75.0 |
| 7 | 3.54 (2H, s) | 60.6 |
| 32, 34 | 3.38 (2H, br), 3.16 (2H, br) | 42.8, 38.6 |
| 2, 6 | 2.47 (4H, br) | 52.3 |
| 3, 5 | 2.36 (2H, br), 2.30 (2H, br) | 51.0 |
| 33, 35 | 1.06 (6H, br) | 14.0, 12.8 |
| 29 | NR | 169.7 |
| 38, 41 | NR | 166.1 |
| 11 | NR | 161.3 (d, J_{CF} 242.7 Hz) |
| 17 | NR | 148.7 |
| 22 | NR | 143.9 |
| 15 | NR | 142.7 |
| 25 | NR | 135.6 |
| 8 | NR | 133.2 |

NR = No resonance.

The protons and the corresponding carbons for atoms 32, 33, 34 and 35 have broad peaks in the ¹H and ¹³C spectra due to rotamers around the —N—CO— bond. The ¹H spectrum recorded at 353K has a quartet for protons bound to atoms 32 and 34 and triplet for protons bound to atoms 33 and 35 as expected.

Infrared Spectroscopy. The infrared spectrum of a sample of Form I of the sesquifumarate salt of Compound A1 was recorded on the powder using attenuated total reflection (ATR) spectroscopy on a Fourier transform infrared spectrophotometer. Major absorption bands include: 2973 cm⁻¹ (NH stretch); 1725 cm⁻¹ (C═O fumaric acid stretch); 1708 cm⁻¹ (C═C fumaric acid stretch); 1613 cm⁻¹ (C═O amide stretch); 1563 cm⁻¹ (C═C aryl stretch).

Ultraviolet Spectroscopy. An ultraviolet spectrum of a 0.01 mg/mL solution of the sesquifumarate salt of Compound A1 in water was obtained scanning from 190 nm to 400 nm. The sesquifumarate salt has no discernible peak maxima and does not show significant light absorption at wavelengths greater than 290 nm.

Chemical Stability Data. The chemical stability of a sample of Form I of the sesquifumarate salt of Compound A1 was assessed by stressing the material at various temperature/relative humidity/light conditions, then analyzing by HPLC. Results are presented in Table 17 and Table 18.

TABLE 17
Chemical Stability Data for Form I of
the Sesquifumarate Salt of Compound A1

| | | | % Purity | % Purity | % Change |
|---|---|---|---|---|---|
| | Time point | | (reference | (after | from |
| Condition | (days) | | material) | challenge) | reference |
| Reference | 0 | | 100 | 99.6 | 0.0 |
| 25° C./ | 7 | | 104.0 | 99.6 | 0.0 |
| 60% RH | | | | | |
| 40° C./ | 7 | | 103.5 | 99.5 | −0.1 |
| 75% RH | | | | | |
| 80° C. | 7 | | 99.8 | 97.1 | −2.5 |
| Ultraviolet Light, | ICH | (1 day) | 100.2 | 99.5 | −0.1 |
| 25° C./ | | | | | |
| 60% RH* | | | | | |
| Ultraviolet light, | 2x ICH | (2 days) | 100.7 | 99.6 | 0.0 |
| 25° C./ | | | | | |
| 60% RH* | | | | | |
| Ultraviolet light, | 3x ICH | (3 days) | 100.9 | 99.4 | −0.2 |
| 25° C./ | | | | | |
| 60% RH* | | | | | |
| White light, | ICH | (7 days) | 101.7 | 99.5 | −0.1 |
| 25° C./60%* | | | | | |

*Light samples were stored open. Unlike other samples which were weighed prior to storage, the light samples were weighed for assay after storage.

TABLE 18
Chemical Stability Data for Form I of the Sesquifumarate Salt of Compound A1

| | | | | | Purity after |
|---|---|---|---|---|---|
| | Purity after | Purity after | Purity after | Purity after | Photostability |
| Initial | 60° C./80% RH | 60° C./80% RH | 80° C., | 80° C., | 3X ICH |
| Purity* | 2 weeks | 4 weeks | 2 weeks | 4 weeks | conditions |
| 98.6% | 86.5% | 80.3% | 91.8% | 86.1% | 93.2% |

*All purity values assessed by HPLC area %.

Impurity Analysis. Studies to characterize organic impurities (e.g., starting materials, by-products, intermediates, and degradation products) were performed on Form I of the sesquifumarate salt of Compound A1.

Organic impurities. The synthetic impurities and degradation products in a sample of the sesquifumarate salt were detected by reverse-phase HPLC with UV detection. Structures of 3 of the impurities, a fumarate adduct impurity, a formamide impurity, and a des-piperazine impurity, were assigned by LC/MS. Chemical structures for each of these impurities are provided herein.

The fumarate adduct impurity (relative retention time (RRT 1.50)) was the result of a Michael-type addition reaction between the Compound A1 primary amine functionality and fumaric acid and was detected at 0.17% and <0.05% in different batches. It was also a degradation product when the drug substance is stored at elevated temperatures and humidity.

The formamide impurity (RRT 1.52) was proposed to form through a formylation of the Compound A1 primary amine functionality and was detected at levels ≦0.1% in certain batches. It appeared to primarily form when the drug substance is stored at elevated temperature or under light stress.

The Compound A1 des-piperazine impurity (RRT 0.90) was a degradation product formed through oxidative loss of the fluorobenzyl piperazine moiety. It was formed in drug substance and drug product upon storage, with the rate of formation being accelerated under stress conditions (temperature, humidity and light).

There were 2 unidentified impurities present in both drug substance batches which do not increase upon storage, including stressed conditions. One impurity (RRT 1.71) was detected at 0.25% and 0.21% in certain batches. The second impurity (RRT 2.40) was detected at 0.15% and 0.24% in certain batches. No other impurities were observed at levels >0.10%.

Chiral impurities. Certain batches of the sesquifumarate salt were manufactured using an achiral synthesis followed by a chromatographic resolution step to isolate the desired R-enantiomer. The level of the undesired S-enantiomer was measured by capillary electrophoresis. The S-enantiomer was present at 1.5% in both batches. There is no evidence of isomerization of the S-enantiomer upon storage.

Inorganic impurities. In certain batches, platinum on carbon was used as a catalyst in the synthesis of the sesquifumarate salt. Platinum level in the drug substance were measured by ICP. Levels of platinum were <10 ppm.

Residual solvents analysis. In certain batches, no Class 1 solvents were used in the manufacture of the sesquifumarate salt. Certain batches of the sesquifumarate salt were manufactured using an achiral synthesis with the final steps in the process being a chromatographic resolution step in ethanol/2-propanol; followed by formation and isolation of the sesquifumarate salt from ethanol. Ethanol and 2-propanol are Class 3 solvents and are the only solvents which were present in the final product. Their levels in the final product were determined by gas chromatography. Solvents used earlier on in the synthesis were excluded from the final product during the chromatographic resolution step.

Solubility. The solubility of Form I of the sesquifumarate salt of Compound A1 was measured in various media. In water, the solubility was 24 mgA/ml (pH=3.8). In 0.1M phosphate acid, the solubility was 12 mgA/ml (pH=3.5). In 0.1N HCl, the solubility was 24.9 mgA/ml (pH about 2).

### Example 6

### In Vivo Biological Evaluations

### Anxiolytic Effect of Compound A1 in the Punished Responding Procedure (a Modified Geller-Seifter) in the Rat In Vivo

Objective. The purpose of the present study was to determine the anxiolytic efficacy of 4-{(R)-(3-aminophenyl)[4-(4-fluorobenzyl)piperazin-1-yl]methyl}-N,N-diethylbenzamide (hereinafter “Compound A1”) in the modified Geller-Seifter conflict test.

Method. In the conflict test, hungry animals were trained to lever-press for food delivery in a standard operant chamber under 2 conditions. In the first condition, referred to as the unsuppressed component, food was delivered on average after 17 lever-presses were made (also called a VR17 schedule of reinforcement). In the second condition, referred to as the suppressed component and signaled by flashing lights inside the operant chamber, food was also delivered following an average of 17 lever-presses, but electric shock was additionally delivered to the floor of the cage under a separate VR17 schedule. Daily sessions consisted of 5 alternating presentations of each component type: suppressed (3 minutes in duration) and unsuppressed (2 minutes in duration). The number of lever presses emitted in the suppressed component was obviously low relative to the unsuppressed component. The rats were dosed with Compound A (0.21 to 21 μmol/kg) and the rate of responding in unpunished and punished components recorded.

Drug Administration and Preparation. Compound A1 was dissolved in distilled deionized water/lactic acid and was administered by mouth in a volume of 1 mL/kg body weight. Compound A1 had a 60 minute pretreatment time. Compound A1 was administered on Tuesday and Friday and vehicle on Thursday. Monday and Wednesday were washout/baseline days.

Apparatus. Standard 2-lever operant chambers were used (Med Associates). The chambers were fitted with 2 retractable response levers and a stimulus lamp over each of the 2 levers. A pellet dispenser delivered 45 mg food pellets, (Bio Serv) to a cup located inside of the chamber below and between the 2 response levers. A lamp at the top and back of the chamber served as houselights. The grid floors of the operant chambers were interfaced to shock generators and scramblers (Med Associates). All events in the chambers were controlled and monitored by a microprocessor.

Procedures. There were 2 components in the procedure: 1) an unsuppressed responding component (unpunished) with 2 minutes in duration; and 2) a suppressed responding component (punished) with 3 minutes in duration.

In the unpunished component, the houselights and both stimulus lamps over the response levers were turned on, the lever on the left-hand side of the chamber extended, and a food pellet was delivered following an average of 17 responses on the lever in the chamber (range 3 to 40 responses)—a variable-ratio 17 schedule (VR17).

In the punished component (which followed the unpunished component), the right-hand lever was extended into the chamber; the stimulus lamps and houselights were turned on and off at 1 second intervals, in succession, which served as a cue for this component; and food was available under a VR17 schedule, but was accompanied by an electrical current (0.5 second duration) that was delivered to the grid floor of the chamber under an independent VR17 schedule. The level of the current was adjusted for each individual subject until responding was reduced in the punished component to a level that was about 5% to 10% that of the unpunished component, and ranged from 0.2 mA to 0.75 mA. Unpunished and punished components were separated by 10 second time-out periods in which both response levers were retracted and all stimulus lamps turned off. 2 minute unpunished and 3 minute punished components alternated until 5 of each were completed. Daily sessions always began with the unpunished responding component. For any given drug test, rats whose responding was most stable were chosen from a larger pool of trained rats. Several doses were tested on a given test day in different subjects. Each dose, then, was tested in different sub-set of rats.

Data Analysis. The dependent variables recorded were the rate of responding in unpunished and punished components (total responses/total time under the component) and the number of shocks delivered. A selective anxiolytic effect is defined as an increase in responding in the punished components with relatively less or no effect on responding in unpunished components. t-Tests were used to compare mean of the control's rate of responding on vehicle day of the rats used for a specific dose to the same rats means following delivery of each dose of Compound A1 (for only the rats used within each dose).

Results. The results of the present study are summarized in Table 19 and Table 20. Compound A1 increased the rate of punished responding at 0.63, 2.11, 6.3, and 21.1 μmol/kg compared to their vehicle controls. Compound A1 was maximally efficacious at 2.11 μmol/kg, producing about 300% increase in punished responding. Compound A1 at 0.21 μmol/kg was not effective in this model. Compound A1 did not significantly decrease unpunished responding at the doses tested.

TABLE 19
Rate of Responding Test Result Summary

| | Punished | | | Unpunished | | |
|---|---|---|---|---|---|---|
| Treatment | responding | Vehicle | P value | responding | Vehicle | P value |
| Vehicle | 0.10 ± 0.01 | ND | ND | 1.92 ± 0.05 | ND | ND |
| (average) | | | | | | |
| Compound A1 | 0.11 ± 0.02 | 0.11 ± 0.01 | 0.493 | 2.06 ± 0.11 | 2.01 ± 0.11 | 0.080 |
| 0.21 μmol/kg | | | | | | |
| Compound A1 | 0.24 ± 0.03 | 0.09 ± 0.01 | 0.003^{a} | 2.02 ± 0.14 | 2.19 ± 0.06 | 0.092 |
| 0.63 μmol/kg | | | | | | |
| Compound A1 | 0.18 ± 0.04 | 0.08 ± 0.02 | 0.046^{a} | 1.95 ± 0.15 | 1.87 ± 0.17 | 0.241 |
| 2.1 μmol/kg | | | | | | |
| Compound A1 | 0.17 ± 0.03 | 0.09 ± 0.01 | 0.031^{a} | 1.92 ± 0.13 | 1.81 ± 0.11 | 0.031^{a} |
| 6.3 μmol/kg | | | | | | |
| Compound A1 | 0.20 ± 0.04 | 0.10 ± 0.021 | 0.045^{a} | 1.69 ± 0.12 | 1.77 ± 0.09 | 0.169 |
| 21.1 μmol/kg | | | | | | |

^{a}= P value is associated with t-Test vs. individual vehicle group (p < 0.05)
ND = Not determined

TABLE 20
Percent of Control Test Result Summary

| | Punished | | | Unpunished | | |
|---|---|---|---|---|---|---|
| Treatment | responding | Vehicle | P value | responding | Vehicle | P value |
| Vehicle. | 95.5 ± 6.61 | ND | ND | 100 ± 2.85 | ND | ND |
| (average) | | | | | | |
| Compound A1 | 107 ± 19.3 | 110 ± 14.9 | 0.458 | 103 ± 1.63 | 105 ± 5.76 | 0.396 |
| 0.21 μmol/kg | | | | | | |
| Compound A1 | 273 ± 39.2 | 92.7 ± 8.70 | 0.005^{a} | 92.1 ± 5.12 | 114 ± 3.30 | 0.003^{a} |
| 0.63 μmol/kg | | | | | | |
| Compound A1 | 298 ± 132 | 80.4 ± 16.0 | 0.092 | 106 ± 5.60 | 97.3 ± 9.10 | 0.241 |
| 2.1 μmol/kg | | | | | | |
| Compound A1 | 207 ± 67.2 | 91.8 ± 11.7 | 0.079 | 106 ± 2.94 | 94.3 ± 5.80 | 0.031^{a} |
| 6.3 μmol/kg | | | | | | |
| Compound A1 | 272 ± 100 | 104 ± 24.0 | 0.107 | 95.5 ± 4.08 | 92.3 ± 4.64 | 0.317 |
| 21.1 μmol/kg | | | | | | |

^{a}= P value is associated with t-Test vs. individual vehicle group (p < 0.05)
ND = Not determined

Compound A1 has acute anxiolytic activity in a modified Geller-Seifter conflict model with a level of efficacy to that of diazepam (about 250% at 3.5 μmol/kg by mouth). Observations of test subjects in their home cage suggest Compound A1 does not possess sedative/hyperactive properties.

Antidepressant Effect of Compound A1 in the Learned Helplessness Procedure in the Rat

Objective. The purpose of the present study was to determine the antidepressant potential of 4-{(R)-(3-aminophenyl)[4-(4-fluorobenzyl)piperazin-1-yl]methyl}-N,N-diethylbenzamide (hereinafter “Compound A1”) in the learned helplessness test.

Method. Male Sprague Dawley rats were exposed to inescapable electrical stimulation for 2 successive, daily 1 hour periods (conditioning), and then subsequently trained to avoid or escape the electrical stimulation by running to the opposite side of the test cage. During conditioning and training, animals were injected twice per day with vehicle, imipramine HCl (15 mg/kg) or 0.1, 1 and 10 mg/kg Compound A1 over 2 separate studies. In the training phase, when the electrical stimulation was escapable, the number of escape failures was recorded. Standard shuttle cages (20 L×16 W×21 centimeters H) fitted with a grid floor were used. The chambers could be partitioned with a closed partition or with an archway that allowed the animals to pass between the 2 sides of the cage. A computer controlled and monitored all events in the chamber.

Procedure. The procedure utilized involved an induction phase and an avoidance training phase. In the induction phase, rats were enclosed in one side of the shuttle cage and electrical stimulation (2 mA, 9.9 second duration) was delivered to the floor of the cage every 2, 5 or 10 seconds (randomly selected for each trial) until 90 electrical stimulations were delivered. Subjects had no opportunity to escape or to avoid the electrical stimulation. Induction was conducted for 2 consecutive days. In the avoidance training phase, a partition with an arch through which the rats could pass was inserted into the center of the shuttle cages. The method employed a standard shuttle avoidance in which a compound, conditioned stimulus (a 5 second presentation of a tone and turning on of a lamp on the side of the rat containing cage) served to indicate impending presentation of electrical stimulation to the floor of the cage. The electrical stimulation was presented for a 5 second period 5 seconds after initiation of the conditioned stimulus. Entry into the opposite side of the shuttle cage via the arched partition prior to onset of electrical stimulation resulted in the end of the trial (avoidance response). If electrical stimulation was delivered, entry into the opposite side of the cage resulted in termination of the electrical stimulation and the Conditioned Stimulus (escape). A 30 second inter-trial interval was employed. 40 minute avoidance training sessions, consisting of 50 trials were conducted on 2 consecutive days, beginning 48 hours after the final induction session.

Drug Administration and Preparation. Dosages of all compounds are reported as the free base. Imipramine and Compound A1 were dissolved in distilled deionized water and administered by mouth in a volume of 1 mL/kg body weight. Drug was administered immediately following conditioning and training sessions and approximately 7-8 hrs after the first injection, as well as on the day in the middle of the study when no conditioning or training was conducted. In study 2, imipramine was administered 30 minutes prior to avoidance training whereas in all other circumstances, drug was administered following training

Data analysis. The primary dependent variable was escape failures during avoidance training. Additionally, because some delta opioid agonists have been shown to produce locomotor stimulation, center crossings during avoidance training were also recorded and compared among groups, which allows a gauge of motor activity. An increase in center crossings with respect to vehicle control suggests that locomotor stimulation may be partly or fully responsible for the putative antidepressant effects of the compound. T-Tests were used to compare the performance of the vehicle-administered group to drug treated groups. The no-induction group was used to gauge whether learned helplessness was established, by comparison to the vehicle treated group.

Results. The results of the present study are summarized in Table 21. Saline-treated rats exposed to inescapable electric stimulation (induction phase) failed to escape in the avoidance phase in 16 (on average) of the 50 trials. The study integrity was confirmed by a significantly reduced number of escape failures in saline-treated rats that were not exposed to inescapable electric stimulation and the imipramine-treated rats. All 3 doses of Compound A1 tended to decrease escape failures, although only the 2 higher doses were significantly different from the saline-treated rats exposed to inescapable electric stimulation. The reduction in escape failures was not due to increased locomotion per se, since center crossings in the cage in the treated groups was either not different or even slightly reduced compared to the saline-treated rats exposed to inescapable electric stimulation.

TABLE 21
Antidepressant Effect Results Summary

| | Mean Escape | | | Mean Center | | |
|---|---|---|---|---|---|---|
| Treatments | Failures ± (SEM) | | P Value | Crossings ± SEM | | P Value |
| IES + | 16.2 | (3.9) | — | — | | — |
| Saline | | | | | | |
| No IES + | 5.4 | (1.4) | <0.003* | 32.9 | (2.1) | >.05 |
| Saline | | | | | | |
| Imipramine | 2.3 | (1) | <0.002* | 30.2 | (1.1) | <0.02* |
| 20 mg/kg | | | | | | |
| Compound A1 | 6.1 | (3.4) | =0.06 | 26.6 | (0.85) | >0.05 |
| 0.1 mg/kg | | | | | | |
| Compound A1 | 3.1 | (1.7) | <0.004* | 30.9 | (2.9) | >0.05 |
| 1 mg/kg | | | | | | |
| Compound A1 | 4.9 | (2) | <0.01* | 26.9 | (0.7) | <0.02* |
| 10 mg/kg | | | | | | |

IES = inescapable electrical stimulation.
P value is associated with T test vs. IES + vehicle group.
All treatments were given by mouth twice a day.

Compound A1 produced a decrease in escape failures in the learned helplessness test, which is indicative of potential antidepressant action.

### Example 7

### Clinical Evaluations

Treatment of AMDD Patients with Form I of the Sesquifumarate Salt of Compound A1

A clinical study is performed to assess the safety and efficacy of a solid form comprising Compound A1or a salt thereof, as described herein, in the treatment of AMDD.

Overview. A randomized, double blind, placebo-controlled, parallel-group experimental study was designed to assess the efficacy and safety of 3 mg of Form I of the sesquifumarate salt of 4-{(R)-(3-aminophenyl)[4-(4-fluorobenzyl)piperazin-1-yl]methyl}-N,N-diethylbenzamide (hereinafter “Compound A1 sesquifumarate”) given twice daily over 4 weeks to male and female patients ages 18 to 65 years meeting diagnostic criteria for AMDD but without psychotic features, according to a Hamilton Rating Scale for Depression (hereinafter “HRSD17”) and Hamilton Psychiatric Rating Scale for Anxiety (hereinafter “HAM-A”) total scores and DSM-IV and confirmed by the Structured Clinical Interview for the Diagnostic Manual of Mental Disorders, Fourth Edition, Patient Version (hereinafter “SCID-I/P”) (See, First M B, Spitzer R L, Gibbon M, Williams A R (2001): Structured Clinical Interview for DSM-IV TR Axis I Disorders, Research Version, Patient Edition. New York: New York State Psychiatric Institute, Biometrics Research).

More specifically, patients involved in this study will have (1) documented clinical diagnosis meeting criteria from the DSM-IV for at least one of the following: 296.22 Major Depressive Disorder, Single Episode, Moderate; 296.23 Major Depressive Disorder, Single Episode, Severe Without Psychotic Features, duration at least 1 year; 296.32, Major Depressive Disorder, Recurrent, Moderate; and/or 296.33, Major Depressive Disorder, Recurrent, Severe Without Psychotic Features; and (2) a HRSD17 total score 20; a HAM-A total score 16; and a Clinical Global Impressions Severity (hereinafter “CGI-S”) score 4 at both enrollment and randomization.

The HRSD17 is a widely used observational rating measure of depression severity. (See, Hamilton M (1960b): A rating scale for depression. J. Neurol. Neurosurg. Psychiatry 23:56-62). The 17-item version of this scale, which also referred to as HAMD, will be administered to assess the severity of depression. The HAMD assesses both the presence and severity of individual signs and symptoms characterizing depression without psychotic features.

HAM-A is a widely used observational rating measure of anxiety severity. The scale consists of 14 items. Each item is rated on a scale of 0 to 4. This scale will be administered to assess the severity of anxiety and its improvement during the course of therapy. The HAM-A total score is the sum of the 14 items and the score ranges from 0 to 56.

The Hamilton Rating Scale for Depression Anxiety/Somatization Subscale (hereinafter “HRSD17(A/S)”) is a subscale of the HRSD17. It factors scores derived from items 10, 11, 12, 13, 15, and 17 of the HRSD17.

The SCID I/P is a semi-structured interview. It is administered by a clinician to diagnose psychiatric illness and provides probe questions as well as follow-up questions to assist the clinician in diagnosis. It includes an overview to obtain information about demographics, work, chief complaint, history of present illness, past history, treatment history, and current functioning. The main body of the SCID I/P includes 9 modules designed to diagnose 51 mental illnesses in all. The modules of the research version can be tailored to needs, purpose, and goals of the investigation. It includes sections on current as well as past psychiatric disorders.

CGI-I/S scale is a three-item scale that assesses treatment response in psychiatric patients. (See, Guy W (1976): ECDEU Assessment Manual for Psychopharmacology, Revised). The administration time is 5 minutes. The scale consists of three items: Severity of Illness (item 1); Global Improvement (item 2); and Efficacy Index (item 3). Item 1 is rated on a seven-point scale from 1, which is normal to 7, which is among the most extremely ill patients. Item 2 is also rated on a seven-point scale from 1, which is very much improved to 7, which is very much worse. Each includes an additional response of “not assessed”. Item 3 is rated on a four-point scale from “none” to “outweighs therapeutic effect”. Items 1 and 3 are assessed based on the previous week's experience. Item 2 is assessed from the period since the initiation of the current treatment.

CGI-S scale is the Item 1 Severity of Illness scale of the CGI-I/S scale.

Approximately 96 subjects with AMDD will be screened to randomize 80 in this study. Randomization will be performed at a 2:1 ratio into the following two groups:

Treatment Group A: On the morning of Day 1, all patients in treatment group A will receive a 3 mg dose of Compound A1 sesquifumarate. On Day 2, the dose will be increased 3 mg of Compound A1 sesquifumarate twice a day. Patients will continue on 3 mg of Compound A1 sesquifumarate twice a day for approximately 28 days.

Treatment Group B: Patients in treatment Group B will receive placebo capsules matching the color, size and appearance of the capsules received by treatment group A. The regimen for dosing will be identical to Treatment Group A's dosing regimen.

Dose selection: In rodents, doses of Compound A1 sesquifumarate that produced mean plasma exposures ≧2 ng/ml have been shown to be efficacious in separate tests of anxiolysis and antidepressant activity. Monte Carlo simulations (N=1000) based on human pharmacokinetic data for Compound A1 collected in the Phase I program (N=96 male subjects) estimates 3 mg/kg twice a day will achieve an average plasma exposure of ≧2 ng/ml in 96% of subjects upon initial dosing, increasing to >98% of subjects at steady state.

Screening Period. The screening period will be up to 30 days prior to Day 1 of the Treatment Period. All patients will be required to stop current antidepressant treatment at least 14 days prior to Day 1 of the Treatment Period. Patients may be admitted to the Clinical Research Center (hereinafter “CRC”) during the washout period based on deterioration of their depressive symptoms.

Treatment Period. Day 1 to Day 7: On Day 1 of the Treatment Period, patients will be randomized to receive either Compound A1 sesquifumarate (Treatment Group A) or placebo (Treatment Group B) based on the randomization schedule.

Day 7 to Day 28: Patients will return to the CRC for 3 scheduled visits at Weeks 2, 3 and 4 during the Treatment Period for safety, tolerability, and efficacy assessments.

Follow-up Visit. Patients will be asked to return for a follow-up visit 7-10 days after completion of the outpatient period. During the follow-up visit, additional safety, tolerability, and efficacy assessments will be administered.

Data Analysis. This study is designed to assess the efficacy of 28 days of 3 mg of Compound A1 sesquifumarate by mouth twice a day compared with placebo in improving overall depressive symptomatology in patients with AMDD. The primary efficacy analysis will compare the response rates of Compound A1 sesquifumarate versus placebo groups in the intent-to-treat sample at study endpoint. Response will be defined as a reduction of 50% in the HRSD17 or HAM-A total score, and a CGI-I score=1 or 2 at Week 4 of treatment or study endpoint if the study is not completed. Additionally, the following analyses of HRSD17 and HAM-A data will be conducted:
Change from baseline to day of discharge from the CRC and change from baseline to Weeks 1, 2, 3, and 4 of treatment and change from baseline to follow-up in HRSD17 and HAM-A total scores.
Change from baseline to day of discharge from the CRC and change from baseline to Weeks 1, 2, 3, and 4 of treatment and change from baseline to follow-up in HRSD17 (A/S), and HRSD17, Item 10 (Anxiety Psychic) scores.
Time to anxiolytic response: defined as first assessment of a 50% reduction in HAM-A or HRSD17 (A/S) score using Kaplan-Meier survival analysis method.
Time to antidepressant response: defined as first assessment of a 50% reduction in HRSD17 total score using Kaplan-Meier survival analysis method.

All statistical comparisons will be based on a 2-tailed test using an alpha level of 0.05 unless otherwise specified. No correction to the reported p-values will be made for the primary analysis.

Descriptive statistics for continuous data will include number (n), mean, median, standard deviation, minimum and maximum value. Descriptive data for categorical data will include n, frequency, and percentage.

Efficacy analyses will be performed in the intent-to-treat population, and safety analyses will be performed in the safety population unless otherwise specified.

Descriptive statistics will be provided for all efficacy variables. Additionally, the ANCOVA model will be used for continuous variable and the logistic regression model will be used for categorical variables. For the time to event variables, Cox proportional hazards model will be used.

Clinical assessments including HRSD17, HAM-A, and CGI-I/S will be conducted. The HRSD17 will be used to collect information on depressive symptoms; the HAM-A will be used to collect data on anxiety symptoms; and the CGI-I/S will be used to collect data on overall severity of improvement/illness. In addition, information on depression, anxiety, pain symptoms, and suicidal ideation will be collected using the Inventory of Depressive Symptomatology, Clinician and Subject Rated (hereinafter “IDS-C₃₀/IDS-S₃₀”), the Depression Anxiety Stress Scale (hereinafter “DASS₄₂”), the 23-item Kellner Somatic Symptom Questionnaire (hereinafter “SSQ”), and a pain scale. The proportion who show a clinically significant change from baseline to endpoint in the HRSD17 or the HAM-A total scores (Hamilton 1960a; Hamilton 1967) as well as the CGI-I/S will serve as the primary efficacy measure for the study. Secondary efficacy assessments include the HRSD17, HAM-A, CGI-I/S, Inventory of Depressive Symptomatology, Clinician and Subject Rated (hereinafter “IDS-C₃₀/IDS-S₃₀”), Depression Anxiety Stress Scale (hereinafter “DASS₄₂”), and the pain assessment. In addition to indicating simple change in severity, the HRSD17 total score will be used to dichotomize patients into response versus nonresponse categories at the end of the Study. A responder will be defined as any patient who demonstrates a 50% or greater decrease in HRSD17 total score from baseline to endpoint. A remitter will be defined as any patient who demonstrates a <7 HRSD17 total score.

The IDS-C₃₀/IDS-S₃₀ is a 30-item observational rating measure of depression severity. The estimated time to administer this scale is 10 minutes. The scores on this measure can range from 0 to 84.

The DASS₄₂ is a set of 3 self-report scales measuring the negative emotional states of depression, anxiety and stress. This 42-item questionnaire has been shown to have high internal consistency and to yield meaningful discriminations in a variety of settings. The depression scale assesses dysphoria, hopelessness, devaluation of life, self-deprecation, lack of interest/involvement, anhedonia, and inertia. The anxiety scale assesses autonomic arousal, skeletal muscle effects, situational anxiety, and subjective experience of anxious affect. The stress scale is sensitive to levels of chronic non-specific arousal. It assesses difficulty relaxing, nervous arousal, and being easily upset/agitated, irritable/over-reactive and impatient.

The SSQ is a 23-item somatic scale composed of items that include both negative (17 items) and positive (6 items) somatic symptoms. The 17 negative somatic symptoms are as follows: feeling of not having enough air, heavy arms or legs, appetite poor, tight head and neck, choking feeling, feeling of pressure in head or body, weak arms or legs, breathing difficult, parts of the body feel numb or tingling, heart beating fast or pounding, pressure on head, nauseated/sick to stomach, upset bowels or stomach, muscles pains, headaches, cramps, and head pains. The six positive somatic symptoms are as follows: feeling healthy, feeling fit, no pains anywhere, arms and legs feel strong, no aches anywhere, and no unpleasant feelings in head or body.

Exemplary pain scales include but are not limited to, for example, the VAS and Likert Pain Scales. The VAS pain scale is a visual analog scale that assists patients in subjectively rating pain or sequelae of pain. The VAS is a straight line (100 mm) with the left end of the line representing no pain or symptoms and the right end of the line representing the worst pain or related symptoms imaginable. Patients rate their pain/symptoms by marking on the line where they feel their pain/symptoms lie. Likert scales are numbered scales to indicate level of pain.

What is claimed is:

1. A solid form comprising a salt of the compound of formula (I): 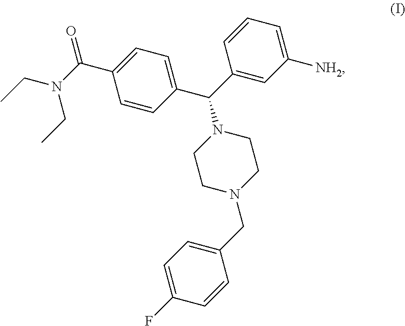
wherein the salt is selected from the group consisting of monohydrochloride, mesylate, and sesquifumarate.

2. The solid form of claim 1, which comprises the monohydrochloride salt.

3. The solid form of claim 2, which comprises between about 0.75 and about 1.25 molar equivalents of chloride ion per mole of the compound of formula (I).

4. The solid form of claim 2, which comprises about one molar equivalent of chloride ion per mole of the compound of formula (I).

5. The solid form of claim 2, which is substantially free of a hydrochloride salt of the compound of formula (I) with a stoichiometry other than about 1:1.

6. The solid form of claim 2, which is substantially amorphous.

7. The solid form of claim 6, which has an X-ray powder diffraction pattern which matches at least one X-ray powder diffraction pattern provided in FIG. 12.

8. The solid form of claim 6, which has an X-ray powder diffraction pattern which matches the X-ray powder diffraction pattern provided in FIG. 13.

9. The solid form of claim 6, which has an X-ray powder diffraction pattern comprising fewer than 10 peaks.

10. The solid form of claim 6, which has an X-ray powder diffraction pattern comprising a halo with a maximum between about 12 and 25 degrees 2θ.

11. The solid form of claim 6, which has an X-ray powder diffraction pattern matching the X-ray powder diffraction pattern provided

12. The solid form of claim 6, which has a glass transition temperature between about 30° C. and about 130° C.

13. The solid form of claim 6, which has a thermal gravimetric analysis thermogram comprising a mass loss of between about 0% and about 10% of the total mass of the sample.

14. The solid form of claim 6, which has a moisture sorption isotherm plot comprising a mass gain of less than about 20%.

15. The solid form of claim 6, which is substantially chemically pure.

16. The solid form of claim 6, which is substantially physically stable.

17. The solid form of claim 6, which contains birefringent particles comprising less than about 50% of the total number of particles in the sample.

18. The solid form of claim 6, which is obtainable from a solvent system comprising methanol.

19. The solid form of claim 6, which is obtainable from a procedure comprising spray drying.

20. The solid form of claim 6, which is obtainable from a procedure comprising vacuum drying.

21. The solid form of claim 2, which is substantially crystalline.

22. The solid form of claim 21, which further comprises solvent.

23. The solid form of claim 21, which comprises water.

24. The solid form of claim 21, which has an X-ray powder diffraction pattern comprising peaks at approximately 7.0, 11.0, and 16.8 degrees 2θ when analyzed using copper Kα radiation.

25. The solid form of claim 24, which has an X-ray powder diffraction pattern further comprising peaks at approximately 19.1, 19.8, and 20.3 degrees 2θ when analyzed using copper Kα radiation.

26. The solid form of claim 25, which has an X-ray powder diffraction pattern further comprising peaks at approximately 12.0, 17.4, and 19.3 degrees 2θ when analyzed using copper Kα radiation.

27. The solid form of claim 21, which has an X-ray powder diffraction pattern which matches the X-ray powder diffraction pattern provided in FIG. 2.

28. The solid form of claim 21, which has an X-ray powder diffraction pattern which matches the X-ray powder diffraction pattern provided in FIG. 3.

29. The solid form of claim 21, which has an X-ray powder diffraction pattern which matches at least one of the X-ray powder diffraction patterns provided in FIG. 4.

30. The solid form of claim 21, which has an X-ray powder diffraction pattern which matches at least one of the X-ray powder diffraction patterns provided in FIG. 5.

31. The solid form of claim 21, which has a differential scanning calorimetry thermogram comprising an endothermic event comprising an onset temperature between about 130° C. and about 160° C.

32. The solid form of claim 21, which has a thermal gravimetric analysis thermogram comprising a mass loss of between about 0% and about 15% of the total mass of the sample.

33. The solid form of claim 21, which has unit cell parameters consistent with the following approximate unit cell parameters when measured at a temperature of 173 K: a=18.43 Å; b=18.43 Å; c=18.67 Å; α=90°; β=90°; γ=90°; V=6344.2 Å³; and Z=8.

34. The solid form of claim 21, which has a moisture sorption isotherm plot comprising a mass gain of less than about 20% up to about 90% relative humidity.

35. The solid form of claim 1, which comprises the mesylate salt.

36. The solid form of claim 35, which is Form I of the mesylate salt of the compound of formula (I).

37. The solid form of claim 36, which is substantially crystalline.

38. The solid form of claim 37, which has an X-ray powder diffraction pattern comprising peaks at approximately 8.0, 17.5, and 22.9 degrees 2θ when analyzed using copper Kα radiation.

39. The solid form of claim 38, which has an X-ray powder diffraction pattern further comprising peaks at approximately 15.5, 19.5, and 20.5 degrees 2θ when analyzed using copper Kα radiation.

40. The solid form of claim 39, which has an X-ray powder diffraction pattern further comprising peaks at approximately 6.5, 10.7, and 21.6 degrees 2θ when analyzed using copper Kα radiation.

41. The solid form of claim 37, which has an X-ray powder diffraction pattern which matches the X-ray powder diffraction pattern provided in FIG. 28.

42. The solid form of claim 37, which has a moisture sorption isotherm plot comprising a mass gain of less than about 5% up to about 70% relative humidity

43. The solid form of claim 1, which comprises the sesquifumarate salt.

44. The solid form of claim 43, which comprises between about 1.25 and about 1.75 molar equivalents of fumarate ion per mole of the compound of formula (I).

45. The solid form of claim 43, which is Form I of the sesquifumarate salt of the compound of formula (I).

46. The solid form of claim 45, which is substantially crystalline.

47. The solid form of claim 45, which has an X-ray powder diffraction pattern comprising peaks at approximately 4.0, 8.0, and 24.1 degrees 2θ when analyzed using copper Kα radiation.

48. The solid form of claim 47, which has an X-ray powder diffraction pattern further comprising peaks at approximately 16.0, 17.9, and 19.9 degrees 2θ when analyzed using copper Kα radiation.

49. The solid form of claim 48, which has an X-ray powder diffraction pattern further comprising peaks at approximately 18.8, 19.2, and 25.4 degrees 2θ when analyzed using copper Kα radiation.

50. The solid form of claim 45, which has an X-ray powder diffraction pattern which matches the X-ray powder diffraction pattern provided in FIG. 38.

51. The solid form of claim 45, which has a differential scanning calorimetry thermogram comprising an onset temperature between about 100° C. and about 160° C.

52. The solid form of claim 45, which has a thermal gravimetric analysis thermogram comprising a weight loss of between about 0% and about 10% of the total mass of the sample.

53. The solid form of claim 45, which has a moisture sorption isotherm plot comprising a mass gain of less than about 10% up to about 90% relative humidity.

54. The solid form of any one of claims 2, 6, 21, 35, or 45, which is chemically pure.

55. The solid form of any one of claims 2, 6, 21, 35, or 45, which is physically pure.

56. A pharmaceutical composition comprising the solid form of any one of claims 2, 6, 21, 35, or 45.

57. The pharmaceutical composition of claim 56, which is a pharmaceutical composition for oral administration.

58. The pharmaceutical composition of claim 56, which is a solid pharmaceutical composition.

59. A method of treating a disease or disorder that can be treated by administration of a δ-opioid receptor ligand, which comprises administering the solid form of any one of claims 2, 6, 21, 35, or 45.

60. The method of claim 59, wherein the disease or disorder is selected from anxiety, depression, pain, and anxious major depressive disorder.

61. The solid form of any one of claims 2, 6, 21, 35, or 45, for use in therapy.

62. The solid form of any one of claims 2, 6, 21, 35, or 45, for use in treatment of a disease or disorder selected from anxiety, depression, pain, and anxious major depressive disorder.

63. The solid form of any one of claims 2, 6, 21, 35, or 45, for use in the manufacture of a medicament for treating a disease or disorder selected from anxiety, depression, pain, and anxious major depressive disorder.

64. An isolated Form I mono-HCl salt of Compound A1, which has an XRPD pattern comprising peaks at about 7.0, 11.0 and 16.8 degrees 2θ when analyzed using copper Kα radiation.

65. An isolated Form I mesylate salt of Compound A1, which has an XRPD pattern comprising peaks at about 8.0, 17.5, and 22.9 degrees 2θ when analyzed using copper Kα radiation.

66. An isolated Form I sesquifumarate salt of Compound A1, which has an XRPD pattern comprising peaks at about 4.0, 8.0, and 24.1 degrees 2θ when analyzed using copper Kα radiation.

67. The use of a solid form of any one of claims 2, 6, 21, 35, or 45 in the manufacture of a medicament for the therapy of a disease or disorder that can be treated by administration of a δ-opioid receptor ligand.

68. The method of claim 67, wherein the disease or disorder is selected from anxiety, depression, pain, and anxious major depressive disorder.
